SEQUENCE LISTING (59 sequences)

SEQ ID NO: 1 - DNA, 789 bp, Corynebacterium glutamicum
gtgactgatttttctagtgcttcaaatgctgacgattccacgcaggacggtcgtcctggtcgacgtgctggaaagtctaagaaggaatcgaagccaactccgtggtacatcgaaattccagtggttgtggttctgaccctcgcgctgattttcgtgctccagacgtttgtcggacgcatgtacatgattccgagtggttcgatggaacctactttgcacggatgtgagggctgcacgggtgaccgcatcctggtggagaaggtttcttactacttcacggatccagagccgggcgatgttgtggtgttcaagggtactgattcctggaacgttggattcactacgcagcgttccgataattcggtgatccgcggcctgcagaacctgggttcttacgtgggtcttgtcgcacctgatgaaaatgacctggtcaagcgcattatcgccaccggcggtcagactgtttcgtgccaagccggtgatcctggaatcatggttgacggcaaggaagtcgatgacagctacacgctgcaacctgcgcaattccccatcgatgagacctccggttccaccgaatgcggcggcaactatttcggccccatcaccgtgcctgacggcaactacttcatgatgggtgacaaccgcaccaactccatggattcccgctaccacctgggcgatcagtaccaaggaaccatccctgaggaaaacatcaagggcaaagttcaagcaattatcctgccatttagccgaatcggtggcgtcgacgaccctgccatcaaaggctag

SEQ ID NO: 2 - protein, 262 aa, Corynebacterium glutamicum
MTDFSSASNADDSTQDGRPGRRAGKSKKESKPTPWYIEIPVVVVLTLALIFVLQTFVGRMYMIPSGSMEPTLHGCEGCTGDRILVEKVSYYFTDPEPGDVVVFKGTDSWNVGFTTQRSDNSVIRGLQNLGSYVGLVAPDENDLVKRIIATGGQTVSCQAGDPGIMVDGKEVDDSYTLQPAQFPIDETSGSTECGGNYFGPITVPDGNYFMMGDNRTNSMDSRYHLGDQYQGTIPEENIKGKVQAIILPFSRIGGVDDPAIKG

SEQ ID NO: 3 - DNA, 30 bp, Artificial Sequence
  primer
gctaatcttcatggtaccaggcaaaaggta

SEQ ID NO: 4 - DNA, 35 bp, Artificial Sequence
  primer
gggggcgtcgatctagagggccctagcctttgatg

SEQ ID NO: 5 - DNA, 34 bp, Artificial Sequence
  primer
cgatattatttaggggtacccaaattcctgtgaa

SEQ ID NO: 6 - DNA, 34 bp, Artificial Sequence
  primer
gctaccttttgcctggtacctcacggccagccct

SEQ ID NO: 7 - DNA, 34 bp, Artificial Sequence
  primer
gccatcaaaggctagggccccaaattcctgtgaa

SEQ ID NO: 8 - DNA, 34 bp, Artificial Sequence
  primer
aggatccccggaatgggccctcacggccagccct

SEQ ID NO: 9 - DNA, 34 bp, Artificial Sequence
  primer
gctaccttttgcctggtaccttaaaatccacagc

SEQ ID NO: 10 - DNA, 34 bp, Artificial Sequence
  primer
aggatccccggaatgggcccttaaaatccacagc

SEQ ID NO: 11 - protein, 263 aa, Unknown [sequence omitted]

SEQ ID NO: 12 - DNA, 792 bp, Unknown
  MULTISPECIES
cacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 13 - DNA, 34 bp, Artificial Sequence
  primer
gctaccttttgcctggtaccttaccaatgcttaa

SEQ ID NO: 14 - DNA, 34 bp, Artificial Sequence
  primer
aggatccccggaatgggcccttaccaatgcttaa

SEQ ID NO: 15 - protein, 109 aa, Escherichia coli
ASDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 16 - DNA, 330 bp, Escherichia coli
gcgtccgataagattattcaccttaccgatgactccttcgataccgatgtcctcaaggcagatggcgctattctcgtggacttctgggcagaatggtgcggcccatgcaagatgatcgccccaattctcgatgaaatcgcggacgagtaccagggcaagttgaccgtggcaaaactgaacattgatcagaacccaggcaccgctcctaagtacggcatccgcggtattcctaccctgctccttttcaaaaacggtgaggtcgcagctaccaaagtgggtgcgttgtctaagggtcagctcaaagagttcctggatgctaacctggcgtga

SEQ ID NO: 17 - DNA, 34 bp, Artificial Sequence
  primer
gctaccttttgcctggtacctcacgccaggttag

SEQ ID NO: 18 - DNA, 34 bp, Artificial Sequence
  primer
aggatccccggaatgggccctcacgccaggttag

SEQ ID NO: 19 - protein, 127 aa, Homo sapiens
MSFSGKYQLQSQENFEAFMKAIGLPEELIQKGKDIKGVSEIVQNGKHFKFTITAGSKVIQNEFTVGEECELETMTGEKVKTVVQLEGDNKLVTTFKNIKSVTELNGDIITNTMTLGDIVFKRISKRI

SEQ ID NO: 20 - DNA, 384 bp, Homo sapiens
atgtccttctccggcaagtaccagctgcagtcccaggaaaacttcgaggcattcatgaaggctatcggtctgccagaagagctcatccagaagggcaaggatatcaagggtgtttccgaaatcgtgcagaacggcaagcacttcaagttcaccatcaccgcaggttccaaggtcatccagaacgagttcaccgttggcgaagagtgcgaactcgagaccatgaccggtgaaaaggttaagaccgtggtccagctggagggcgacaacaagctcgtgaccaccttcaagaacatcaagtccgtcaccgaactgaacggcgatatcatcaccaacaccatgaccctcggtgacatcgtgttcaagcgcatctccaagcgtatctaa

SEQ ID NO: 21 - DNA, 34 bp, Artificial Sequence
  primer
gctaccttttgcctggtaccttagatacgcttgg

SEQ ID NO: 22 - DNA, 34 bp, Artificial Sequence
  primer
aggatccccggaatgggcccttagatacgcttgg

SEQ ID NO: 23 - DNA, 34 bp, Artificial Sequence
  primer
acgaattcgagctcggtacccaaattcctgtgaa

SEQ ID NO: 24 - DNA, 34 bp, Artificial Sequence
  primer
ctagaggatccccgggtacctcacggccagccct

SEQ ID NO: 25 - DNA, 34 bp, Artificial Sequence; identical to SEQ ID NO: 6
  primer

SEQ ID NO: 26 - DNA, 34 bp, Artificial Sequence; identical to SEQ ID NO: 7
  primer

SEQ ID NO: 27 - DNA, 34 bp, Artificial Sequence
  primer
aggtcgactctagagggccctcacggccagccct

SEQ ID NO: 28 - DNA, 1458 bp, Corynebacterium glutamicum
atggaaaacccttatgtcgctgcgctcgatgacgataaaaaagaagtcggcgcaataaaagaagcagaaaaagaacctgaaataggtcccatcagagctgccggacgagccataccgctgcgcacccgcatcattttgatcgtggtgggtatcgccgggcttggtttgctggtcaacgcgattgctgtttccagcctcatgcgtgaagtttcctatacccgcatggatcaagagctagagacctcgatggggacgtgggcgcataacgttgagctgtttaatttcgatggcgtccgccaagggccacccagcgattattatgtggccaaggtttttcctgatggatccagcattattttcaacgatgcacaatcggcacccaatctagctgaaaccaccatcggtactggtccacacactgtggatgctgctagcggttctgcctccaacactccgtggcgtgtgatggcggaaaagaacggtgacattatcaccgtggtgggtaaaagcatggggcgtgaaacaaacctgctgtaccgattggtgatggtgcagatgatcatcggcgcgctgattctggttgctattttgattacttcactcttcctagtcagacgctcgttgcggccgttgagagaagttgaagagaccgccaccaggattgcgggcggtgatttggatcgacgtgtcccgcagtggccaatgaccacagaagtcggacagctgtcgaatgccctcaatatcatgttggagcagctccaagcctcaattctgaccgcccagcaaaaagaagctcagatgcgccgattcgttggcgacgcctcccacgagctccgcacaccactgacctctgtgaagggcttcaccgagctgtattcatcaggtgcaacagatgatgccaactgggtcatgtccaagatcggtggcgaagcccaacgcatgagtgtgcttgtggaagacctcctgtcactgacgcgtgccgaaggccagcaaatggagaagcaccgcgttgacgtgctggaactcgcattggcagtacgcggatccatgcgagcagcctggccagatcgcaccgtcaacgtgtccaataaagccgagtccattccagttgttgaaggcgacccaacccgcctccaccaagttctcaccaacctggttgccaacggactcaaccacggcggaccggacgcggaagtcagcattgagatcaacaccgatgggcaaaacgtgaggattctcgtggcagacaacggtgtcggaatgtctgaagaagatgcccagcatatcttcgagcgtttctaccgcgccgattcctcccgctcacgcgcatccggcggatcgggcctcggccttgcgatcacgaaatccctggtcgaaggccacggcggcacagtcaccgtcgacagcgtgcaaggcgaaggcacggtgttcacgatcaccttgccggcggtttcttaa

SEQ ID NO: 29 - protein, 485 aa, Corynebacterium glutamicum
MENPYVAALDDDKKEVGAIKEAEKEPEIGPIRAAGRAIPLRTRIILIVVGIAGLGLLVNAIAVSSLMREVSYTRMDQELETSMGTWAHNVELFNFDGVRQGPPSDYYVAKVFPDGSSIIFNDAQSAPNLAETTIGTGPHTVDAASGSASNTPWRVMAEKNGDIITVVGKSMGRETNLLYRLVMVQMIIGALILVAILITSLFLVRRSLRPLREVEETATRIAGGDLDRRVPQWPMTTEVGQLSNALNIMLEQLQASILTAQQKEAQMRRFVGDASHELRTPLTSVKGFTELYSSGATDDANWVMSKIGGEAQRMSVLVEDLLSLTRAEGQQMEKHRVDVLELALAVRGSMRAAWPDRTVNVSNKAESIPVVEGDPTRLHQVLTNLVANGLNHGGPDAEVSIEINTDGQNVRILVADNGVGMSEEDAQHIFERFYRADSSRSRASGGSGLGLAITKSLVEGHGGTVTVDSVQGEGTVFTITLPAVS

SEQ ID NO: 30 - protein, 485 aa, Corynebacterium glutamicum
MENPYVAALDDENQEVGVKKEAEKEPEIGPIRAAGRAIPLRTRIILIVVGIAGLGLLVNAIAVSSLMREVSYTRMDQELETSMGTWAHNVELFNFDGVRQGPPSDYYVAKVFPDGSSIIFNDAQSAPDLAETTIGTGPHTVDAASGSASNTPWRVMAEKNGDIITVVGKSMGRETNLLYRLVMVQMIIGALILVAILITSLFLVRRSLRPLREVEETATRIAGGDLDRRVPQWPMTTEVGQLSNALNIMLEQLQASILTAQQKEAQMRRFVGDASHELRTPLTSVKGFTELYSSGATDDANWVMSKIGGEAQRMSVLVEDLLSLTRAEGQQMEKHRVDVLELALAVRGSMRAAWPDRTVNVSNKAESIPVVKGDPTRLHQVLTNLVANGLNHGGPDAEVSIEINTDGQNVRILVADNGVGMSEEDAQHIFERFYRADSSRSRASGGSGLGLAITKSLVEGHGGTVTVDSVQGEGTVFTITLPAVS

SEQ ID NO: 31 - protein, 485 aa, Corynebacterium glutamicum
MENPYVAALDDENQEVGVKKEAEKEPEIGPIRAAGRAIPLRTRIILIVVGIAGLGLLVNAIAVSSLMREVSYTRMDQELETSMGTWAHNVELFNFDGVRQGPPSDYYVAKVFPDGSSIIFNDAQSAPDLAETTIGTGPHTVDAASGSASNTPWRVMAEKNGDIITVVGKSMGRETNLLYRLVVVQMIIGALILVAILITSLFLVRRSLRPLREVEETATRIAGGDLDRRVPQWPMTTEVGQLSNALNIMLEQLQASILTAQQKEAQMRRFVGDASHELRTPLTSVKGFTELYSSGATDDANWVMSKIGGEAQRMSVLVEDLLSLTRAEGQQMEKHRVDVLELALAVRGSMRAAWPDRTVNVSNKAESIPVVEGDPTRLHQVLTNLVANGLNHGGPDAEVSIEINTDGQNVRILVADNGVGMSEEDAQHIFERFYRADSSRSRASGGSGLGLAITKSLVEGHGGTVTVDSVQGEGTVFTITLPAVS

SEQ ID NO: 32 - protein, 504 aa, Corynebacterium callunae
MENPYVAALDKNSNFGAKDTDSAVSDSTEVSQNNDGIGTPATAEPKVGPIRTAGRAMPLRTRIILLVVGIAGLGLLVNAVAVSSLMREVSYTRMDQDLESAMGTWVRNVELFNFDGVRQGPPSDYYVAKVFPDGSSIIFNDAESAPDLGQTTIGTGPHTVEAAEGSASSTHWRVMAAKNGDVITVVGKSMGRESTLLYRLVVVQMVIGVLILIAILIGSFFLVRRSLKPLREVEETASRIAGGELDRRVPQWPMTTEVGQLANALNIMLEQLQTSIMNAQQKEAQMRRFVGDASHELRTPLTSVKGFTELYSSGATQDADWVLSKIGGEAQRMSVLVEDLLSLTRAEGQQMEKHRVDMLELALAVRGSLKAAWPDRTVNVANRSENIPVVEGDPTRLHQVLTNLVANGLNHGGPEAEVNIQVETADDKVKILVIDNGVGMSKEDAEHIFERFYRADTSRSRASGGSGLGLAITKSLVEGHGGTITVDSELGKGTVFSIILPAAE

SEQ ID NO: 33 - protein, 458 aa, Corynebacterium crenatum
IGPIRAAGRAIPLRTRIILIVVGIAGLGLLVNAIAVSSLMREVSYTRMDQELETSMGTWAHNVELFNFDGVRQGPPSDYYVAKVFPDGSSIIFNDAQSAPDLAETTIGTGPHTVDAASGSASNTPWRVMAEKNGDIITVVGKSMGRETNLLYRLVMVQMIIGALILVAILITSLFLVRRSLRPLREVEETATRIAGGDLDRRVPQWPMTTEVGQLSNALNIMLEQLQASILSAQQKEAQMRRFVGDASHELRTPLTSVKGFTELYSSGATDDANWVMSKIGGEAQRMSVLVEDLLSLTRAEGQQMEKHRVDVLELALAVRGSMRAAWPDRTVNVSNKAASIPVVEGDPTRLHQVLTNLVANGLNHGGPDAEVSIEINTDGQNVRILVADNGVGMSEEDAQHIFERFYRADSSRSRASGGSGLGLAITKSLVEGHGGTVTVDSVQGEGTVFTITLPAVS

SEQ ID NO: 34 - protein, 471 aa, Corynebacterium efficiens
MTAPENPHAQVTPVGRFRQAARGVPLRTRIILLVVGIAGLGLLVNAIAVSSLMREVSYSRMDQELESAMNSWAQTAELFGSITLGPPSDYYVVRIFPDGSHMVFNQSDSAPDLGETTIGIGPHTASAAPGSSSSVPWRVIAISDNGTITVVGKSLAPESMLLYRLVIVQLVIGMLIVVAILLSSLYLVNRSLRPLREVEKTAKSIAGGDLDRRVPQWPMTTEVGQLANALNIMLEQLQASILSAQEKESQMRRFVGDASHELRTPLTSVKGYSELYHSGATRDADWVLSKISGEAQRMSVLVEDLLSLTRAEGQQMEKRPVDVLELSLSVASSMRAAWPERSITVVNKTGSLPVVEGDATRLHQVLTNLVNNGLNHGGPDASVEIEISAEGGSVLVRVVDDGVGMTAEDAQHIFERFYRTDTSRSRASGGSGLGLAITKSLVEGHRGTITVDSEVGEGTVFTITLPSRMED

SEQ ID NO: 35 - DNA, 708 bp, Corynebacterium glutamicum
atggacaaccagtctgacggacaaatccgcgtactcgtcgttgatgacgagccaaacatcgtcgagctgctcaccgtaagccttaaattccaaggcttcgcagtgatgaccgccaacgatggcaatgaagccctgaagattgctcgtgagttccgtccagacgcatacatcctcgatgtcatgatgccaggaatggacggcttcgagctgctgaccaagctgcgcggcgaaggccttgacagcccagttctgtacctcaccgcaaaggatgccgtggagcaccgcatccacggcctgaccatcggcgctgacgactacgtgaccaagcctttctccctggaagaagtaatcacccgcctgcgcgtgattcttcgtcgcggtggagcagttgaagaagacacctcaacttccctgcagtacgcagacctcaccctcaacgatgaaacccacgaggtcaccaaggctggcgaactgatcgatctttccccaactgaattcaacctcctgcgctacctcatgctcaacgctgaagtggtgctgtccaaggcaaagatcctggataacgtgtggcactacgattttggtggcgacggcaacgtcgtggaatcctacatctcctacctgcgccgcaaggtggacacccaggatccgcagctaattcagactgttcgtggcgttggatatgttctgcgcaccccacgtagctaa

SEQ ID NO: 36 - protein, 235 aa, Corynebacterium glutamicum
MDNQSDGQIRVLVVDDEPNIVELLTVSLKFQGFAVMTANDGNEALKIAREFRPDAYILDVMMPGMDGFELLTKLRGEGLDSPVLYLTAKDAVEHRIHGLTIGADDYVTKPFSLEEVITRLRVILRRGGAVEEDTSTSLQYADLTLNDETHEVTKAGELIDLSPTEFNLLRYLMLNAEVVLSKAKILDNVWHYDFGGDGNVVESYISYLRRKVDTQDPQLIQTVRGVGYVLRTPRS

SEQ ID NO: 37 - DNA, 1500 bp, Corynebacterium glutamicum
atgtttaacaaccgtatccgcactgcagctctcgctggtgcaatcgcaatctccaccgcagcttccggcgtagctatcccagcattcgctcaggagaccaacccaaccttcaacatcaacaacggcttcaacgatgctgatggatccaccatccagccagttgagccagttaaccacaccgaggaaaccctccgcgacctgactgactccaccggcgcttacctggaagagttccagtacggcaacgttgaggaaatcgttgaagcatacctgcaggttcaggcttccgcagacggattcgatccttctgagcaggctgcttacgaggctttcgaggctgctcgcgttcgtgcatcccaggagctcgcggcttccgctgagaccatcactaagacccgcgagtccgttgcttacgcactcaaggctgaccgcgaagctaccgcagctttcgaggcttacctcagcgctcttcgtcaggtttcagtcatcaacgatctgatcgctgatgctaacgccaagaacaagactgactttgcagagatcgagctctacgatgttctttacaccgacgccgacatctctggcgatgctccacttcttgctcctgcatacaaggagctgaaggaccttcaggctgaggttgacgcagacttcgagtggttgggcgagttcgcaattgataacaatgaagacaactacgtcattcgtactcacatccctgctgtagaggcactcaaggcagcgatcgattcactggtcgacaccgttgagccacttcgtgcagacgctatcgctaagaacatcgaggctcagaagtctgacgttctggttccccagctcttcctcgagcgtgcaactgcacagcgcgacaccctgcgtgttgtagaggcaatcttctctacctctgctcgttacgttgaactctacgagaacgtcgagaacgttaacgttgagaacaagacccttcgccagcactactcttccctgatccctaacctcttcatcgcagcggttggcaacatcaacgagctcaacaatgcagatcaggctgcacgtgagctcttcctcgattgggacaccgacctcaccaccaacgatgaggacgaagcttactaccaggctaagctcgacttcgctatcgagacctacgcaaagatcctgatcaacggtgaagtttggcaggagccactcgcttacgtccagaacctggatgcaggcgcacgtcaggaagcagctgaccgcgaagcagagcgcgcagctgacgcagcataccgcgctgagcagctccgcatcgctcaggaagcagctgacgctcagaaggctctcgctgaggctcttgctaatgcaggcaacaacgacaacggtggcgacaactcctccgacgacaagggaaccggttcttccgacatcggaacctggggacctttcgcagcaattgcagctatcatcgcagcaatcgcagctatcttcccattcctctccggtatcgttaagttctaa

SEQ ID NO: 38 - protein, 499 aa, Corynebacterium glutamicum
MFNNRIRTAALAGAIAISTAASGVAIPAFAQETNPTFNINNGFNDADGSTIQPVEPVNHTEETLRDLTDSTGAYLEEFQYGNVEEIVEAYLQVQASADGFDPSEQAAYEAFEAARVRASQELAASAETITKTRESVAYALKADREATAAFEAYLSALRQVSVINDLIADANAKNKTDFAEIELYDVLYTDADISGDAPLLAPAYKELKDLQAEVDADFEWLGEFAIDNNEDNYVIRTHIPAVEALKAAIDSLVDTVEPLRADAIAKNIEAQKSDVLVPQLFLERATAQRDTLRVVEAIFSTSARYVELYENVENVNVENKTLRQHYSSLIPNLFIAAVGNINELNNADQAARELFLDWDTDLTTNDEDEAYYQAKLDFAIETYAKILINGEVWQEPLAYVQNLDAGARQEAADREAERAADAAYRAEQLRIAQEAADAQKALAEALANAGNNDNGGDNSSDDKGTGSSDIGTWGPFAAIAAIIAAIAAIFPFLSGIVKF

SEQ ID NO: 39 - DNA, 318 bp, Corynebacterium glutamicum
atgtccctcggaccatgggaaattggaatcattgtcctgctgatcatcgtgctgttcggcgcgaagaagctgcctgatgcagctcgttccatcggccgttccatgcgcatcttcaagtctgaagtcaaagaaatgaacaaggacggcgataccccagaacaacagcagcagcctcagcagcagattgcgcccaaccagatcgaggctcctcagccaaactttgagcagcactaccagggacagcaggttcagcagcctcagaaccctcagacccctgactaccgtcagaactacgaggatccaaaccgcacctcttaa

SEQ ID NO: 40 - protein, 105 aa, Corynebacterium glutamicum
MSLGPWEIGIIVLLIIVLFGAKKLPDAARSIGRSMRIFKSEVKEMNKDGDTPEQQQQPQQQIAPNQIEAPQPNFEQHYQGQQVQQPQNPQTPDYRQNYEDPNRTS

SEQ ID NO: 41 - DNA, 471 bp, Corynebacterium glutamicum
atgttttctagcgtgggttggggagagatcttcctcttagtcgttgtgggccttgttgtcatcggcccggaacggttgcctcgtttgatccaggacgcacgcgctgcgctgctcgctgcacgtaccgctatcgacaatgcaaagcagtcgttggacagtgattttggttcggaatttgatgaaatccgaaagccactaacccaggttgcacagtacagccggatgagccccaagacggccatcactaaggcgttatttgataatgattcctcgttcctggatgactttgatccaaagaagatcatggccgaaggaacagaaggcgaagctcagcgcaacaagcaggcagctgacaacaatgcgaatgtggtggaacgtccagctgatggttccaccgcacgcccaacgcaaaacgatccaaaagacggcccgaattactcaggtggcgtctcttggaccgatattatttag

SEQ ID NO: 42 - protein, 156 aa, Corynebacterium glutamicum
MFSSVGWGEIFLLVVVGLVVIGPERLPRLIQDARAALLAARTAIDNAKQSLDSDFGSEFDEIRKPLTQVAQYSRMSPKTAITKALFDNDSSFLDDFDPKKIMAEGTEGEAQRNKQAADNNANVVERPADGSTARPTQNDPKDGPNYSGGVSWTDII

SEQ ID NO: 43 - DNA, 945 bp, Corynebacterium glutamicum
atgtccattgttgagcacatcaaagagtttcgacgccgacttcttatcgctctggcgggcatcctcgtgggcaccattatcggctttatttggtacgatttctcattttggcagatccccactttgggcgagctgctgagggatccgtactgttctctgcctgctgaatcccgctgggccatgagcgactcagaggaatgtcgactgctcgcaaccggcccgtttgatccattcatgcttcgccttaaagtagcggcgttggtgggtatggttcttggctcacccgtgtggctgagccagctgtggggctttatcaccccaggtttgatgaagaatgagcgccgttacaccgcaatcttcgtcacgattgctgttgtgctgtttgtcggcggtgctgttcttgcgtacttcgtcgttgcatatggtttggagttcctccttaccattggtggagacacccaggcagcggccctgactggtgataagtacttcggattcttgctcgcgttgttggcgattttcggcgtgagcttcgaagttccactggtgatcggcatgctcaacattgtgggtatcttgccttacgatgccattaaagataagcgacgcatgatcatcatgattttgttcgtgttcgctgctttcatgacacccggccaggatcctttcaccatgttggtgttggcgctttcactcaccgttctggtagagcttgccctgcagttctgtcgtttcaacgacaaacgccgggacaagaagcgcccagaatggcttgatggcgatgacctctctgcatcaccactggatacttctgctggtggagaagatgctccaagcccagtcgaaaccccagaggcggtggagccttcgcggatgctgaacccaagtggggaggcgtcgataagctataaacccgggcgcgccgacttcggtgacgtgctctag

SEQ ID NO: 44 - protein, 314 aa, Corynebacterium glutamicum
MSIVEHIKEFRRRLLIALAGILVGTIIGFIWYDFSFWQIPTLGELLRDPYCSLPAESRWAMSDSEECRLLATGPFDPFMLRLKVAALVGMVLGSPVWLSQLWGFITPGLMKNERRYTAIFVTIAVVLFVGGAVLAYFVVAYGLEFLLTIGGDTQAAALTGDKYFGFLLALLAIFGVSFEVPLVIGMLNIVGILPYDAIKDKRRMIIMILFVFAAFMTPGQDPFTMLVLALSLTVLVELALQFCRFNDKRRDKKRPEWLDGDDLSASPLDTSAGGEDAPSPVETPEAVEPSRMLNPSGEASISYKPGRADFGDVL

SEQ ID NO: 45 - protein, 5 aa, Artificial Sequence
  Twin-Arginine Motif
  misc_feature (3)..(5): Xaa can be any naturally occurring amino acid
RRXXX

SEQ ID NO: 46 - protein, 39 aa, Escherichia coli
MNNNDLFQASRRRFLAQLGGLTVAGMLGPSLLTPRRATA

SEQ ID NO: 47 - protein, 10 aa, Escherichia coli
GMLGPSLLTP

SEQ ID NO: 48 - protein, 10 aa, Streptomyces mobaraense
DNGAGEETKS

SEQ ID NO: 49 - protein, 10 aa, Chryseobacterium proteolyticum
DSNGNQEING

SEQ ID NO: 50 - protein, 10 aa, Unknown
  MULTISPECIES
HPETLVKVKD

SEQ ID NO: 51 - protein, 10 aa, Escherichia coli
ASDKIIHLTD

SEQ ID NO: 52 - protein, 10 aa, Corynebacterium glutamicum
APDSDWDRLA

SEQ ID NO: 53 - protein, 10 aa, Homo sapiens
MSFSGKYQLQ

SEQ ID NO: 54 - protein, 60 aa, Escherichia coli
MNNNDLFQASRRRFLAQLGGLTVAGMLGPSLLTPRRATADNGAGEETKSYAETYRLTADD

SEQ ID NO: 55 - protein, 60 aa, Escherichia coli
MNNNDLFQASRRRFLAQLGGLTVAGMLGPSLLTPRRATADSNGNQEINGKEKLSVNDSKL

SEQ ID NO: 56 - protein, 59 aa, Escherichia coli
MNNNDLFQASRRRFLAQLGGLTVAGMLGPSLLTPRRATAHPETLVKVKDAEDQLGARVG

SEQ ID NO: 57 - protein, 59 aa, Escherichia coli
MNNNDLFQASRRRFLAQLGGLTVAGMLGPSLLTPRRATAASDKIIHLTDDSFDTDVLKA

SEQ ID NO: 58 - protein, 59 aa, Escherichia coli
MNNNDLFQASRRRFLAQLGGLTVAGMLGPSLLTPRRATAMSFSGKYQLQSQENFEAFMK

SEQ ID NO: 59 - protein, 59 aa, Corynebacterium glutamicum
MRGKLFMGRHSTKTSSAFTKLAASTIAFGAAATIMAPSASAAPDSDWDRLAQCESGGNW

(19) United States
(12) Patent Application Publication
(10) Pub. No.: US 2021/0246479 A1
(43) Pub. Date: Aug. 12, 2021

(54) Method for Secretory Production of Protein

(71) Applicant: AJINOMOTO CO., INC., Tokyo (JP)

(72) Inventor: Yoshihiko Matsuda, Kanagawa (JP)

(73) Assignee: AJINOMOTO CO., INC., Tokyo (JP)

(21) Appl. No.: 17/236,370

(22) Filed: Apr. 21, 2021

Related U.S. Application Data
(63) Continuation of application No. PCT/JP2019/042069, filed on Oct. 25, 2019.

(30) Foreign Application Priority Data
Oct. 25, 2018 (JP) 2018-200673

(51) Int. Cl. C12P 21/02; C12N 15/77; C12N 15/62

(52) U.S. Cl. CPC C12P 21/02; C12N 15/62; C12N 15/77

(57) ABSTRACT

A novel technique for reducing the mis-cleavage of the TorA signal peptide, and thereby a method for efficient secretory production of a heterologous protein by a coryneform bacterium using a TorA signal peptide is provided. A coryneform bacterium having an ability of secretory production of a heterologous protein using a TorA signal peptide and has been modified so that the activity of a LepB protein is increased is cultured to produce the heterologous protein by secretory production.

This application is a Continuation of, and claims priority under 35 U.S.C. § 120 to, International Application No. PCT/JP2019/042069, filed Oct. 25, 2019, and claims priority therethrough under 35 U.S.C. § 119 to Japanese Patent Application No. 2018-200673, filed Oct. 25, 2018, the entireties of which are incorporated by reference herein. Also, the Sequence Listing filed electronically herewith is hereby incorporated by reference (File name: 2021-04-21T_US-626_Seq List; File size: 61 KB; Date recorded: Apr. 21, 2021).

### BACKGROUND

### Technical Field

The present invention relates to a method for secretory production of a heterologous protein.

### Background Art

Methods for secretory production of heterologous proteins, for example, methods using microorganisms such as coryneform bacteria as expression hosts, have been previously reported. For the purpose of efficient secretory production of heterologous proteins, for example, signal peptides (also referred to as “signal sequences”) can be used. That is, by expressing a heterologous protein fused at the N-terminus with a signal peptide corresponding to a protein secretion system possessed by a host, the heterologous protein can be efficiently produced by secretory production.

A general protein secretion pathway is a pathway called “Sec system”, which is widely present from prokaryotes to eukaryotes. In secretory production of heterologous proteins by coryneform bacteria using the Sec system, secretory production of a heterologous protein can be improved by, for example, inserting the amino acid sequence Gln-Glu-Thr or Ala-Glu-Thr between a signal peptide and the heterologous protein.

Meanwhile, a protein secretion pathway completely different from the Sec system has recently been found in thylakoid membranes of chloroplasts of plant cells (EMBO J., 14, 2715-2722(1995)). This novel secretory pathway has been named the “Tat system” (Twin-Arginine Translocation system) because an arginine-arginine sequence is commonly present in the signal peptide of the secreted protein (EMBO J., 14, 2715-2722(1995)). It is known that proteins are secreted by the Sec system before forming a higher-order structure, while proteins are secreted by the Tat system through a cell membrane after forming a higher-order structure in the cell (J. Biol. Chem., 25;273(52), 34868-74(1998)). Also for coryneform bacteria, secretory production of proteins utilizing a Tat-dependent signal peptide such as a TorA signal peptide has been reported (WO2013/118544, Japanese Patent No. 4730302, etc.).

Signal peptides are generally cleaved at the C-terminus by a signal peptidase and removed when the proteins are secreted out of cells. However, in secretory production of proteins, sometimes problems can occur and signal peptides are not cleaved, and as a result proteins accumulate in the cells. In such a case, it has been previously reported that secretion efficiency of proteins can be improved by overexpressing a signal peptidase such as LepB protein (EP444759, EP974654, WO2004/007740, DE102004035543, KR101077783, CN104611317, and Biochem Biophys Res Commun. 1999 255(2):444-50, Biotechnol Bioeng. 2005 91(5):616-21, J Microbiol Biotechnol. 2007 Aug;17(8):1316-23, J Biol Chem. 2011 Dec 23;286(51):43601-10, and J Appl Microbiol. 2016 Sep;121(3):704-12).

However, the phenomenon of a signal peptide being cleaved within the peptide instead of at the C-terminus, and as a result, proteins are secreted out of cells while having a C-terminal-side partial sequence of the signal peptide.

### SUMMARY

In the secretory production of a heterologous protein by a coryneform bacterium using a TorA signal peptide, it has been found that the TorA signal peptide is not cleaved at the correct position and as a result, mis-cleavage of C-terminal side 15 residues occurs. Described herein is a novel technique for reducing the mis-cleavage of the TorA signal peptide, and thereby to provide a method for secretory production of a heterologous protein by a coryneform bacterium using the TorA signal peptide.

Specifically, the mis-cleavage of the TorA signal peptide in secretory production of a heterologous protein by a coryneform bacterium using the TorA signal peptide can be reduced by modifying the coryneform bacterium so that the activity of a LepB protein is increased.

It is an aspect of the present invention to provide a method for producing a heterologous protein comprising: culturing a coryneform bacterium having a genetic construct for secretory expression of the heterologous protein; and collecting the heterologous protein produced by secretory production, wherein the coryneform bacterium has been modified so that the activity of a LepB protein is increased, wherein the genetic construct comprises, in the direction from 5′ to 3′, a promoter sequence that functions in the coryneform bacterium, a nucleic acid sequence encoding a TorA signal peptide, and a nucleic acid sequence encoding the heterologous protein, and wherein the heterologous protein is expressed as a fusion protein with the TorA signal peptide.

It is a further aspect of the present invention to provide the method described above, wherein the LepB protein is selected from the group consisting of: (a) a protein comprising the amino acid sequence of SEQ ID NO: 2; (b) a protein comprising the amino acid sequence of SEQ ID NO: 2, but which includes substitution, deletion, insertion, and/or addition of 1 to 10 amino acid residues, wherein said protein has a signal peptidase activity for the TorA signal peptide; and (c) a protein comprising an amino acid sequence having an identity of 90% or higher to the amino acid sequence of SEQ ID NO: 2, wherein said protein has a signal peptidase activity for the TorA signal peptide.

It is a further aspect of the present invention to provide the method described above, wherein the activity of the LepB protein is increased by increasing the expression of a lepB gene.

It is a further aspect of the present invention to provide the method described above, wherein the expression of the lepB gene is increased by increasing the copy number of lepB gene and/or modifying an expression control sequence of the lepB gene.

It is a further aspect of the present invention to provide the method described above, wherein the TorA signal peptide is a peptide selected from the group consisting of: (a) a peptide comprising the amino acid sequence of SEQ ID NO: 46; (b) a peptide comprising the amino acid sequence of SEQ ID NO: 46, but which includes substitution, deletion, insertion, and/or addition of 1 to 3 amino acid residues, wherein said protein has a function as a Tat-dependent signal peptide; and (c) a peptide comprising an amino acid sequence having an identity of 90% or higher to the amino acid sequence of SEQ ID NO: 46, wherein said protein has a function as a Tat-dependent signal peptide.

It is a further aspect of the present invention to provide the method described above, wherein the TorA signal peptide consists of the amino acid sequence of SEQ ID NO: 46.

It is a further aspect of the present invention to provide the method described above, wherein the heterologous protein produced by secretory production is a heterologous protein from which the TorA signal peptide has been completely removed.

It is a further aspect of the present invention to provide the method described above, wherein the coryneform bacterium has been further modified so as to harbor a phoS gene encoding a mutant PhoS protein.

It is a further aspect of the present invention to provide the method described above, wherein the mutation is a mutation of replacing an amino acid residue corresponding to the tryptophan residue at position 302 in SEQ ID NO: 29 with an amino acid residue other than aromatic amino acid and histidine residues.

It is a further aspect of the present invention to provide the method described above, wherein the amino acid residue other than aromatic amino acid and a histidine residue is selected from the group consisting of a lysine residue, alanine residue, valine residue, serine residue, cysteine residue, methionine residue, aspartic acid residue, and asparagine residue.

It is a further aspect of the present invention to provide the method described above, wherein the wild-type PhoS protein is a protein defined in (a), (b), or (c) mentioned below: (a) a protein comprising any of the amino acid sequences of SEQ ID NOS: 29 to 34; (b) a protein comprising any of the amino acid sequences of SEQ ID NOS: 29 to 34, but which includes substitution, deletion, insertion, and/or addition of 1 to 10 amino acid residues, wherein said protein has a function as a sensor kinase of a PhoRS system; and (c) a protein comprising an amino acid sequence having an identity of 90% or higher to any of the amino acid sequences of SEQ ID NOS: 29 to 34, wherein said protein has a function as a sensor kinase of a PhoRS system.

It is a further aspect of the present invention to provide the method described above, wherein the coryneform bacterium has been further modified so that the expression of one or more of the genes encoding a Tat secretion system is increased as compared with a non-modified strain.

It is a further aspect of the present invention to provide the method described above, wherein the genes encoding a Tat secretion system consists of a tatA gene, tatB gene, tatC gene, and tatE gene.

It is a further aspect of the present invention to provide the method described above, wherein the coryneform bacterium belongs to the genus Corynebacterium.

It is a further aspect of the present invention to provide the method described above, wherein the coryneform bacterium is Corynebacterium glutamicum.

It is a further aspect of the present invention to provide the method described above, wherein the Corynebacterium glutamicum is derived from Corynebacterium glutamicum AJ12036 (FERM BP-734) or Corynebacterium glutamicum ATCC 13869.

It is a further aspect of the present invention to provide the method described above, wherein the number of molecules of a cell surface layer protein per coryneform bacterium is reduced as compared with a non-modified coryneform bacterium.

### BRIEF DESCRIPTION OF THE DRAWINGS

FIG. 1A-1D is a diagram showing the structures of vectors.

FIG. 2A-2B is a photograph showing the result of SDS-PAGE observed upon expressing PTG (transglutaminase comprising a pro-structure moiety) fused with TorA signal peptide in the C. glutamicum YDK010::phoS(W302C) strain and a lepB gene expression-enhanced strain thereof. The amino acid sequence in this FIG. is shown in SEQ ID NO: 54.

FIG. 3A-3B is a photograph showing the result of SDS-PAGE observed upon expressing PPG (protein glutaminase comprising a pro-structure moiety) fused with TorA signal peptide in the C. glutamicum YDK010::phoS(W302C) strain and a lepB gene expression-enhanced strain thereof. The amino acid sequence in this FIG. is shown in SEQ ID NO: 55.

FIG. 4A-4B is a photograph showing the result of SDS-PAGE observed upon expressing Bla (beta-lactamase) fused with TorA signal peptide in the C. glutamicum YDK010::phoS(W302C) strain and a lepB gene expression-enhanced strain thereof. The amino acid sequence in this FIG. is shown in SEQ ID NO: 56.

FIG. 5A-5B is a photograph showing the result of SDS-PAGE observed upon expressing Trx (thioredoxin) fused with TorA signal peptide in the C. glutamicum YDK010::phoS(W302C) strain and a lepB gene expression-enhanced strain thereof. The amino acid sequence in this FIG. is shown in SEQ ID NO: 57.

FIG. 6A-6B is a photograph showing the result of SDS-PAGE observed upon expressing LFABP (Liver-type fatty acid-binding protein) fused with TorA signal peptide in the C. glutamicum YDK010::phoS(W302C) strain and a lepB gene expression-enhanced strain thereof. The amino acid sequences in this FIG. are shown in SEQ ID NOS: 58 and 59.

FIG. 7A-7B is a photograph showing the result of SDS-PAGE observed upon expressing PTG (transglutaminase comprising a pro-structure moiety) fused with SlpA signal peptide in the C. glutamicum YDK010::phoS(W302C) strain and a lepB gene expression-enhanced strain thereof. The amino acid sequence in this FIG. is shown in SEQ ID NO: 54.

### DETAILED DESCRIPTION

The method as described herein is a method for producing a heterologous protein, including the steps of culturing a coryneform bacterium having a genetic construct for secretory expression of the heterologous protein, and collecting the heterologous protein produced by secretory production, wherein the coryneform bacterium has been modified so that the activity of a LepB protein is increased, and wherein a TorA signal peptide is used in secretory production of the heterologous protein.

In the method, mis-cleavage of a TorA signal peptide can be reduced, and as a result a heterologous protein in a completely-cleaved form can be produced by secretory production. That is, the phrase “heterologous protein” to be produced by secretory production in the method as described herein refers to a heterologous protein in a completely-cleaved form, unless otherwise stated. The phrase “heterologous protein in a completely-cleaved form” refers to a heterologous protein from which the TorA signal peptide has been completely removed, i.e. a heterologous protein not having the TorA signal peptide at all.

<1>Coryneform Bacterium used for the Method

The coryneform bacterium used for the method is a coryneform bacterium having a genetic construct for secretory expression of a heterologous protein, which has been modified so that the activity of a LepB protein is increased. The coryneform bacterium used for the method is also referred to as “bacterium as described herein” or “coryneform bacterium as described herein”. Furthermore, the genetic construct for secretory expression of a heterologous protein harbored by the bacterium is also referred to as “genetic construct as described herein”. Furthermore, the bacterium or a strain used for constructing the same is also referred to as “host”.

<1-1>Coryneform Bacterium Having Ability of Secretory Production of Heterologous protein

The coryneform bacterium as described herein has an ability of secretory production of a heterologous protein, specifically, a heterologous protein in a completely-cleaved form. The coryneform bacterium as described herein has an ability of secretory production of a heterologous protein, relying at least on having the genetic construct for secretory expression of the heterologous protein (genetic construct used for the method). Specifically, the coryneform bacterium may have an ability of secretory production of a heterologous protein, relying on having the genetic construct for secretory expression of the heterologous protein, or relying on a combination of having the genetic construct for secretory expression of the heterologous protein and other characteristic(s).

The expression that a protein is “secreted” means that the protein is transported out of a bacterial cell (extracellularly transported). Examples of a position outside of a bacterial cell (outside of a cell) include a medium and a cell surface layer. That is, molecules of the secreted protein may be present, for example, in the medium, in the cell surface layer, or in both of the medium and the cell surface layer. That is, the expression that a protein is “secreted” is not limited when all the molecules of the protein eventually are present in the medium in completely free forms, but also includes, for example, when all the molecules of the protein are present in the cell surface layer, and also when some of the molecules of the protein are present in the medium and the remaining molecules of the protein are present in the cell surface layer.

That is, the phrase “ability to produce a heterologous protein by secretory production” refers to an ability of the bacterium as described herein to secrete the heterologous protein into a medium and/or a cell surface layer, and accumulate it there to such an extent that the heterologous protein can be collected from the medium and/or the cell surface layer, when the bacterium is cultured in the medium. The accumulation amount may be, for example, in terms of the accumulation amount in the medium, 10 μg/L or more, 1 mg/L or more, 100 mg/L or more, or 1 g/L or more. Also, the accumulation amount may be, for example, in terms of the accumulation amount in the cell surface layer, such an amount that if the heterologous protein in the cell surface layer is collected and suspended in a liquid of the same volume as the medium, the concentration of the heterologous protein in the suspension is 10 μg/L or more, 1 mg/L or more, or 100 mg/L or more. In addition, the term “protein” in reference to those produced by secretory production refers also to peptides, such as oligopeptides and polypeptides.

The term “heterologous protein” refers to an exogenous protein relative to a coryneform bacterium that expresses and secretes that protein. The heterologous protein may be, for example, a protein derived from a microorganism, a protein derived from a plant, a protein derived from an animal, a protein derived from a virus, or even a protein of which the amino acid sequence is artificially designed. The heterologous protein may particularly be a derived from human. The heterologous protein may be a monomeric protein or a multimeric protein. The term “multimeric protein” refers to a protein that contains two or more subunits. In the multimer, the subunits may be linked by covalent bonds such as disulfide bonds, linked by non-covalent bonds such as hydrogen bonds and hydrophobic interaction, or linked by a combination thereof. The multimer can include one or more intermolecular disulfide bonds. The multimer may be a homo-multimer having a single kind of subunit, or may be a hetero-multimer having two or more kinds of subunits. When the multimeric protein is a hetero-multimer, it is sufficient that at least one subunit is a heterologous protein. That is, all the subunits may be heterologous, or only some of the subunits may be heterologous. Although the heterologous protein may be a secretory protein in nature, or may be a non-secretory protein in nature, it can be a secretory protein in nature. Furthermore, the heterologous protein may be a Tat-dependent secretory protein in nature, or may be a Sec-dependent secretory protein in nature. Specific examples of the “heterologous protein” will be mentioned later.

The heterologous protein to be produced may include single kind of protein, or two or more kinds of proteins. Moreover, when the heterologous protein is a hetero-multimer, only one kind of subunit may be produced, or two or more kinds of subunits may be produced. That is, the term “secretory production of a heterologous protein” includes secretory production of all the subunits constituting an objective heterologous protein, as well as secretory production of only a part of the subunits constituting an objective heterologous protein.

Coryneform bacteria are aerobic gram-positive bacilli. Examples of the coryneform bacteria include Corynebacterium bacteria, Brevibacterium bacteria, Microbacterium bacteria, and so forth. Advantages of use of the coryneform bacteria include that they inherently secrete an extremely small amount of proteins out of cells compared with fungi, yeasts, Bacillus bacteria, etc., which are conventionally used for secretory production of proteins, and therefore the purification process of a heterologous protein produced by secretory production is expected to be simplified or eliminated, that they can grow well in a simple medium containing a saccharide, ammonia, mineral salts, etc., and therefore they are excellent in view of cost of medium, culture method, and culture productivity, and so forth.

Specific examples of coryneform bacteria include the following species:

Corynebacterium acetoacidophilum

Corynebacterium acetoglutamicum

Corynebacterium alkanolyticum

Corynebacterium callunae

Corynebacterium crenatum

Corynebacterium glutamicum

Corynebacterium lilium

Corynebacterium melassecola

Corynebacterium thermoaminogenes (Corynebacterium efficiens)

Corynebacterium herculis

Brevibacterium divaricatum (Corynebacterium glutamicum)

Brevibacterium flavum (Corynebacterium glutamicum)

Brevibacterium immariophilum

Brevibacterium lactofermentum (Corynebacterium glutamicum)

Brevibacterium roseum

Brevibacterium saccharolyticum

Brevibacterium thiogenitalis

Corynebacterium ammoniagenes (Corynebacterium stationis)

Brevibacterium album

Brevibacterium cerinum

Microbacterium ammoniaphilum

Specific examples of coryneform bacteria include the following strains:

Corynebacterium acetoacidophilum ATCC 13870

Corynebacterium acetoglutamicum ATCC 15806

Corynebacterium alkanolyticum ATCC 21511

Corynebacterium callunae ATCC 15991

Corynebacterium crenatum AS1.542

Corynebacterium glutamicum ATCC 13020, ATCC 13032, ATCC 13060, ATCC 13869, FERM BP-734

Corynebacterium lilium ATCC 15990

Corynebacterium melassecola ATCC 17965

Corynebacterium thermoaminogenes (Corynebacterium efficiens) AJ12340 (FERM BP-1539)

Corynebacterium herculis ATCC 13868

Brevibacterium divaricatum (Corynebacterium glutamicum) ATCC 14020

Brevibacterium flavum (Corynebacterium glutamicum) ATCC 13826, ATCC 14067, AJ12418 (FERM BP-2205)

Brevibacterium immariophilum ATCC 14068

Brevibacterium lactofermentum (Corynebacterium glutamicum) ATCC 13869

Brevibacterium roseum ATCC 13825

Brevibacterium saccharolyticum ATCC 14066

Brevibacterium thiogenitalis ATCC 19240

Corynebacterium ammoniagenes (Corynebacterium stationis) ATCC 6871, ATCC 6872

Brevibacterium album ATCC 15111

Brevibacterium cerinum ATCC 15112

Microbacterium ammoniaphilum ATCC 15354

The Corynebacterium bacteria include bacteria that had previously been classified into the genus Brevibacterium, but have been united into the genus Corynebacterium (Int. J. Syst. Bacteriol., 41, 255 (1991)). Moreover, Corynebacterium stationis includes bacteria that had previously been classified as Corynebacterium ammoniagenes, but are presently re-classified into Corynebacterium stationis on the basis of nucleotide sequence analysis of 16S rRNA etc. (Int. J. Syst. Evol. Microbiol., 60, 874-879 (2010)).

These strains are available from, for example, the American Type Culture Collection (Address: 10801 University Boulevard, Manassas, Va. 20110, United States of America). That is, registration numbers are assigned to the respective strains, and the strains can be ordered by using these registration numbers (refer to atcc.org). The registration numbers of the strains are listed in the catalogue of the American Type Culture Collection. These strains can also be obtained from, for example, the depositories at which the strains were deposited.

In particular, the Corynebacterium glutamicum (C. glutamicum) AJ12036 strain (FERM BP-734), which was isolated as a streptomycin (Sm) resistant mutant strain from a wild-type strain C. glutamicum ATCC 13869 is predicted to have a mutation in a gene responsible for a function involved in secretion of proteins, and shows an extremely high secretory production ability for proteins as high as about 2 to 3 times in terms of accumulation amount of proteins under optimum culture conditions, compared with the parent strain (wild-type strain), and therefore it is preferred as a host bacterium (WO2002/081694). The AJ12036 strain was originally deposited at the Fermentation Research Institute, Agency of Industrial Science and Technology (currently, independent administrative agency, National Institute of Technology and Evaluation, International Patent Organism Depositary, #120, 2-5-8 Kazusakamatari, Kisarazu-shi, Chiba-ken, 292-0818, Japan) on Mar. 26, 1984 as an international deposit, and assigned an accession number of FERM BP-734.

Corynebacterium thermoaminogenes AJ12340 (FERM BP-1539) was originally deposited at the Fermentation Research Institute, Agency of Industrial Science and Technology (currently, independent administrative agency, National Institute of Technology and Evaluation, International Patent Organism Depositary, #120, 2-5-8 Kazusakamatari, Kisarazu-shi, Chiba-ken, 292-0818, Japan) on Mar. 13, 1987 as an international deposit, and assigned an accession number of FERM BP-1539. Brevibacterium flavum AJ12418 (FERM BP-2205) was originally deposited at the Fermentation Research Institute, Agency of Industrial Science and Technology (currently, independent administrative agency, National Institute of Technology and Evaluation, International Patent Organism Depositary, #120, 2-5-8 Kazusakamatari, Kisarazu-shi, Chiba-ken, 292-0818, Japan) on Dec. 24, 1988 as an international deposit, and assigned an accession number of FERM BP-2205.

Moreover, a strain having an enhanced ability to produce a protein by secretory production may be a coryneform bacterium as mentioned above as a parent strain by using a mutagenesis method or a genetic recombination method, and used as a host. For example, after a parent strain is treated with ultraviolet irradiation or a chemical mutation agent such as N-methyl-N′-nitrosoguanidine, a strain having an enhanced ability to produce a protein by secretory production can be selected.

Furthermore, if a strain obtained by modifying such a strain as mentioned above so that it does not produce a cell surface layer protein is used as a host, purification of the heterologous protein secreted in the medium or on the cell surface layer becomes easy, and therefore it is particularly preferred. Such modification can be carried out by introducing a mutation into the coding region of the cell surface layer protein or an expression control region thereof, on the chromosome by mutagenesis or genetic recombination. Examples of coryneform bacterium modified so that it does not produce a cell surface layer protein include the C. glutamicum YDK010 strain (WO2002/081694), which is a cell surface layer protein PS2 deficient strain of the C. glutamicum AJ12036 strain (FERM BP-734).

A coryneform bacterium having an ability of secretory production of a heterologous protein can be obtained by introducing the genetic construct used for the method as described herein into such a coryneform bacterium as described above so as to make the bacterium harbor the genetic construct. That is, the bacterium may be, for example, a modified strain derived from such a coryneform bacterium as described above. The bacterium may be, specifically, for example, a modified strain derived from C. glutamicum AJ12036 (FERM BP-734) or a modified strain derived from C. glutamicum ATCC 13869. A modified strain derived from C. glutamicum AJ12036 (FERM BP-734) falls also within a modified strain derived from C. glutamicum ATCC 13869. The genetic construct used for the method and methods for introduction of the same will be described later.

<1-2>Increase in Activity of LepB Protein

The bacterium can be modified so that the activity of a LepB protein is increased. Specifically, the bacterium can be modified so that the activity of a LepB protein is increased as compared with a non-modified strain. More specifically, the bacterium can be modified so that the expression of a lepB gene is increased. By modifying a coryneform bacterium so that the activity of a LepB protein is increased, mis-cleavage of a TorA signal peptide in secretory production of a heterologous protein by the bacterium using the TorA signal peptide can be reduced. Examples of reduction in mis-cleavage of a TorA signal peptide include reduction in mis-cleavage rate of a TorA signal peptide. The phrase “mis-cleavage rate of a TorA signal peptide” refers to weight ratio of the secretory production amount of a heterologous protein in a mis-cleaved form to the total secretory production amount of the heterologous protein. The phrase “total secretory production amount of a heterologous protein” refers to the sum of the secretory production amount of a heterologous protein in a completely-cleaved form and the secretory production amount of the heterologous protein in a mis-cleaved form. The phrase “heterologous protein in a mis-cleaved form” refers to a heterologous protein from which the TorA signal peptide has been partially removed, i.e. a heterologous protein holding a C-terminal side partial sequence of the TorA signal peptide. Examples of the C-terminal side partial sequence of the TorA signal peptide include the amino acid sequence of C-terminal side 15 residues of the TorA signal peptide. Specific examples of the C-terminal side partial sequence of the TorA signal peptide include the amino acid sequence of positions 25-39 of SEQ ID NO: 46. The mis-cleavage rate of the TorA signal peptide in the method may be, for example, 3% or lower, 1% or lower, 0.5% or lower, 0.3% or lower, 0.1% or lower, or 0%. The mis-cleavage rate of the TorA signal peptide in the method may also be, for example, 50% or lower, 30% or lower, 10% or lower, 5% or lower, 3% or lower, 1% or lower, or 0% of the mis-cleavage rate of the TorA signal peptide observed when using a non-modified strain, which herein refers to a strain not modified so that the activity of a LepB protein is increased. Also, by modifying a coryneform bacterium so that the activity of a LepB protein is increased, it is expected that an ability of the bacterium to produce a heterologous protein, specifically a heterologous protein in a completely-cleaved form, by secretory production when using the TorA signal peptide can be improved, that is, secretory production of a heterologous protein, specifically a heterologous protein in a completely-cleaved form, by the bacterium using the TorA signal peptide can be increased.

The bacterium can be obtained by modifying a coryneform bacterium having an ability of secretory production of a heterologous protein so that the activity of a LepB protein is increased. The bacterium can also be obtained by modifying a coryneform bacterium so that the activity of a LepB protein is increased, and then imparting an ability of secretory production of a heterologous protein thereto. Modifications for constructing the bacterium can be performed in any order. A strain to be used for constructing the bacterium and before being modified so that the activity of a LepB protein is increased may or may not be able to produce a heterologous protein, on the assumption that the strain has the genetic construct for secretory expression of the heterologous protein. That is, the bacterium may also be, for example, a bacterium that has acquired an ability of secretory production of a heterologous protein due to being modified so that the activity of a LepB protein is increased. Specifically, for example, the bacterium may also be obtained from a strain that is not able to produce a heterologous protein by secretory production even when it has the genetic construct for secretory expression of the heterologous protein before it is modified so that the activity of a LepB protein is increased, which came to be able to produce the heterologous protein by secretory production due to being modified so that the activity of a LepB protein is increased.

Hereinafter, the LepB protein and the lepB gene encoding the same will be explained. The LepB protein is a signal peptidase. The phrase “signal peptidase” refers to a protein (enzyme) having an activity of catalyzing processing of a signal peptide. This activity is also referred to as “signal peptidase activity”. The phrase “processing of a signal peptide” refers to a reaction of removing the whole of a signal peptide from a protein having the signal peptide. The LepB protein at least has the signal peptidase activity for the TorA signal peptide, i.e. an activity of catalyzing processing of the TorA signal peptide.

Examples of the lepB gene and the LepB protein include those native to various organisms such as coryneform bacteria and bacteria of the family Enterobacteriaceae. The nucleotide sequences of lepB genes native to various organisms and the amino acid sequences of LepB proteins encoded by them can be obtained from, for example, public databases such as NCBI (National Center for Biotechnology Information). The nucleotide sequence of the lepB gene of C. glutamicum ATCC 13869 is shown as SEQ ID NO: 1, and the amino acid sequence of the LepB protein encoded by this gene is shown as SEQ ID NO: 2. That is, the lepB gene may be, for example, a gene having the nucleotide sequence shown as SEQ ID NO: 1. Also, the LepB protein may be, for example, a protein having the amino acid sequence shown as SEQ ID NO: 2.

The lepB gene may be a variant of any of the lepB genes exemplified above (such as a gene having the nucleotide sequence shown as SEQ ID NO: 1), so long as the original function thereof is maintained. Similarly, the LepB protein may be a variant of any of the LepB proteins exemplified above (such as a protein having the amino acid sequence shown as SEQ ID NO: 2), so long as the original function thereof is maintained. Such a variant is also referred to as “conservative variant”. The term “lepB gene” includes not only the lepB genes exemplified above, but also includes conservative variants thereof. Similarly, the term “LepB protein” includes not only the LepB proteins exemplified above, but also includes conservative variants thereof. Examples of the conservative variants include, for example, homologues and artificially modified versions of the lepB genes and LepB proteins exemplified above.

The expression “the original function is maintained” means that a variant of a gene or protein has a function (such as activity or property) corresponding to the function (such as activity or property) of the original gene or protein. That is, the expression “the original function is maintained” used for the lepB gene means that a variant of the gene encodes a protein that maintains the original function. Furthermore, the expression “the original function is maintained” used for the LepB protein means that a variant of the protein has the signal peptidase activity for the TorA signal peptide.

The signal peptidase activity can be measured by, for example, incubating the enzyme with a substrate, i.e. a protein having a signal peptide, and measuring the enzyme-dependent processing of the signal peptide. Processing of the signal peptide can be measured by, for example, using the molecular weight or the N-terminal amino acid sequence of the substrate after the incubation as an indicator.

Hereinafter, examples of the conservative variants will be explained.

Homologues of the lepB genes and homologues of the LepB proteins can be easily obtained from public databases by, for example, BLAST search or FASTA search using any of the nucleotide sequences of the lepB genes exemplified above or any of the amino acid sequences of the LepB proteins exemplified above as a query sequence. Furthermore, homologues of the lepB genes can be obtained by, for example, PCR using a chromosome of coryneform bacteria as the template, and oligonucleotides prepared on the basis of any of the nucleotide sequences of these known lepB genes as primers.

The lepB gene may be a gene encoding a protein having any of the amino acid sequences of the LepB proteins exemplified above (such as the amino acid sequence shown as SEQ ID NO: 2), but which includes substitution, deletion, insertion, and/or addition of one or several amino acid residues at one or several positions, so long as the original function thereof is maintained. Although the number meant by the term “one or several” mentioned above may differ depending on the positions of amino acid residues in the three-dimensional structure of the protein or the types of amino acid residues, specifically, it is, for example, 1 to 50, 1 to 40, 1 to 30, 1 to 20, 1 to 10, 1 to 5, or 1 to 3.

The aforementioned substitution, deletion, insertion, and/or addition of one or several amino acid residues is a conservative mutation that maintains the normal function of the protein. Typical examples of the conservative mutation are conservative substitutions. The conservative substitution is a mutation wherein substitution takes place mutually among Phe, Trp, and Tyr, if the substitution site is an aromatic amino acid; among Leu, Ile, and Val, if it is a hydrophobic amino acid; between Gln and Asn, if it is a polar amino acid; among Lys, Arg, and His, if it is a basic amino acid; between Asp and Glu, if it is an acidic amino acid; and between Ser and Thr, if it is an amino acid having a hydroxyl group. Examples of substitutions considered as conservative substitutions include, specifically, substitution of Ser or Thr for Ala, substitution of Gln, His, or Lys for Arg, substitution of Glu, Gln, Lys, His, or Asp for Asn, substitution of Asn, Glu, or Gln for Asp, substitution of Ser or Ala for Cys, substitution of Asn, Glu, Lys, His, Asp, or Arg for Gln, substitution of Gly, Asn, Gln, Lys, or Asp for Glu, substitution of Pro for Gly, substitution of Asn, Lys, Gln, Arg, or Tyr for His, substitution of Leu, Met, Val, or Phe for Ile, substitution of Ile, Met, Val, or Phe for Leu, substitution of Asn, Glu, Gln, His, or Arg for Lys, substitution of Ile, Leu, Val, or Phe for Met, substitution of Trp, Tyr, Met, Ile, or Leu for Phe, substitution of Thr or Ala for Ser, substitution of Ser or Ala for Thr, substitution of Phe or Tyr for Trp, substitution of His, Phe, or Trp for Tyr, and substitution of Met, Ile, or Leu for Val. Furthermore, such substitution, deletion, insertion, or addition of amino acid residues as mentioned above includes a naturally occurring mutation due to an individual difference, or a difference of species of the bacterium from which the gene is derived (mutant or variant).

The lepB gene may also be a gene encoding a protein having an amino acid sequence showing an identity of, for example, 80% or more, 90% or more, 95% or more, 97% or more, or 99% or more, to the total amino acid sequence of any of the amino acid sequences of the LepB proteins exemplified above (such as the amino acid sequence shown as SEQ ID NO: 2), so long as the original function thereof is maintained.

The lepB gene may also be DNA that is able to hybridize under stringent conditions with a complementary sequence of any of the nucleotide sequences of the lepB genes exemplified above (such as the nucleotide sequence shown as SEQ ID NO: 1), or with a probe that can be prepared from the complementary sequence, so long as the original function thereof is maintained. The term “stringent conditions” refers to conditions under which a so-called specific hybrid is formed, and a non-specific hybrid is not formed. Examples of the stringent conditions include those under which highly identical DNAs hybridize to each other, for example, DNAs not less than 80% identical, not less than 90% identical, not less than 95% identical, not less than 97% identical, or not less than 99% identical, hybridize to each other, and DNAs less identical than the above do not hybridize to each other, or conditions of washing of typical Southern hybridization, i.e., conditions of washing once, or 2 or 3 times, at a salt concentration and temperature corresponding to 19× SSC, 0.1% SDS at 60° C., 0.1× SSC, 0.1% SDS at 60° C., or 0.1× SSC, 0.1% SDS at 68° C.

The probe may be, for example, a part of a sequence that is complementary to the gene as described above. Such a probe can be prepared by PCR using oligonucleotides prepared on the basis of the nucleotide sequences of known genes as primers and a DNA fragment containing any of these nucleotide sequences as a template. As the probe, for example, a DNA fragment having a length of about 300 bp can be used. In such a case, the washing conditions of the hybridization may be, for example, 50° C., 2× SSC and 0.1% SDS.

Furthermore, the lepB gene may be a gene having a nucleotide sequence corresponding to any of the nucleotide sequences of the lepB genes exemplified above or conservative variants thereof in which any codon(s) is/are replaced with respective equivalent codon(s). For example, the lepB gene may have been modified so as to have optimal codons according to the codon usage frequency of the host to be used.

The term “identity” between amino acid sequences means an identity between the amino acid sequences calculated by blastp with default scoring parameters (i.e. Matrix, BLOSUM62; Gap Costs, Existence=11, Extension=1; Compositional Adjustments, Conditional compositional score matrix adjustment), unless otherwise stated. The term “identity” between nucleotide sequences means an identity between the nucleotide sequences calculated by blastn with default scoring parameters (i.e. Match/Mismatch Scores=1, −2; Gap Costs =Linear), unless otherwise stated.

The aforementioned descriptions concerning variants of the genes and proteins can also be similarly applied to any proteins such as PhoRS proteins, cell surface layer protein, Tat secretion system, and heterologous proteins to be produced by secretory production in the present invention, and genes encoding them.

<1-3>Other Characteristics

The bacterium may have desired characteristics, so long as it can produce a heterologous protein by secretory production. For example, the activity of a cell surface layer protein may have been reduced in the bacterium (WO2013/065869, WO2013/065772, WO2013/118544, and WO2013/062029). Also, the bacterium may have been modified so that the activity of a penicillin-binding protein is reduced (WO2013/065869). Also, the bacterium may have been modified so that the expression of a gene encoding a metallopeptidase is increased (WO2013/065772). Also, the bacteriu may have been modified so as to have a mutant ribosomal protein Si gene (mutant rpsA gene) (WO2013/118544). Also, the bacterium may have been modified so as to have a mutant phoS gene (WO2016/171224). Also, the bacterium may have been modified so that the activity of a RegX3 protein is reduced (WO2018/074578). Also, the bacterium may have been modified so that the activity of a HrrSA system is reduced (WO2018/074579). Also, the bacterium may have been modified so that the activity of the Tat secretion system is increased. These characteristics or modifications may occur alone or in any appropriate combination.

<1-3-1>Introduction of Mutant phoS Gene

The bacterium may have been modified so as to harbor a mutant phoS gene. The expression “to harbor a mutant phoS gene” is also referred to as “to have a mutant phoS gene” or “to have a mutation in a phoS gene”. In addition, the expression “to harbor a mutant phoS gene” is also referred to as “to have a mutant PhoS protein” or “to have a mutation in a PhoS protein”.

Hereinafter, the phoS gene and the PhoS protein will be explained. The phoS gene is a gene encoding a PhoS protein, which is a sensor kinase of the PhoRS system. The PhoRS system is one of two-component regulatory systems, and induces a response against phosphate depletion. The PhoRS system has a sensor kinase PhoS encoded by a phoS gene and a response regulator PhoR encoded by a phoR gene.

A PhoS protein having the “specific mutation” is also referred to as “mutant PhoS protein”, and a gene encoding it is also referred to as “mutant phoS gene”. The mutant phoS gene is, in other words, a phoS gene having the “specific mutation”. Furthermore, a PhoS protein not having the “specific mutation” is also referred to as “wild-type PhoS protein”, and a gene encoding it is also referred to as “wild-type phoS gene”. The wild-type phoS gene is, in other words, a phoS gene not having the “specific mutation”. The term “wild-type” referred to herein is used for convenience to distinguish “wild-type” ones from “mutant” ones, and “wild-type” ones are not limited to those obtained as natural substances, so long as those do not have the “specific mutation”. The “specific mutation” will be described later.

Examples of the wild-type phoS gene include, for example, phoS genes of coryneform bacteria. Specific examples of the phoS genes of coryneform bacteria include, for example, the phoS genes of C. glutamicum YDK010, C. glutamicum ATCC 13032, C. glutamicum ATCC 14067, C. callunae, C. crenatum, and C. efficiens. The nucleotide sequence of the phoS gene of C. glutamicum YDK010 is shown as SEQ ID NO: 28. The amino acid sequences of the wild-type PhoS proteins encoded by these phoS genes are shown as SEQ ID NOS: 29 to 34, respectively.

The wild-type phoS gene may be a variant of any of the wild-type phoS genes exemplified above, so long as it does not have the “specific mutation” and the original function thereof is maintained. Similarly, the wild-type PhoS protein may be a variant of any of the wild-type PhoS proteins exemplified above, so long as it does not have the “specific mutation” and the original function thereof is maintained. That is, the term “wild-type phoS gene” includes not only the wild-type phoS genes exemplified above, but also includes conservative variants thereof that do not have the “specific mutation”. Similarly, the term “wild-type PhoS protein” includes not only the wild-type PhoS proteins exemplified above, but also includes conservative variants thereof that do not have the “specific mutation”. The aforementioned descriptions concerning conservative variants of the LepB protein and the lepB gene can be similarly applied to variants of the wild-type PhoS protein and the wild-type phoS gene. For example, the wild-type phoS gene may also be a gene encoding a protein having any of the aforementioned amino acid sequences, but which includes substitution, deletion, insertion, and/or addition of one or several amino acid residues at one or several positions, so long as it does not have the “specific mutation” and the original function thereof is maintained. Also, for example, the wild-type phoS gene may also be a gene encoding a protein having an amino acid sequence showing an identity of 80% or more, 90% or more, 95% or more, 97% or more, or 99% or more, to the total amino acid sequence of any of the aforementioned amino acid sequences, so long as it does not have the “specific mutation” and the original function thereof is maintained.

Incidentally, the expression “the original function is maintained” used for the wild-type PhoS may mean that a variant of the protein has a function as a PhoS protein (such as a function of a protein consisting of any of the amino acid sequences shown as SEQ ID NOS: 29 to 34). Furthermore, the expression “the original function is maintained” used for the wild-type PhoS protein may also mean that a variant of the protein has a function as a sensor kinase of the PhoRS system. That is, the term “function as a PhoS protein” may specifically refer to a function as a sensor kinase of the PhoRS system. The term “function as a sensor kinase of the PhoRS system” may specifically refer to a function of inducing a response against phosphate depletion in the environment in combination with a response regulator PhoR protein. The term “function as a sensor kinase of the PhoRS system” may more specifically refer to a function of sensing phosphate depletion in the environment to be autophosphorylated, and activating the PhoR protein via transfer of phosphate group.

Whether or not a variant of the PhoS protein has a function as a sensor kinase of the PhoRS system can be confirmed by, for example, introducing a gene encoding the variant into a phoS-gene-deletion strain of a coryneform bacterium, and confirming whether or not responsiveness against phosphate depletion is complemented. Complementation of responsiveness against phosphate depletion can be detected, for example, as improvement of growth under phosphate depletion conditions, or as induction of the expression of genes of which the expression is known to be induced under phosphate depletion conditions (J. Bacteriol., 188, 724-732(2006)). As the phoS-gene-deletion strain of a coryneform bacterium, for example, a phoS-gene-deletion strain of C. glutamicum YDK010 or a phoS-gene-deletion strain of C. glutamicum ATCC 13032 can be used.

It is preferred that a histidine residue that is autophosphorylated is conserved in the wild-type PhoS protein. That is, it is preferred that a conservative mutation occurs at an amino acid residue other than the histidine residue that is autophosphorylated. The term “histidine residue that is autophosphorylated” refers to a histidine residue at position 276 of the wild-type PhoS protein. Furthermore, it is preferred that, for example, the wild-type PhoS protein has a conservative sequence of the wild-type PhoS proteins exemplified above. That is, it is preferred that a conservative mutation occurs at, for example, an amino acid residue not conserved in the wild-type PhoS proteins exemplified above.

The mutant PhoS protein has the “specific mutation” in the amino acid sequence of such a wild-type PhoS protein as described above.

That is, in other words, the mutant PhoS protein may be identical to any of the wild-type PhoS proteins exemplified above or conservative variants thereof except that the mutant PhoS protein has the “specific mutation”. Specifically, the mutant PhoS protein may be, for example, a protein having any of the amino acid sequences shown in SEQ ID NOS: 29 to 34 except that the mutant PhoS protein has the “specific mutation”. Specifically, the mutant PhoS protein may also be, for example, a protein having any of the amino acid sequences shown in SEQ ID NOS: 29 to 34 but including substitution, deletion, insertion, and/or addition of one or several amino acid residues, except that the mutant PhoS protein has the “specific mutation”. Specifically, the mutant PhoS protein may also be, for example, a protein showing an identity of 80% or more, 90% or more, 95% or more, 97% or more, or 99% or more, to any of the amino acid sequences shown in SEQ ID NOS: 29 to 34 except that the mutant PhoS protein has the “specific mutation”.

Furthermore, in other words, the mutant PhoS protein may be a variant of any of the wild-type PhoS proteins exemplified above having the “specific mutation”, and further including a conservative mutation at a site other than that of the “specific mutation”. Specifically, the mutant PhoS protein may be, for example, a protein having any of the amino acid sequences shown in SEQ ID NOS: 29 to 34 but having the “specific mutation”, and further including substitution, deletion, insertion, and/or addition of one or several amino acid residues at a site other than that of the “specific mutation”.

The mutant phoS gene is not particularly limited so long as it encodes such a mutant PhoS protein as described above.

Hereinafter, the “specific mutation” of the mutant PhoS protein will be explained.

The “specific mutation” is not particularly limited, so long as it is a mutation that changes the amino acid sequence of such a wild-type PhoS protein described above, and that is effective for secretory production a heterologous protein.

It is preferred that the “specific mutation” is a mutation that improves the secretory production amount of a heterologous protein. The expression “to improve the secretory production amount of a heterologous protein” means that a coryneform bacterium modified so as to have a mutant phoS gene (modified strain) is able to produce the heterologous protein by secretory production in an amount larger than that obtainable with a non-modified strain. The “non-modified strain” refers to a control strain not having the “specific mutation” in the phoS gene, i.e. a control strain not having any mutant phoS gene, and it may be, for example, a wild-type strain or a parent strain. Although the degree of increase meant by the expression “to produce a heterologous protein by secretory production in an amount larger than that obtainable with a non-modified strain” is not particularly limited so long as the secretory production amount of the heterologous protein is increased compared with that obtainable with a non-modified strain, the expression may mean that the heterologous protein is produced by secretory production in an amount of, for example, 1.1 times or more, 1.2 times or more, 1.3 times or more, 2 times or more, or 5 times or more, of that obtainable with a non-modified strain, in terms of the accumulation amount in the medium and/or on the cell surface layer. In addition, the expression “to produce a heterologous protein by secretory production in an amount larger than that obtainable with a non-modified strain” may also mean that whereas the heterologous protein cannot be detected when a non-concentrated culture supernatant of a non-modified strain is applied to SDS-PAGE and stained with CBB, the heterologous protein can be detected when a non-concentrated culture supernatant of a modified strain is applied to SDS-PAGE and stained with CBB. Incidentally, the expression “to improve the secretory production amount of a heterologous protein” does not necessarily mean that the secretory production amount of every heterologous protein is improved, and it is sufficient that the secretory production amount of a heterologous protein chosen as the target of secretory production is improved. The expression “to improve the secretory production amount of a heterologous protein” may specifically mean, for example, that the secretory production amount of a heterologous protein described in the Example section is improved.

Whether a certain mutation improves the secretory production amount of a heterologous protein can be confirmed by, for example, preparing a strain modified so as to have a gene encoding the PhoS protein having the certain mutation, quantifying the amount of the heterologous protein produced by secretory production when the strain is cultured in a medium, and comparing it with the amount of the heterologous protein produced by secretory production before the modification (non-modified strain) is cultured in the medium.

Examples of the change of the amino acid sequence include substitution of an amino acid residue. That is, it is preferred that the “specific mutation” is replacing an amino acid residue of the wild-type PhoS protein with another amino acid residue. The amino acid residue substituted by the “specific mutation” may be one residue, or may be a combination of two or more residues. The amino acid residue substituted by the “specific mutation” may be an amino acid residue other than the histidine residue that is autophosphorylated. The amino acid residue substituted by the “specific mutation” may be an amino acid residue in the HisKA domain other than the histidine residue that is autophosphorylated. The term “histidine residue that is autophosphorylated” refers to a histidine residue at position 276 of the wild-type PhoS protein. The term “HisKA domain” refers to a region having amino acid residues at positions 266-330 of the wild-type PhoS protein. The amino acid residue substituted by the “specific mutation” may be a tryptophan residue at position 302 of the wild-type PhoS protein (W302).

In the aforementioned mutation, examples of the amino acid residue after substitution include K(Lys), R(Arg), H(His), A(Ala), V(Val), L(Leu), I(Ile), G(Gly), S(Ser), T(Thr), P(Pro), F(Phe), W(Trp), Y(Tyr), C(Cys), M(Met), D(Asp), E(Glu), N(Asn), and Q(Gln), provided that the amino acid residue after substitution is other than the original one. As the amino acid residue after substitution, for example, one resulting in improvement in the secretory production amount of a heterologous protein can be chosen.

When substitution occurs at W302, examples of the amino acid residue after substitution include amino acid residues other than aromatic amino acid and histidine residues. Specific examples of the “amino acid residues other than aromatic amino acid and histidine residues” include K(Lys), R(Arg), A(Ala), V(Val), L(Leu), I(Ile), G(Gly), S(Ser), T(Thr), P(Pro), C(Cys), M(Met), D(Asp), E(Glu), N(Asn), and Q(Gln). More specific examples of the “amino acid residues other than aromatic amino acid and histidine residues” include K(Lys), A(Ala), V(Val), S(Ser), C(Cys), M(Met), D(Asp), and N(Asn).

Incidentally, the term “specific mutation” used for the phoS gene refers to a mutation on the nucleotide sequence thereof that results in such a “specific mutation” as described above into the encoded PhoS protein.

The “amino acid residue at position X of the wild-type PhoS protein” refers to an amino acid residue corresponding to the amino acid residue at position X in SEQ ID NO: 29. For example, “W302” refers to an amino acid residue corresponding to the tryptophan residue at position 302 in SEQ ID NO: 29. The aforementioned positions of amino acid residues indicate relative positions, and the absolute positions thereof may shift due to deletion, insertion, addition, or the like of an amino acid residue or residues. For example, if one amino acid residue is deleted or inserted at a position on the N-terminal side of position X in the wild-type PhoS protein having the amino acid sequence shown as SEQ ID NO: 29, the amino acid residue originally at position X is relocated at position X-1 or X+1 counted from the N-terminus, however, it is still regarded as the “amino acid residue at position X of the wild-type PhoS protein”. Specifically, for example, “W302” refers to the tryptophan residue at positions 302, 302, 302, 321, 275, and 286, respectively, in the amino acid sequences of wild-type PhoS proteins shown in SEQ ID NOS: 29 to 34. Furthermore, the “histidine residue at position 276 of the wild-type PhoS protein (histidine residue that is autophosphorylated)” refers to the histidine residue at positions 276, 276, 276, 295, 249, and 260, respectively, in the amino acid sequences of wild-type PhoS proteins shown in SEQ ID NOS: 29 to 34. Furthermore, the “region having amino acid residues at positions 266-330 of the wild-type PhoS protein (HisKA domain)” refers to the region having amino acid residues at positions 266-330, 266-330, 266-330, 285-349, 239-303, and 250-314, respectively, in the amino acid sequences of wild-type PhoS proteins shown in SEQ ID NOS: 29 to 34.

Incidentally, while “W302” referred to herein is typically a tryptophan residue, it may also be other than a tryptophan residue. That is, when the wild-type PhoS protein has an amino acid sequence other than the amino acid sequences shown in SEQ ID NOS: 29 to 34, “W302” can be other than a tryptophan residue. Hence, for example, the “mutation replacing W302 with a cysteine residue” includes not only a mutation, when “W302” is a tryptophan residue, for replacing this tryptophan residue with a cysteine residue, but also includes a mutation, when “W302” is K(Lys), R(Arg), H(His), A(Ala), V(Val), L(Leu), I(Ile), G(Gly), S(Ser), T(Thr), P(Pro), F(Phe), Y(Tyr), M(Met), D(Asp), E(Glu), N(Asn), or Q(Gln), for replacing this residue with a cysteine residue. The same can be similarly applied to the other mutations.

Which amino acid residue is the “amino acid residue corresponding to the amino acid residue at position X in SEQ ID NO: 29” in the amino acid sequence of an arbitrary PhoS protein can be determined by alignment between the amino acid sequence of the arbitrary PhoS protein and the amino acid sequence of SEQ ID NO: 29. The alignment can be performed by, for example, using known gene analysis software. Specific examples of such software include DNASIS produced by Hitachi Solutions, GENETYX produced by Genetyx, and so forth (Elizabeth C. Tyler et al., Computers and Biomedical Research, 24 (1) 72-96, 1991; Barton G J et al., Journal of Molecular Biology, 198 (2), 327-37, 1987).

The mutant phoS gene can be obtained by, for example, modifying a wild-type phoS gene so that the encoded PhoS protein has the aforementioned “specific mutation”. The wild-type phoS gene to be modified can be obtained by, for example, cloning from an organism having the wild-type phoS gene, or chemical synthesis. Furthermore, the mutant phoS gene can also be obtained without using a wild-type phoS gene. For example, the mutant phoS gene may be directly obtained by chemical synthesis. The obtained mutant phoS gene may be further modified before use.

Genes can be modified by known methods. For example, an objective mutation can be introduced into a target site of DNA by the site-specific mutagenesis method. Examples of the site-specific mutagenesis method include a method of using PCR (Higuchi, R., 61, in PCR Technology, Erlich, H. A. Eds., Stockton Press (1989); Carter P, Meth. In Enzymol., 154, 382 (1987)), and a method of using a phage (Kramer, W and Frits, H. J., Meth. in Enzymol., 154, 350 (1987); Kunkel, T. A. et al., Meth. in Enzymol., 154, 367 (1987)).

Hereinafter, methods for modifying a coryneform bacterium so as to have a mutant phoS gene will be explained.

A coryneform bacterium can be modified so as to have a mutant phoS gene by introducing the mutant phoS gene into the coryneform bacterium. A coryneform bacterium can be modified so as to have a mutant phoS gene also by introducing a mutation into the phoS gene on the chromosome of the coryneform bacterium. A mutation can be introduced into a gene on a chromosome by natural mutation, mutagenesis treatment, or genetic engineering means.

Methods for introducing a mutant phoS gene into a coryneform bacterium are not particularly limited. It is sufficient that the mutant phoS gene is harbored by the bacterium so that it can be expressed under control of a promoter that functions in a coryneform bacterium. The promoter may be a promoter derived from the host, or may be a heterogenous promoter. The promoter may be the native promoter of the phoS gene, or a promoter derived from another gene. In the bacterium, the mutant phoS gene may be present on a vector that autonomously replicates out of the chromosome, such as plasmid, or may be incorporated into the chromosome. The bacterium may have only one copy of the mutant phoS gene, or two or more copies of the mutant phoS gene. The bacterium may have only one kind of mutant phoS gene, or two or more kinds of mutant phoS genes. The mutant phoS gene can be introduced, for example, in the same manner as that for introduction of a gene in methods for increasing the expression of a gene described below, or for introduction of the genetic construct used as described below.

The bacterium may or may not have the wild-type phoS gene. It is preferred that the bacterium does not have the wild-type phoS gene.

A coryneform bacterium not having the wild-type phoS gene can be obtained by disrupting the wild-type phoS gene on the chromosome. The wild-type phoS gene can be disrupted by known methods. Specifically, the wild-type phoS gene can be disrupted by, for example, deleting a portion or the entire the promoter region and/or the coding region of the wild-type phoS gene.

Furthermore, by replacing the wild-type phoS gene on the chromosome with a mutant phoS gene, a coryneform bacterium modified so that it does not have the wild-type phoS gene and has the mutant phoS gene can be obtained. Examples of methods for performing such gene substitution include, for example, a method of using a linear DNA such as a method called “Red-driven integration” (Datsenko, K. A, and Wanner, B. L., Proc. Natl. Acad. Sci. USA, 97:6640-6645 (2000)), a method of utilizing the Red driven integration in combination with an excision system derived from λ phage (Cho, E. H., Gumport, R I., Gardner, J. F., J. Bacteriol., 184:5200-5203 (2002)) (refer to WO2005/010175), a method of using a plasmid including a temperature sensitive replication origin, a method of using a plasmid capable of conjugative transfer, a method of utilizing a suicide vector not including a replication origin that functions in a host (U.S. Pat. No. 6,303,383, Japanese Patent Laid-open (Kokai) No. 05-007491), and so forth.

The PhoS protein functions, that is, induces a response against phosphate depletion in the environment, in combination with a response regulator PhoR protein. Hence, the bacterium has a phoR gene so that the mutant PhoS protein functions. The phoR gene is a gene encoding a PhoR protein, which is a response regulator of the PhoRS system. The expression “to have a phoR gene” is also referred to as “to have a PhoR protein”. Typically, it is sufficient that the PhoR protein inherently possessed by the bacterium functions in combination with the mutant PhoS protein. Alternatively, the bacterium may be introduced with an appropriate phoR gene, in addition to or instead of the phoR gene inherently possessed by the bacterium. The phoR gene to be introduced is not particularly limited, as long as it encodes a PhoR protein that functions in combination with the mutant PhoS protein.

Examples of the phoR gene include, for example, phoR genes of coryneform bacteria. Specific examples of the phoR genes of coryneform bacteria include, for example, the phoR genes of C. glutamicum YDK010, C. glutamicum ATCC 13032, C. glutamicum ATCC 14067, C. callunae, C. crenatum, and C. efficiens. The nucleotide and amino acid sequences of the phoR gene and protein of C. glutamicum ATCC 13032 are shown as SEQ ID NO: 35 and 36, respectively.

The phoR gene may be a variant of any of the phoR genes exemplified above, so long as the original function thereof is maintained. Similarly, the PhoR protein may be a variant of any of the PhoR proteins exemplified above, so long as the original function thereof is maintained. That is, the term “phoR gene” includes not only the phoR genes exemplified above, but also includes conservative variants thereof. Similarly, the term “PhoR protein” includes not only the PhoR proteins exemplified above, but also includes conservative variants thereof. The aforementioned descriptions concerning conservative variants of the LepB protein and the lepB gene can be similarly applied to variants of the phoR gene and PhoR protein. For example, the phoR gene may be a gene encoding a protein having the aforementioned amino acid sequence, but including substitution, deletion, insertion, and/or addition of one or several amino acid residues at one or several positions, so long as the original function is maintained. Also, for example, the phoR gene may also be a gene encoding a protein having an amino acid sequence having an identity of 80% or more, 90% or more, 95% or more, 97% or more, or 99% or more, to the total amino acid sequence of any of the aforementioned amino acid sequences, so long as the original function is maintained. Incidentally, the expression “the original function is maintained” used for the PhoR protein may mean that a variant of the protein has a function as a PhoR protein (such as a function of a protein consisting of the amino acid sequence shown as SEQ ID NO: 36). Furthermore, the expression “the original function is maintained” used for the PhoR protein may also mean that a variant of the protein has a function as a response regulator of the PhoRS system. That is, the term “function as a PhoR protein” may specifically refer to a function as a response regulator of the PhoRS system. The term “function as a response regulator of the PhoRS system” may specifically refer to a function of inducing a response against phosphate depletion in the environment in combination with a sensor kinase PhoS protein. The term “function as a response regulator of the PhoRS system” may more specifically refer to a function of being activated via transfer of phosphate group from the PhoS protein that sensed phosphate depletion in the environment to be autophosphorylated, and regulating the expression of genes that respond to phosphate depletion in the environment.

Whether or not a variant of the PhoR protein functions as a response regulator of the PhoRS system can be confirmed by, for example, introducing a gene encoding the variant into a phoR-gene-deletion strain of a coryneform bacterium, and confirming whether or not responsiveness against phosphate depletion is complemented. Complementation of responsiveness against phosphate depletion can be detected, for example, as improvement of growth under phosphate depletion conditions, or as induction of the expression of genes of which the expression is known to be induced under phosphate depletion conditions (J. Bacteriol., 188, 724-732(2006)). As the phoR-gene-deletion strain of a coryneform bacterium, for example, a phoR-gene-deletion strain of C. glutamicum YDK010 or a phoR-gene-deletion strain of C. glutamicum ATCC 13032 can be used.

<1-3-2>Reduction in Activity of Cell Surface Layer Protein

The bacterium may be a bacterium in which the activity(s) of cell surface layer protein(s) is/are reduced. Specifically, the bacterium may be a bacterium in which the activity(s) of cell surface layer protein(s) is/are reduced as compared with a non-modified strain. The phrase “the activity of a cell surface layer protein is reduced” may particularly mean that the number of molecules of the cell surface layer protein per cell is reduced. Hereinafter, the cell surface layer proteins and genes encoding them will be explained.

The cell surface layer protein is a protein constituting the surface layer (S layer) of bacteria or archaea. Examples of cell surface layer proteins of coryneform bacteria include PS1 and PS2 (CspB) of C. glutamicum (Japanese Patent Laid-open (Kohyo) No. 6-502548), and SlpA (CspA) of C. stationis (Japanese Patent Laid-open (Kokai) No. 10-108675). It is preferable to reduce the activity of the PS2 protein among these.

The nucleotide sequence of the cspB gene of C. glutamicum ATCC 13869 and the amino acid sequence of the PS2 protein (CspB protein) encoded by the gene are shown in SEQ ID NOS: 37 and 38, respectively.

Furthermore, for example, amino acid sequences of CspB homologues were reported for 28 strains of C .glutamicum (J. Biotechnol., 112, 177-193 (2004)). These 28 strains of C. glutamicum and the GenBank accession numbers of the cspB gene homologues in NCBI database are exemplified below (the GenBank accession numbers are shown in the parentheses).

C. glutamicum ATCC 13058 (AY524990)

C. glutamicum ATCC 13744 (AY524991)

C. glutamicum ATCC 13745 (AY524992)

C. glutamicum ATCC 14017 (AY524993)

C. glutamicum ATCC 14020 (AY525009)

C. glutamicum ATCC 14067 (AY524994)

C. glutamicum ATCC 14068 (AY525010)

C. glutamicum ATCC 14747 (AY525011)

C. glutamicum ATCC 14751 (AY524995)

C. glutamicum ATCC 14752 (AY524996)

C. glutamicum ATCC 14915 (AY524997)

C. glutamicum ATCC 15243 (AY524998)

C. glutamicum ATCC 15354 (AY524999)

C. glutamicum ATCC 17965 (AY525000)

C. glutamicum ATCC 17966 (AY525001)

C. glutamicum ATCC 19223 (AY525002)

C. glutamicum ATCC 19240 (AY525012)

C. glutamicum ATCC 21341 (AY525003)

C. glutamicum ATCC 21645 (AY525004)

C. glutamicum ATCC 31808 (AY525013)

C. glutamicum ATCC 31830 (AY525007)

C. glutamicum ATCC 31832 (AY525008)

C. glutamicum LP-6 (AY525014)

C. glutamicum DSM20137 (AY525015)

C. glutamicum DSM20598 (AY525016)

C. glutamicum DSM46307 (AY525017)

C. glutamicum 22220 (AY525005)

C. glutamicum 22243 (AY525006)

Since the nucleotide sequence of a gene encoding a cell surface layer protein may differ depending on species or strain to which the coryneform bacterium belongs, the gene encoding a cell surface layer protein may be a variant of any of genes encoding the cell surface layer proteins exemplified above, so long as the original function thereof is maintained. Similarly, the cell surface layer protein may be a variant of any of the cell surface layer proteins exemplified above, so long as the original function thereof is maintained. That is, the term “cspB gene” includes not only the cspB genes exemplified above, but also includes conservative variants thereof. Similarly, the term “CspB protein” includes not only the CspB proteins exemplified above, but also includes conservative variants thereof. The aforementioned descriptions concerning conservative variants of the LepB protein and the lepB gene can be similarly applied to variants of the cell surface layer protein and the gene encoding it. For example, the gene encoding the cell surface layer protein may be a gene encoding a protein having the aforementioned amino acid sequence, but including substitution, deletion, insertion, and/or addition of one or several amino acid residues at one or several positions, so long as the original function is maintained. Also, for example, the gene encoding the cell surface layer protein may also be a gene encoding a protein having an amino acid sequence showing an identity of 80% or more, 90% or more, 95% or more, 97% or more, or 99% or more, to the total amino acid sequence of any of the aforementioned amino acid sequences, so long as the original function is maintained. Incidentally, the expression “original function is maintained” used for the cell surface layer protein may mean that the protein has a property that if the activity of the protein is reduced in a coryneform bacterium, the secretory production amount of a heterologous protein is increased compared with that obtainable with a non-modified strain.

The “property that if the activity of the protein is reduced in a coryneform bacterium, the secretory production amount of a heterologous protein is increased compared with that obtainable with a non-modified strain” refers to a property imparting an ability to produce a heterologous protein by secretory production in an amount larger than that obtainable with a non-modified strain to a coryneform bacterium when the activity thereof is reduced in the coryneform bacterium. The “non-modified strain” refers to a control strain of which the activity(s) of cell surface layer protein(s) is/are not reduced, and it may be, for example, a wild-type strain or a parent strain. Although the degree of increase meant by the expression “to produce a heterologous protein by secretory production in an amount larger than that obtainable with a non-modified strain” is not particularly limited so long as the secretory production amount of the heterologous protein is increased compared with that obtainable with a non-modified strain, the expression may mean that the heterologous protein is produced by secretory production in an amount of, for example, 1.1 times or more, 1.2 times or more, 1.3 times or more, or 2 times or more, of that obtainable with a non-modified strain, in terms of the accumulation amount in the medium and/or on the cell surface layer. In addition, the expression “to produce a heterologous protein by secretory production in an amount larger than that obtainable with a non-modified strain” may also mean that whereas the heterologous protein cannot be detected when a non-concentrated culture supernatant of a non-modified strain is applied to SDS-PAGE and stained with CBB, the heterologous protein can be detected when a non-concentrated culture supernatant of a modified strain is applied to SDS-PAGE and stained with CBB.

Whether the activity of the protein is reduced in a coryneform bacterium, the secretory production amount of a heterologous protein is increased compared with that obtainable with a non-modified strain can be confirmed by preparing a strain modified so that the activity of the protein is reduced from a strain belonging to the coryneform bacteria, quantifying the secretory production amount of the heterologous protein observed when the modified strain is cultured in a medium, and comparing the quantified amount with the secretory production amount of the heterologous protein observed before being modified (un-modified strain) is cultured in the medium.

The expression “activity of a cell surface layer protein is reduced” includes when a coryneform bacterium has been modified so that the activity of a cell surface layer protein is reduced and a case where the activity of a cell surface layer protein is inherently reduced in a coryneform bacterium. The “case where activity of a cell surface layer protein is inherently reduced in a coryneform bacterium” includes when a coryneform bacterium is inherently deficient in a cell surface layer protein. That is, examples of a coryneform bacterium in which the activity of a cell surface layer protein is reduced include a coryneform bacterium that is inherently deficient in a cell surface layer protein. Examples of the “case where a coryneform bacterium is inherently deficient in a cell surface layer protein” include a case where a coryneform bacterium is inherently deficient in the gene encoding a cell surface layer protein. The expression “a coryneform bacterium is inherently deficient in a cell surface layer protein” may mean that a coryneform bacterium is inherently deficient in one or more proteins selected from cell surface layer protein(s) found in other strain(s) of the species to which the coryneform bacterium belongs. For example, “C. glutamicum is inherently deficient in a cell surface layer protein” may mean that a C. glutamicum strain is inherently deficient in one or more proteins selected from cell surface layer protein(s) found in other C. glutamicum strain(s), i.e. for example, deficient in PS1 and/or PS2 (CspB). Examples of the coryneform bacterium that is inherently deficient in a cell surface layer protein include C. glutamicum ATCC 13032, which is inherently deficient in the cspB gene.

<1-3-3>Protein Secretion System

The bacterium has the Tat system (Tat secretion system), which is a protein secretion system. The bacterium may inherently the Tat secretion system or may be modified so as to have the Tat secretion system. The bacterium may be modified so that the activity of the Tat secretion system is increased. Specifically, the bacterium may be modified so that the activity of the Tat secretion system is increased as compared with a non-modified strain. The activity of the Tat secretion system can be increased by, for example, increasing the expression of one or more genes encoding the Tat secretion system. That is, more specifically, the bacterium may be modified so that the expression of one or more genes encoding the Tat secretion system is increased. Methods for increasing the expression of genes encoding the Tat secretion system are described in Japanese Patent No. 4730302. By modifying a coryneform bacterium so that the activity of the Tat secretion system is increased, it is expected that an ability of the bacterium to produce a heterologous protein, specifically a heterologous protein in a completely-cleaved form, by secretory production when using the TorA signal peptide can be improved, that is, secretory production of a heterologous protein, specifically a heterologous protein in a completely-cleaved form, by the bacterium using the TorA signal peptide can be increased.

Examples of the genes encoding the Tat secretion system include tatA, tatB, tatC, and tatE genes.

Specific examples of the genes encoding the Tat secretion system include tatA, tatB, and tatC genes of C. glutamicum. The tatA, tatB, and tatC genes of C. glutamicum ATCC 13032 correspond to the complementary sequence of positions 1571065-1571382, the sequence of positions 1167110-1167580, and the complementary sequence of positions 1569929-1570873 in the genome sequence registered as GenBank accession NC_003450 (VERSION NC_003450.3 GI:58036263) in NCBI database, respectively. The TatA, TatB, and TatC proteins of C. glutamicum ATCC 13032 have been registered as GenBank accession NP_600707 (version NP_600707.1 GI:19552705, locus_tag=“NCg11434”), GenBank accession NP_600350 (version NP_600350.1 GI:19552348, locus_tag=“NCg11077”), and GenBank accession NP_600706 (version NP_600706.1 GI:19552704, locus_tag=“NCg11433”), respectively. The nucleotide sequences of the tatA, tatB, and tatC genes of C. glutamicum ATCC 13032 and the amino acid sequences of the TatA, TatB, and TatC proteins of the same are shown as SEQ ID NOS: 39 to 44.

Specific examples of the genes encoding the Tat secretion system also include tatA, tatB, tatC, and tatE genes of E. coli. The tatA, tatB, tatC, and tatE genes of E. coli K-12 MG1655 correspond to the sequence of positions 4019968-4020237, the sequence of positions 4020241-4020756, the sequence of positions 4020759-4021535, and the sequence of positions 658170-658373 in the genome sequence registered as GenBank accession NC_000913(VERSION NC_000913.2 GI:49175990) in NCBI database, respectively. The TatA, TatB, TatC, and TatE proteins of E. coli K-12 MG1655 have been registered as GenBank accession NP_418280 (version NP_418280.4 GI:90111653, locus_tag=“b3836”), GenBank accession YP_026270 (version YP_026270.1 GI:49176428, locus_tag=“b3838”), GenBank accession NP_418282 (version NP_418282.1 GI:16131687, locus_tag=“b3839”), and GenBank accession NP_415160 (version NP_415160.1 GI:16128610, locus_tag=“b0627”) , respectively.

The gene encoding the Tat secretion system may be a variant of any of the genes encoding the Tat-secretion-system exemplified above, so long as the original function thereof is maintained. Similarly, the Tat-secretion-system may be a variant of any of the Tat-secretion-systems exemplified above, so long as the original function thereof is maintained. That is, the terms “tatA gene”, “tatB gene”, “tatC gene”, and “tatE gene” include not only the tatA, tatB, tatC, and tatE genes exemplified above, respectively, but also includes conservative variants thereof. Similarly, the terms “TatA protein”, “TatB protein”, “TatC protein”, and “TatE protein” include not only the TatA, TatB, TatC, and TatE proteins exemplified above, respectively, but also includes conservative variants thereof. The aforementioned descriptions concerning conservative variants of the LepB protein and the lepB gene can be similarly applied to variants of the Tat-secretion-system and the gene encoding it. For example, the gene encoding the Tat-secretion-system may have any of the aforementioned amino acid sequences, but include substitution, deletion, insertion, and/or addition of one or several amino acid residues at one or several positions, so long as the original function is maintained. Also, for example, the gene encoding the Tat-secretion-system may also be a gene encoding a protein having an amino acid sequence showing an identity of 80% or more, 90% or more, 95% or more, 97% or more, or 99% or more, to the total amino acid sequence of any of the aforementioned amino acid sequences, so long as the original function is maintained. Incidentally, the expression “original function is maintained” in reference to the Tat-secretion-system may mean that the system has a function of secreting a protein fused with a Tat-dependent signal peptide, such as the TorA signal peptide, at the N-terminus out of the cell.

An increase in the activity of the Tat secretion system can be confirmed by, for example, confirming an increase in the secretory production amount of a protein fused with a Tat-dependent signal peptide, such as the TorA signal peptide, at the N-terminus. The secretory production amount of the protein fused with a Tat-dependent signal peptide at the N-terminus may be increased to, for example, 1.5 times or more, 2 times or more, or 3 times or more, of that of a non-modified strain.

<1-4>Methods for Increasing Activity of Protein

Hereinafter, the methods for increasing the activity of a protein such as the LepB protein, including the methods for increasing the expression of a gene, will be explained.

The expression “the activity of a protein is increased” means that the activity of the protein is increased as compared with a non-modified strain. Specifically, the expression “the activity of a protein is increased” means that the activity of the protein per cell is increased as compared with that of a non-modified strain. The term “non-modified strain” refers to a control strain that has not been modified so that the activity of an objective protein is increased. Examples of the non-modified strain include a wild-type strain and parent strain. Specific examples of the non-modified strain include the respective type strains of the species of bacteria. Specific examples of the non-modified strain also include strains exemplified above in relation to the description of coryneform bacteria. That is, in an embodiment, the activity of a protein may be increased as compared with a type strain, i.e. the type strain of the species to which the chosen bacterium belongs. In another embodiment, the activity of a protein may also be increased as compared with C. glutamicum ATCC 13869. In another embodiment, the activity of a protein may also be increased as compared with C. glutamicum ATCC 13032. In another embodiment, the activity of a protein may also be increased as compared with C. glutamicum AJ12036 (FERM BP-734). In another embodiment, the activity of a protein may also be increased as compared with C. glutamicum YDK010. The state that “the activity of a protein is increased” may also be expressed as “the activity of a protein is enhanced”. More specifically, the expression “the activity of a protein is increased” may mean that the number of molecules of the protein per cell is increased, and/or the function of each molecule of the protein is increased as compared with those of a non-modified strain. That is, the term “activity” in the expression “the activity of a protein is increased” is not limited to the catalytic activity of the protein, but may also mean the transcription amount of a gene (i.e. the amount of mRNA) encoding the protein, or the translation amount of the gene (i.e. the amount of the protein). The term “the number of molecules of a protein per cell” may mean an average value of the number of molecules of the protein per cell. Furthermore, the expression that “the activity of a protein is increased” can mean that the activity of an objective protein is increased in a strain inherently having the activity of the objective protein, and also that the activity of an objective protein is imparted to a strain not inherently having the activity of the objective protein. Furthermore, so long as the activity of the protein is eventually increased, the activity of an objective protein inherently contained in a host may be attenuated and/or eliminated, and then an appropriate type of the objective protein may be imparted to the host.

The degree of the increase in the activity of a protein is not particularly limited, so long as the activity of the protein is increased as compared with a non-modified strain. The activity of the protein may be increased to, for example, 1.5 times or more, 2 times or more, or 3 times or more of that of a non-modified strain. Furthermore, when the non-modified strain does not have the activity of the objective protein, it is sufficient that the protein is produced as a result of introduction of the gene encoding the protein, and for example, the protein may be produced to such an extent that the activity thereof can be measured.

The modification for increasing the activity of a protein can be attained by, for example, increasing the expression of a gene encoding the protein. The expression “the expression of a gene is increased” means that the expression of the gene is increased as compared with a non-modified strain such as a wild-type strain and parent strain. Specifically, the expression “the expression of a gene is increased” means that the expression amount of the gene per cell is increased as compared with that of a non-modified strain. The term “the expression amount of a gene per cell” may mean an average value of the expression amount of the gene per cell. More specifically, the expression “the expression of a gene is increased” may mean that the transcription amount of the gene (i.e. the amount of mRNA) is increased, and/or the translation amount of the gene (i.e. the amount of the protein expressed from the gene) is increased. The state that “the expression of a gene is increased” may also be referred to as “the expression of a gene is enhanced”. The expression of a gene may be increased to, for example, 1.5 times or more, 2 times or more, or 3 times or more of that of a non-modified strain. Furthermore, the expression “the expression of a gene is increased” can mean not only that the expression amount of an objective gene is increased in a strain that inherently expresses the objective gene, but also that the gene is introduced into a strain that does not inherently express the objective gene, and expressed therein. That is, the phrase “the expression of a gene is increased” may also mean, for example, that an objective gene is introduced into a strain that does not possess the gene, and is expressed therein.

The expression of a gene can be increased by, for example, increasing the copy number of the gene.

The copy number of a gene can be increased by introducing the gene into the chromosome of a host. A gene can be introduced into a chromosome by, for example, using homologous recombination (Miller, J. H., Experiments in Molecular Genetics, 1972, Cold Spring Harbor Laboratory). Examples of the gene transfer method utilizing homologous recombination include, for example, a method of using a linear DNA such as Red-driven integration (Datsenko, K. A., and Wanner, B. L., Proc. Natl. Acad. Sci. USA, 97:6640-6645 (2000)), a method of using a plasmid containing a temperature sensitive replication origin, a method of using a plasmid capable of conjugative transfer, a method of using a suicide vector not having a replication origin that functions in a host, and a transduction method using a phage. Specifically, a host can be transformed with a recombinant DNA containing an objective gene, so that homologous recombination occurs at a target region on the chromosome of the host, to thereby introduce the objective gene into the chromosome of the host. The structure of the recombinant DNA to be used for homologous recombination is not particularly limited as long as it causes homologous recombination in a desired manner. For example, a host can be transformed with a linear DNA containing an objective gene and further containing nucleotide sequences homologous to upstream and downstream of a target region on the chromosome at the respective ends of the objective gene, so that homologous recombination occurs at each of upstream and downstream of the target region, to thereby replace the target region with the objective gene. The recombinant DNA to be used for homologous recombination may contain a marker gene for selection of transformants. Only one copy, or two or more copies of a gene may be introduced. For example, by performing homologous recombination using a nucleotide sequence which is present in multiple copies on a chromosome as a target, multiple copies of a gene can be introduced into the chromosome. Examples of such a sequence which is present in multiple copies on a chromosome include repetitive DNAs, and inverted repeats located at both ends of a transposon. Alternatively, homologous recombination may be performed by using an appropriate sequence on a chromosome such as a gene unnecessary for production of an objective substance as a target. Furthermore, a gene can also be randomly introduced into a chromosome by using a transposon or Mini-Mu (Japanese Patent Laid-open (Kokai) No. 2-109985, U.S. Pat. No. 5,882,888, EP805867B1). Such methods for modifying a chromosome using homologous recombination can be used for any modification on a chromosome, such as a modification of an expression control sequence, as well as for introduction of an objective gene.

Introduction of an objective gene into a chromosome can be confirmed by Southern hybridization using a probe having a sequence complementary to the whole gene or a part thereof, PCR using primers prepared on the basis of the sequence of the gene, or the like.

Furthermore, the copy number of a gene can also be increased by introducing a vector containing the gene into a host. For example, the copy number of a target gene can be increased by ligating a DNA fragment containing the target gene with a vector that functions in a host to construct an expression vector of the gene, and transforming the host with the expression vector. The DNA fragment containing the target gene can be obtained by, for example, PCR using the genomic DNA of a microorganism having the target gene as the template. As the vector, a vector autonomously replicable in the cell of the host can be used. The vector can be a multi-copy vector. Furthermore, the vector can have a marker such as an antibiotic resistance gene for selection of transformant. Furthermore, the vector may have a promoter and/or terminator for expressing the introduced gene. The vector may be, for example, a vector derived from a bacterial plasmid, a vector derived from a yeast plasmid, a vector derived from a bacteriophage, cosmid, phagemid, or the like. Specific examples of vector autonomously replicable in coryneform bacteria include, for example, pHM1519 (Agric. Biol. Chem., 48, 2901-2903 (1984)); pAM330 (Agric. Biol. Chem., 48, 2901-2903 (1984)); plasmids obtained by improving these and having a drug resistance gene; plasmid pCRY30 (Japanese Patent Laid-open (Kokai) No. 3-210184); plasmids pCRY21, pCRY2KE, pCRY2KX, pCRY31, pCRY3KE, and pCRY3KX (Japanese Patent Laid-open (Kokai) No. 2-72876 and U.S. Pat. No. 5,185,262); plasmids pCRY2 and pCRY3 (Japanese Patent Laid-open (Kokai) No. 1-191686); pAJ655, pAJ611, and pAJ1844 (Japanese Patent Laid-open (Kokai) No. 58-192900); pCG1 (Japanese Patent Laid-open (Kokai) No. 57-134500); pCG2 (Japanese Patent Laid-open (Kokai) No. 58-35197); pCG4 and pCG11 (Japanese Patent Laid-open (Kokai) No. 57-183799); pVK7 (Japanese Patent Laid-open (Kokai) No. 10-215883); pVK9 (US2006-0141588); pVC7 (Japanese Patent Laid-open (Kokai) No. 9-070291); pVS7 (WO2013/069634).

When a gene is introduced, it is sufficient that the gene is expressible by the host. Specifically, it is sufficient that the gene is harbored by a host so that it is expressed under control by a promoter that functions in the host. The promoter is not particularly limited so long as it functions in the host. The phrase “promoter that functions in a host” refers to a promoter that shows a promoter activity in the host. The promoter may be a promoter derived from the host, or a heterogenous promoter. The promoter may be the native promoter of the gene to be introduced, or a promoter of another gene. As the promoter, for example, such a stronger promoter as mentioned later may also be used.

A terminator for termination of gene transcription may be located downstream of the gene. The terminator is not particularly limited so long as it functions in the host. The terminator may be a terminator derived from the host, or a heterogenous terminator. The terminator may be the native terminator of the gene to be introduced, or a terminator of another gene.

Vectors, promoters, and terminators available in various microorganisms are disclosed in detail in “Fundamental Microbiology Vol. 8, Genetic Engineering, KYORITSU SHUPPAN CO., LTD, 1987”, and those can be used.

Furthermore, when two or more of genes are introduced, it is sufficient that the genes each are expressible by the host. For example, all the genes may be carried by a single expression vector or a chromosome. Furthermore, the genes may be separately carried by two or more expression vectors, or separately carried by a single or two or more expression vectors and a chromosome. An operon having two or more genes may also be introduced.

The gene to be introduced is not particularly limited so long as it encodes a protein that functions in the host. The gene to be introduced may be a gene derived from or native to the host, or may be a heterogenous gene. The gene to be introduced can be obtained by, for example, PCR using primers designed on the basis of the nucleotide sequence of the gene, and using the genomic DNA of an organism having the gene, a plasmid carrying the gene, or the like as a template. The gene to be introduced may also be totally synthesized, for example, on the basis of the nucleotide sequence of the gene (Gene, 60(1), 115-127 (1987)). The obtained gene can be used as it is, or after being modified as required. That is, a gene can be modified to obtain a variant thereof. A gene can be modified by a known technique. For example, an objective mutation can be introduced into an objective site of DNA by the site-specific mutation method. That is, the coding region of a gene can be modified by the site-specific mutation method so that a specific site of the encoded protein include substitution, deletion, insertion, and/or addition of amino acid residues. Examples of the site-specific mutation method include the method utilizing PCR (Higuchi, R., 61, in PCR Technology, Erlich, H. A. Eds., Stockton Press (1989); Carter, P, Meth. in Enzymol., 154, 382 (1987)), and the method utilizing phage (Kramer, W and Frits, H. J., Meth. in Enzymol., 154, 350 (1987); Kunkel, T. A. et al., Meth. in Enzymol., 154, 367 (1987)). Alternatively, a variant of a gene may be totally synthesized.

Incidentally, when a protein functions as a complex made up of a plurality of subunits, some or all of the subunits may be modified, so long as the activity of the protein is eventually increased. That is, for example, when the activity of a protein is increased by increasing the expression of a gene, the expression of some or all of the genes that encode the subunits may be enhanced. It is usually preferable to enhance the expression of all of genes encoding the subunits. Furthermore, the subunits constituting the complex may be derived from or native to a single kind of organism or two or more kinds of organisms, so long as the complex has a function of the objective protein. That is, for example, genes of the same organism encoding a plurality of subunits may be introduced into a host, or genes of different organisms encoding a plurality of subunits may be introduced into a host.

Furthermore, the expression of a gene can be increased by improving the transcription efficiency of the gene. In addition, the expression of a gene can also be increased by improving the translation efficiency of the gene. The transcription efficiency of the gene and the translation efficiency of the gene can be improved by, for example, modifying an expression control sequence of the gene. The phrase “expression control sequence” collectively refers to sites that affect the expression of a gene. Examples of the expression control sequence include, for example, promoter, Shine-Dalgarno (SD) sequence (also referred to as ribosome binding site (RBS)), and spacer region between RBS and the start codon. Expression control sequences can be identified by using a promoter search vector or gene analysis software such as GENETYX. These expression control sequences can be modified by, for example, a method of using a temperature sensitive vector, or the Red driven integration method (WO2005/010175).

The transcription efficiency of a gene can be improved by, for example, replacing the promoter of the gene on a chromosome with a stronger promoter. The term “stronger promoter” refers to a promoter providing improved transcription of a gene compared with an the inherent wild-type promoter of the gene. Examples of stronger promoters usable in coryneform bacteria include, for example, the artificially modified P54-6 promoter (Appl. Microbiol. Biotechnol., 53, 674-679 (2000)), pta, aceA, aceB, adh, and amyE promoters inducible in coryneform bacteria with acetic acid, ethanol, pyruvic acid, or the like, cspB, SOD, and tuf (EF-Tu) promoters, which are strong promoters capable of providing a large expression amount in coryneform bacteria (Journal of Biotechnology, 104 (2003) 311-323; Appl. Environ. Microbiol., 2005 December; 71 (12):8587-96), as well as lac promoter, tac promoter, and trc promoter. Furthermore, as the stronger promoter, a highly-active type of an existing promoter may also be obtained by using various reporter genes. For example, by making the -35 and -10 regions in a promoter region closer to the consensus sequence, the activity of the promoter can be enhanced (WO00/18935). Examples of highly active-type promoter include various tac-like promoters (Katashkina J I et al., Russian Federation Patent Application No. 2006134574). Methods for evaluating the strength of promoters and examples of strong promoters are described in the paper of Goldstein et al. (Prokaryotic Promoters in Biotechnology, Biotechnol. Annu. Rev., 1, 105-128 (1995)), and so forth.

The translation efficiency of a gene can be improved by, for example, replacing the Shine-Dalgarno (SD) sequence (also referred to as ribosome binding site (RBS)) for the gene on a chromosome with a stronger SD sequence. The “stronger SD sequence” means a SD sequence that provides an improved translation of mRNA compared with the inherent wild-type SD sequence of the gene. Examples of stronger SD sequences include, for example, RBS of the gene 10 derived from phage T7 (Olins P. O. et al, Gene, 1988, 73, 227-235). Furthermore, it is known that substitution, insertion, or deletion of several nucleotides in a spacer region between RBS and the start codon, especially in a sequence immediately upstream of the start codon (5′-UTR), significantly affects the stability and translation efficiency of mRNA, and hence, the translation efficiency of a gene can also be improved by modifying them.

The translation efficiency of a gene can also be improved by, for example, modifying codons. For example, the translation efficiency of the gene can be improved by replacing a rare codon present in the gene with a synonymous codon more frequently used. That is, the gene to be introduced may be modified, for example, so as to have optimal codons according to the codon usage frequency of a host to be used. Codons can be replaced by, for example, the site-specific mutation method. Alternatively, a gene fragment in which objective codons are replaced may be totally synthesized. Frequencies of codons in various organisms are disclosed in the “Codon Usage Database” (kazusa.or.jp/codon; Nakamura, Y. et al, Nucl. Acids Res., 28, 292 (2000)).

Furthermore, the expression of a gene can also be increased by amplifying a regulator that increases the expression of the gene, or deleting or attenuating a regulator that reduces the expression of the gene.

Such methods for increasing the gene expression as mentioned above may be used independently or in any appropriate combination.

Furthermore, the modification that increases the activity of a protein can also be attained by, for example, enhancing the specific activity of the protein. A protein showing an enhanced specific activity can be obtained by, for example, searching various organisms. Furthermore, a highly-active type of an existing protein may also be obtained by introducing a mutation into the existing protein. The mutation to be introduced may be, for example, substitution, deletion, insertion, and/or addition of one or several amino acid residues at one or several position of the protein. The mutation can be introduced by, for example, such a site-specific mutation method as mentioned above. The mutation may also be introduced by, for example, a mutagenesis treatment. Examples of the mutagenesis treatment include irradiation of X-ray, irradiation of ultraviolet, and a treatment with a mutation agent such as N-methyl-N′-nitro-N-nitrosoguanidine (MNNG), ethyl methanesulfonate (EMS), and methyl methanesulfonate (MMS). Furthermore, a random mutation may be induced by directly treating DNA in vitro with hydroxylamine. Enhancement of the specific activity may be independently used, or may be used in any appropriate combination with such methods for enhancing gene expression as mentioned above.

The method for the transformation is not particularly limited, and conventionally known methods can be used. There can be used, for example, a method of treating recipient cells with calcium chloride so as to increase the permeability thereof for DNA, which has been reported for the Escherichia coli K-12 strain (Mandel, M. and Higa, A., J. Mol. Biol., 1970, 53, 159-162), and a method of preparing competent cells from cells which are in the growth phase, followed by transformation with DNA, which has been reported for Bacillus subtilis (Duncan, C. H., Wilson, G. A. and Young, F. E., Gene, 1977, 1:153-167). Alternatively, there can also be used a method of making DNA-recipient cells into protoplasts or spheroplasts, which can easily take up recombinant DNA, followed by introducing a recombinant DNA into the DNA-recipient cells, which is known to be applicable to Bacillus subtilis, actinomycetes, and yeasts (Chang, S. and Choen, S. N., 1979, Mol. Gen. Genet., 168:111-115; Bibb, M. J., Ward, J. M. and Hopwood, O. A., 1978, Nature, 274:398-400; Hinnen, A., Hicks, J. B. and Fink, G. R., 1978, Proc. Natl. Acad. Sci. USA, 75:1929-1933). Furthermore, the electric pulse method reported for coryneform bacteria (Japanese Patent Laid-open (Kokai) No. 2-207791) can also be used.

An increase in the activity of a protein can be confirmed by measuring the activity of the protein.

An increase in the activity of a protein can also be confirmed by confirming an increase in the expression of a gene encoding the protein. An increase in the expression of a gene can be confirmed by confirming an increase in the transcription amount of the gene, or by confirming an increase in the amount of a protein expressed from the gene.

An increase of the transcription amount of a gene can be confirmed by comparing the amount of mRNA transcribed from the gene with that of a non-modified strain such as a wild-type strain or parent strain. Examples of the method for evaluating the amount of mRNA include Northern hybridization, RT-PCR, microarray, RNA-seq, and so forth (Sambrook, J., et al., Molecular Cloning A Laboratory Manual/Third Edition, Cold Spring Harbor Laboratory Press, Cold Spring Harbor (USA), 2001). The amount of mRNA (such as the number of molecules of the mRNA per cell) may be increased to, for example, 1.5 times or more, 2 times or more, or 3 times or more of that of a non-modified strain.

An increase in the amount of a protein can be confirmed by Western blotting using antibodies (Sambrook, J., et al., Molecular Cloning A Laboratory Manual/Third Edition, Cold Spring Harbor Laboratory Press, Cold Spring Harbor (USA), 2001). The amount of the protein (such as the number of molecules of the protein per cell) may be increased to, for example, 1.5 times or more, 2 times or more, or 3 times or more of that of a non-modified strain.

The aforementioned methods for increasing the activity of a protein can be used for enhancement of the activities of any proteins and enhancement of the expression of any genes.

<1-5>Method for Reducing Activity of Protein

Hereinafter, methods for reducing the activity of a protein will be explained. The methods for reducing the activity of a protein described below can also be utilized for disruption of the wild-type PhoS protein.

The expression “the activity of a protein is reduced” means that the activity of the protein is reduced as compared with a non-modified strain. Specifically, the expression “the activity of a protein is reduced” means that the activity of the protein per cell is reduced as compared with that of a non-modified strain. The term “non-modified strain” used herein refers to a control strain that has not been modified so that the activity of an objective protein is reduced. Examples of the non-modified strain include a wild-type strain and parent strain. Specific examples of the non-modified strain include the respective type strains of the species of bacteria. Specific examples of the non-modified strain also include strains exemplified above in relation to the description of coryneform bacteria. That is, in an embodiment, the activity of a protein may be reduced as compared with a similar strain, i.e. the same or similar strain of the species to which the bacterium belongs. In another embodiment, the activity of a protein may also be reduced as compared with C. glutamicum ATCC 13032. In another embodiment, the activity of a protein may also be reduced as compared with C. glutamicum ATCC 13869. In another embodiment, the activity of a protein may also be reduced as compared with C. glutamicum AJ12036 (FERM BP-734). In another embodiment, the activity of a protein may also be reduced as compared with C. glutamicum YDK010. The state that “the activity of a protein is reduced” also includes a state that the activity of the protein has completely disappeared. More specifically, the expression “the activity of a protein is reduced” may mean that the number of molecules of the protein per cell is reduced, and/or the function of each molecule of the protein is reduced as compared with those of a non-modified strain. That is, the term “activity” in the expression “the activity of a protein is reduced” is not limited to the catalytic activity of the protein, but may also mean the transcription amount of a gene (i.e. the amount of mRNA) encoding the protein or the translation amount of the gene (i.e. the amount of the protein). The phrase “the number of molecules of a protein per cell” may mean an average value per cell of the number of molecules of the protein. The state that “the number of molecules of the protein per cell is reduced” also includes a state that the protein does not exist at all. The state that “the function of each molecule of the protein is reduced” also includes a state that the function of each protein molecule has completely disappeared. The degree of the reduction in the activity of a protein is not particularly limited, so long as the activity is reduced as compared with that of a non-modified strain. The activity of a protein may be reduced to, for example, 50% or less, 20% or less, 10% or less, 5% or less, or 0% of that of a non-modified strain.

The modification for reducing the activity of a protein can be attained by, for example, reducing the expression of a gene encoding the protein. The expression “the expression of a gene is reduced” means that the expression of the gene is reduced as compared with a non-modified strain. Specifically, the expression “the expression of a gene is reduced” means that the expression amount of the gene per cell is reduced as compared with that of a non-modified strain. The phrase “the expression amount of a gene per cell” may mean an average value per cell of the expression amount of the gene. More specifically, the expression “the expression of a gene is reduced” may mean that the transcription amount of the gene (i.e. the amount of mRNA) is reduced, and/or the translation amount of the gene (i.e. the amount of the protein expressed from the gene) is reduced. The phrase that “the expression of a gene is reduced” also includes when the gene is not expressed at all. The phrase that “the expression of a gene is reduced” can also mean “the expression of a gene is attenuated”. The expression of a gene may be reduced to, for example, 50% or less, 20% or less, 10% or less, 5% or less, or 0% of that of a non-modified strain.

The reduction in gene expression may be due to, for example, a reduction in the transcription efficiency, a reduction in the translation efficiency, or a combination of them. The expression of a gene can be reduced by modifying an expression control sequence of the gene. The term “expression control sequence” collectively refers to sites that affect the expression of a gene, such as a promoter, Shine-Dalgarno (SD) sequence (also referred to as ribosome-binding site (RBS)), and spacer region between RBS and the start codon. Expression control sequences can be identified by, for example, using a promoter search vector or gene analysis software such as GENETYX. When an expression control sequence is modified, one or more nucleotides, two or more nucleotides, three or more nucleotides, of the expression control sequence are modified. The transcription efficiency of a gene can be reduced by, for example, replacing the promoter of the gene on a chromosome with a weaker promoter. The term “weaker promoter” means a promoter providing an attenuated transcription of a gene compared with an inherently existing wild-type promoter of the gene. Examples of weaker promoters include, for example, inducible promoters. That is, an inducible promoter may function as a weaker promoter under a non-induced condition, such as in the absence of the corresponding inducer. Furthermore, portion of or the entire expression control sequence may be deleted. The expression of a gene can also be reduced by, for example, manipulating a factor responsible for expression control. Examples of the factor responsible for expression control include low molecules responsible for transcription or translation control (inducers, inhibitors, etc.), proteins responsible for transcription or translation control (transcription factors etc.), nucleic acids responsible for transcription or translation control (siRNA etc.), and so forth. Furthermore, the expression of a gene can also be reduced by, for example, introducing a mutation that reduces the expression of the gene into the coding region of the gene. For example, the expression of a gene can be reduced by replacing a codon in the coding region of the gene with a synonymous codon used less frequently in a host. Furthermore, for example, the gene expression may be reduced due to disruption of a gene as described later.

The modification for reducing the activity of a protein can also be attained by, for example, disrupting a gene encoding the protein. The expression “a gene is disrupted” means that a gene is modified so that a protein that can normally function is not produced. The phrase “a protein that normally functions is not produced” includes when the protein is not produced at all from the gene, and when the protein of which the function (such as activity or property) per molecule is reduced or eliminated is produced from the gene.

Disruption of a gene can be attained by, for example, deleting the gene on a chromosome. The term “deletion of a gene” refers to deletion of a partial or entire region of the coding region of the gene. Furthermore, the entire gene including sequences upstream and downstream from the coding region of the gene on a chromosome may be deleted. The sequences upstream and downstream from the coding region of the gene may include, for example, an expression control sequence of the gene. The region to be deleted may be any region such as an N-terminal region (region encoding an N-terminal region of a protein), an internal region, or a C-terminal region (region encoding a C-terminal region of a protein), so long as the activity of the protein can be reduced. Deletion of a longer region can usually more surely inactivate the gene. The region to be deleted may be, for example, a region having a length of 10% or more, 20% or more, 30% or more, 40% or more, 50% or more, 60% or more, 70% or more, 80% or more, 90% or more, or 95% or more of the total length of the coding region of the gene. Furthermore, it is preferred that reading frames of the sequences upstream and downstream from the region to be deleted are not the same. Inconsistency of reading frames may cause a frameshift downstream of the region to be deleted.

Disruption of a gene can also be attained by, for example, introducing a mutation for an amino acid substitution (missense mutation), a stop codon (nonsense mutation), addition or deletion of one or two nucleotide residues (frame shift mutation), or the like into the coding region of the gene on a chromosome (Journal of Biological Chemistry, 272:8611-8617 (1997); Proceedings of the National Academy of Sciences, USA, 95 5511-5515 (1998); Journal of Biological Chemistry, 26 116, 20833-20839 (1991)).

Disruption of a gene can also be attained by, for example, inserting another nucleotide sequence into a coding region of the gene on a chromosome. Site of the insertion may be in any region of the gene, and insertion of a longer nucleotide sequence can usually more surely inactivate the gene. It is preferred that reading frames of the sequences upstream and downstream from the insertion site are not the same. Inconsistency of reading frames may cause a frameshift downstream of the region to be deleted. The other nucleotide sequence is not particularly limited so long as a sequence that reduces or eliminates the activity of the encoded protein is chosen, and examples thereof include, for example, a marker gene such as antibiotic resistance genes, and a gene useful for production of an objective substance.

Particularly, disruption of a gene may be carried out so that the amino acid sequence of the encoded protein is deleted. In other words, the modification for reducing the activity of a protein can be attained by, for example, deleting the amino acid sequence of the protein, specifically, modifying a gene so as to encode a protein of which the amino acid sequence is deleted. The term “deletion of the amino acid sequence of a protein” refers to deletion of a partial or entire region of the amino acid sequence of the protein. In addition, the term “deletion of the amino acid sequence of a protein” means that the original amino acid sequence is not present in the protein, and also includes when the original amino acid sequence is changed to another amino acid sequence. That is, for example, a region that was changed to another amino acid sequence by frameshift may be regarded as a deleted region. When the amino acid sequence of a protein is deleted, the total length of the protein is typically shortened, but there can also be cases where the total length of the protein is not changed or is extended. For example, by deletion of a portion of or the entire coding region of a gene, a region encoded by the deleted region can be deleted in the encoded protein. In addition, for example, by introduction of a stop codon into the coding region of a gene, a region encoded by the downstream region of the introduction site can be deleted in the encoded protein. In addition, for example, by frameshift in the coding region of a gene, a region encoded by the frameshift region can be deleted in the encoded protein. The aforementioned descriptions concerning the position and length of the region to be deleted in deletion of a gene can be similarly applied to the position and length of the region to be deleted in deletion of the amino acid sequence of a protein.

Such modification of a gene on a chromosome as described above can be attained by, for example, preparing a disruption-type gene modified so that it is unable to produce a protein that normally functions, and transforming a host with a recombinant DNA containing the disruption-type gene to cause homologous recombination between the disruption-type gene and the wild-type gene on a chromosome and thereby substitute the disruption-type gene for the wild-type gene on the chromosome. In this procedure, if a marker gene selected according to the characteristics of the host such as auxotrophy is included in the recombinant DNA, the operation becomes easier. Examples of the disruption-type gene include a gene in which a portion or the entire coding region is deleted, gene including a missense mutation, gene including a nonsense mutation, gene including a frame shift mutation, and gene inserted with an insertion sequence such as a transposon or marker gene. The protein encoded by the disruption-type gene has a conformation different from that of the wild-type protein, even if it is produced, and thus the function thereof is reduced or eliminated. The structure of the recombinant DNA to be used for homologous recombination is not particularly limited as long as it causes homologous recombination in a desired manner. For example, a host can be transformed with a linear DNA containing the disruption-type gene and further containing upstream and downstream sequences of the wild-type gene on the chromosome at the respective ends, so that homologous recombination occurs at each of upstream and downstream sides of the wild-type gene, to thereby replace the wild-type gene with the disruption-type gene. Such gene disruption based on gene substitution utilizing homologous recombination has already been established, and there are methods of using a linear DNA such as a method called “Red driven integration” (Datsenko, K. A, and Wanner, B. L., Proc. Natl. Acad. Sci. USA, 97:6640-6645 (2000)), and a method utilizing the Red driven integration in combination with an excision system derived from λ phage (Cho, E. H., Gumport, R I., Gardner, J. F., J. Bacteriol., 184:5200-5203 (2002)) (refer to WO2005/010175), a method of using a plasmid having a temperature sensitive replication origin, a method of using a plasmid capable of conjugative transfer, a method of utilizing a suicide vector not having a replication origin that functions in a host (U.S. Pat. No. 6,303,383, Japanese Patent Laid-open (Kokai) No. 05-007491), and so forth. Such methods for modifying a chromosome using homologous recombination can be used for any modification on a chromosome, such as a modification of an expression control sequence, as well as for disruption of an objective gene.

A modification for reducing activity of a protein can also be attained by, for example, a mutagenesis treatment. Examples of the mutagenesis treatment include irradiation of X-ray or ultraviolet and treatment with a mutation agent such as N-methyl-N′-nitro-N-nitrosoguanidine (MNNG), ethyl methanesulfonate (EMS), and methyl methanesulfonate (MMS).

Such methods for reducing the activity of a protein as mentioned above may be used independently or in an arbitrary combination.

A reduction in the activity of a protein can be confirmed by measuring the activity of the protein.

A reduction in the activity of a protein can also be confirmed by confirming a reduction in the expression of a gene encoding the protein. A reduction in the expression of a gene can be confirmed by confirming a reduction in the transcription amount of the gene or a reduction in the amount of the protein expressed from the gene.

A reduction in the transcription amount of a gene can be confirmed by comparing the amount of mRNA transcribed from the gene with that of a non-modified strain. Examples of the method for evaluating the amount of mRNA include Northern hybridization, RT-PCR, microarray, RNA-Seq, and so forth (Sambrook, J., et al., Molecular Cloning: A Laboratory Manual/Third Edition, Cold Spring Harbor Laboratory Press, Cold Spring Harbor (USA), 2001). The amount of mRNA may be reduced to, for example, 50% or less, 20% or less, 10% or less, 5% or less, or 0% of that of a non-modified strain.

A reduction in the amount of a protein can be confirmed by performing SDS-PAGE and confirming the intensity of the separated protein band. A reduction in the amount of a protein can be confirmed by Western blotting using antibodies (Sambrook, J., et al., Molecular Cloning: A Laboratory Manual/Third Edition, Cold Spring Harbor Laboratory Press, Cold Spring Harbor (USA), 2001). The amount of the protein (such as the number of molecules of the protein per cell) may be reduced to, for example, 50% or less, 20% or less, 10% or less, 5% or less, or 0% of that of a non-modified strain.

Disruption of a gene can be confirmed by determining nucleotide sequence of a part or the whole of the gene, restriction enzyme map, full length, or the like of the gene depending on the means used for the disruption.

The aforementioned methods for reducing the activity of a protein as mentioned above can be applied to reduction in the activities of any proteins and reduction in the expression of any genes.

<1-6>Genetic Construct for Secretory Expression of Heterologous Protein and Introduction of the Same

It is known that a secretory protein is generally translated as a preprotein (also referred to as prepeptide) or a preproprotein (also referred to as prepropeptide), and then becomes a mature protein through processing. Specifically, a secretory protein is generally translated as a preprotein or preproprotein, then a signal peptide as the pre-moiety is cleaved with a protease (generally called signal peptidase), and the secretory protein is thereby converted into a mature protein or proprotein. As for the proprotein, the pro-moiety thereof is further cleaved by a protease, and the proprotein thereby becomes a mature protein. Therefore, a signal peptide is used for the secretory production of a heterologous protein in the method as described herein. A preprotein and a preproprotein of a secretory protein may be collectively referred to as “secretory protein precursor”.

The genetic construct includes, in the direction from 5′ to 3′, a promoter sequence that functions in a coryneform bacterium, a nucleic acid sequence encoding a TorA signal peptide, and a nucleic acid sequence encoding a heterologous protein. The nucleic acid sequence encoding the TorA signal peptide may be ligated downstream from the promoter sequence so that the TorA signal peptide is expressed under the control of the promoter. The nucleic acid sequence encoding the heterologous protein may be ligated downstream from the nucleic acid sequence encoding the TorA signal peptide so that the heterologous protein is expressed as a fusion protein with the signal peptide. This fusion protein is also referred to as “fusion protein of the present invention”. As described above, in the method, a heterologous protein from which the TorA signal peptide has been completely removed, i.e. a heterologous protein in a completely-cleaved form, is produced by secretory production. That is, the heterologous protein eventually obtained does not has the TorA signal peptide. Hence, the expression “a heterologous protein is expressed as a fusion protein with a TorA signal peptide” means that the heterologous protein constitutes a fusion protein with the TorA signal peptide at the time of expression, and it does not mean that the eventually-obtained heterologous protein constitutes a fusion protein with the TorA signal peptide. A nucleic acid sequence may also be read as “gene”. For example, a nucleic acid sequence encoding a heterologous protein is also referred to as “gene encoding a heterologous protein” or “heterologous protein gene”. Examples of the nucleic acid sequence include DNA. The genetic construct may also include a control sequence (operator, SD sequence, terminator, etc.) effective for expression of the fusion protein in a coryneform bacterium at such an appropriate position that it can function.

The promoter is not particularly limited so long as a promoter that functions in the chosen coryneform bacterium. The promoter may be a promoter derived from a coryneform bacterium, such as one derived from the host, or it may be a heterologous promoter. The promoter may be the native promoter of the heterologous protein, or a promoter of another gene. The “promoter that functions in a coryneform bacterium” refers to a promoter that possesses promoter activity in a coryneform bacterium.

Specific examples of the heterologous promoter include, for example, promoters derived from E. coli such as tac promoter, lac promoter, tip promoter, and araBAD promoter. Among these, strong promoters such as tac promoter and inducible promoters such as araBAD promoter are preferred.

Examples of the promoter derived from a coryneform bacterium include, for example, promoters of the genes of the cell surface layer proteins PS1, PS2 (also referred to as CspB), and SlpA (also referred to as CspA), and promoters of various amino acid biosynthesis system genes. Specific examples of the promoters of various amino acid biosynthesis system genes include, for example, promoters of the glutamate dehydrogenase gene of the glutamic acid biosynthesis system, the glutamine synthetase gene of the glutamine synthesis system, the aspartokinase gene of the lysine biosynthesis system, the homoserine dehydrogenase gene of the threonine biosynthesis system, the acetohydroxy acid synthetase gene of the isoleucine and valine biosynthesis system, 2-isopropylmalate synthetase gene of the leucine biosynthesis system, the glutamate kinase gene of the proline and arginine biosynthesis system, the phosphoribosyl-ATP pyrophosphorylase gene of the histidine biosynthesis system, the deoxyarabinoheptulonate phosphate (DAHP) synthetase gene of the aromatic amino acid biosynthesis systems such as those for tryptophan, tyrosine, and phenylalanine, the phosphoribosyl pyrophosphate (PRPP) amidotransferase gene of the nucleic acid biosynthesis systems such as those for inosinic acid and guanylic acid, the inosinic acid dehydrogenase gene, and the guanylic acid synthetase gene.

Examples of the promoter that functions in a coryneform bacterium include such strong promoters as described above usable in coryneform bacteria. As the promoter, a high activity type of an existing promoter may be obtained by using various reporter genes, and used. For example, by making the −35 and −10 regions in a promoter region closer to a consensus sequence, activity of the promoter can be enhanced (International Patent Publication WO00/18935). Examples of the method for evaluating strength of a promoter and strong promoters are described in the paper of Goldstein et al. (Prokaryotic promoters in biotechnology, Biotechnol. Annu. Rev., 1, 105-128 (1995)) and so forth. Furthermore, it is known that substitution, insertion, or deletion of several nucleotides in a spacer region between the ribosome-binding site (RBS) and the start codon, especially in a sequence immediately upstream of the start codon (5′-UTR), significantly affects stability and translation efficiency of mRNA, and these sequences can also be modified.

The signal peptide can be a TorA signal peptide. The TorA signal peptide is a Tat-dependent signal peptide. The term “Tat-dependent signal peptide” refers to a signal peptide recognized by the Tat system. The term “Tat-dependent signal peptide” may specifically refer to a signal peptide that, upon being linked at the N-terminus of an objective protein, results in secretion of the protein by the Tat secretion system. The Tat-dependent signal peptide has a twin-arginine motif. Examples of the twin-arginine motif include R-R-X-#-# (#: hydrophobic residue) (SEQ ID NO: 45). Examples of the TorA signal peptide include signal peptides of TorA proteins (trimethylamine-N-oxidoreductase) of bacteria of the family Enterobacteriaceae such as E. coli. The amino acid sequence of the TorA signal peptide of E. coli is “MNNNDLFQASRRRFLAQLGGLTVAGMLGPSLLTPRRATA” (SEQ ID NO: 46).

The TorA signal peptide may be a variant of any of the TorA signal peptides exemplified above, so long as it contains a twin-arginine motif and the original function thereof is maintained. The above descriptions concerning conservative variants of the LepB protein and the lepB gene can be similarly applied to variants of the TorA signal peptide and the gene encoding it. For example, the TorA signal peptide may be a peptide having any of the amino acid sequences of the TorA signal peptides exemplified above, but including substitution, deletion, insertion, and/or addition of one or several amino acid residues at one or several positions. The number meant by the term “one or several” used for a variant of the TorA signal peptide is specifically, 1 to 7, 1 to 5, 1 to 3, or 1 to 2. Also, for example, the TorA signal peptide may be a peptide having an amino acid sequence having an identity of 80% or more, 90% or more, 95% or more, 97% or more, 99% or more, to the total amino acid sequence of any of the amino acid sequences of the TorA signal peptides exemplified above. The term “TorA signal peptide” includes not only the peptide of SEQ ID NO: 46, respectively, but also includes conservative variants thereof.

The expression “original function is maintained” used for the TorA signal peptide means that the peptide has a function as a Tat-dependent signal peptide. The expression “a peptide has a function as a Tat-dependent signal peptide” means that the peptide is recognized by the Tat system, and specifically, may mean that the peptide has a function of, upon being linked at the N-terminus of an objective protein, resulting in secretion of the protein by the Tat secretion system. Whether a peptide functions as the Tat-dependent signal peptide can be confirmed by, for example, confirming an increase in the secretory production amount of a protein linked with the peptide at the N-terminus due to enhancement of the Tat secretion system, or confirming a reduction in the secretory production amount of a protein linked with the peptide at the N-terminus due to deletion of the Tat secretion system.

The nucleic acid sequence encoding a TorA signal peptide is not particularly limited so long as it encodes any of such TorA signal peptides as mentioned above.

Examples of the heterologous protein to be produced by secretory production according to the method include, for example, physiologically active proteins, receptor proteins, antigenic proteins to be used as vaccines, enzymes, and any other proteins.

Examples of the enzymes include, for example, transglutaminase, protein glutaminase, isomaltodextranase, protease, endopeptidase, exopeptidase, aminopeptidase, carboxypeptidase, collagenase, chitinase, and so forth. Examples of transglutaminase include, for example, secretory-type transglutaminases of Actinomycetes such as Streptoverticillium mobaraense IFO 13819 (WO01/23591), Streptoverticillium cinnamoneum IFO 12852, Streptoverticillium griseocarneum IFO 12776, and Streptomyces lydicus (WO96/06931), and of filamentous fungi such as Oomycetes (WO96/22366). Examples of protein glutaminase include, for example, protein glutaminase of Chryseobacterium proteolyticum (WO2005/103278). Examples of isomaltodextranase include, for example, isomaltodextranase of Arthrobacter globiformis (WO2005/103278).

Examples of the physiologically active proteins include, for example, growth factors, hormones, cytokines, and antibody-related molecules.

Specific examples of the growth factors include, for example, epidermal growth factor (EGF), insulin-like growth factor-1 (IGF-1), transforming growth factor (TGF), nerve growth factor (NGF), brain-derived neurotrophic factor (BDNF), vascular endothelial growth factor (VEGF), granulocyte-colony stimulating factor (G-CSF), granulocyte-macrophage-colony stimulating factor (GM-CSF), platelet-derived growth factor (PDGF), erythropoietin (EPO), thrombopoietin (TPO), acidic fibroblast growth factor (aFGF or FGF1), basic fibroblast growth factor (bFGF or FGF2), keratinocyte growth factor (KGF-1 or FGF7, and, KGF-2 or FGF10), and hepatocyte growth factor (HGF).

Specific examples of the hormones include, for example, insulin, glucagon, somatostatin, human growth hormone (hGH), parathyroid hormone (PTH), calcitonin, and exenatide.

Specific examples of the cytokines include, for example, interleukins, interferons, and tumor necrosis factors (TNFs).

The growth factors, hormones, and cytokines may not be strictly distinguished from one another. For example, a physiologically active protein may be a one of a growth factor, hormone, or cytokine, or may be a plurality of those.

Furthermore, a physiologically active protein may be an intact protein, or may be a part of a protein. Examples of a part of a protein include, for example, a part having physiological activity. Specific examples of a part having physiological activity include, for example, teriparatide, a physiologically active peptide consisting of the N-terminal 34 amino acid residues of parathyroid hormone (PTH).

The term “antibody-related molecule” refers to a protein containing a molecular species having a single domain or a combination of two or more of the domains selected from a complete antibody. Examples of the domains from a complete antibody include heavy chain domains VH, CH1, CH2, and CH3, and light chain domains VL and CL. The antibody-related molecule may be a monomeric protein, or may be a multimeric protein, so long as it contains the above-mentioned molecular species. When the antibody-related molecule is a multimeric protein, it may be a homo-multimer having a single kind of subunit, or may be a hetero-multimer having two or more kinds of subunits. Specific examples of the antibody-related molecules include, for example, complete antibody, Fab, F(ab′), F(ab′)₂, Fc, dimer having a heavy chain (H chain) and a light chain (L chain), Fc-fusion protein, heavy chain (H chain), light chain (L chain), light chain Fv (scFv), sc(Fv)₂, disulfide-bonded Fv (sdFv), diabody, and VHH fragment (Nanobody (registered trademark)). More specific examples of the antibody-related molecules include, for example, Trastuzumab, Adalimumab, and Nivolumab.

The receptor proteins are not particularly limited. A receptor protein may be, for example, a receptor protein for any of physiologically active proteins and other physiologically active substances. Examples of the other physiologically active substances include, for example, neurotransmitters such as dopamine. Furthermore, a receptor protein may be an orphan receptor of which the corresponding ligand is not known.

The antigen proteins to be used as vaccines are not particularly limited, so long as they are proteins that can induce an immune response. An antigen protein can be appropriately selected depending on the intended object of the immune response.

In addition, examples of other proteins include liver-type fatty acid-binding protein (LFABP), fluorescent protein, immunoglobulin-binding protein, albumin, and extracellular protein. Examples of the fluorescent protein include Green Fluorescent Protein (GFP). Examples of the immunoglobulin-binding protein include Protein A, Protein G, and Protein L. Examples of albumin include human serum albumin.

Examples of the extracellular protein include fibronectin, vitronectin, collagen, osteopontin, laminin, and partial sequences thereof. Laminin is a protein having a heterotrimeric structure having an α chain, a β chain, and a γ chain. Examples of laminin include laminin of mammals. Examples of the mammals include primates such as human, monkey, and chimpanzee; rodents such as mouse, rat, hamster, and guinea pig; and other various mammals such as rabbit, horse, cattle, sheep, goat, pig, dog, and cat. Particular examples of the mammals include human. Examples of the subunit chains of laminin (i.e. a, (α, β, and γ chains) include 5 kinds of α chains (α1 to α5), 3 kinds of β chains (β1 to β3), and 3 kinds of γ chains (γ1 to γ3). Laminin constitutes various isoforms depending on combinations of these subunits. Specific examples of laminin include, for example, laminin 111, laminin 121, laminin 211, laminin 213, laminin 221, laminin 311, laminin 321, laminin 332, laminin 411, laminin 421, laminin 423, laminin 511, laminin 521, and laminin 523. Examples of the partial sequence of laminin include laminin E8, which is an E8 fragment of laminin. Laminin E8 is a protein having a heterotrimeric structure having an E8 fragment of α chain (α chain E8), an E8 fragment of β chain (β chain E8), and an E8 fragment of γ chain (γ chain E8). The subunit chains of laminin E8 (i.e. α chain E8, β chain E8, and γ chain E8) are also collectively referred to as “E8 subunit chains”. Examples of the E8 subunit chains includes E8 fragments of the laminin subunit chains exemplified above. Laminin E8 constitutes various isoforms depending on combinations of these E8 subunit chains. Specific examples of laminin E8 include, for example, laminin 111E8, laminin 121E8, laminin 211E8, laminin 221E8, laminin 332E8, laminin 421E8, laminin 411E8, laminin 511E8, and laminin 521E8.

A gene encoding the heterologous protein such as these proteins can be used as it is, or after being modified as required. A gene encoding the heterologous protein can be modified, for example, depending on a host to be used and/or for obtaining a desired activity. For example, a gene encoding the heterologous protein may be modified so that the amino acid sequence of the encoded heterologous protein include substitution, deletion, insertion, and/or addition of one or several amino acid residues. The above descriptions concerning variants of the LepB protein and the lepB gene can be similarly applied to the heterologous protein to be produced by secretory production by the method and the gene encoding it. A protein specified with the type of organism from which the protein is derived is not limited to proteins per se found in that organism, and shall also include proteins having any of the amino acid sequences of proteins found in that organism and variants thereof. That is, for example, the term “protein derived from human” is not limited to proteins per se found in human, and shall also include proteins having any of the amino acid sequences of proteins found in human and variants thereof. Furthermore, in the gene encoding the heterologous protein, any codon(s) may be replaced with respective equivalent codon(s) thereof. For example, the gene encoding the heterologous protein may be modified so as to have optimal codons according to the codon usage frequency of the chosen host.

The N-terminal region of the heterologous protein eventually obtained by the method as described herein may be the same as that of the natural protein, or may not be the same as that of the natural protein, so long as the TorA signal peptide has been completely removed. For example, the N-terminal region of the eventually obtained heterologous protein may be that of the natural protein including addition or deletion of one or several amino acid residues. Although the number of the “one or several” amino acid residues may differ depending on the full length or structure of the objective heterologous protein, specifically, it is 1 to 20, 1 to 10, 1 to 5, or 1 to 3.

Furthermore, the heterologous protein to be produced by secretory production may be a protein having a pro-structure moiety (proprotein). When the heterologous protein to be produced by secretory production is a proprotein, the heterologous protein to be eventually obtained may be the proprotein or may not be the proprotein. That is, the proprotein may be processed into the mature protein by cleavage of the pro-structure moiety. The cleavage can be attained with, for example, a protease. When a protease is used, generally, the proprotein is cleaved at a position substantially the same as that of the natural protein, or at exactly the same position as that of the natural protein so that the same mature protein as the natural mature protein is obtained, in view of the activity of the eventually obtained protein. Therefore, generally, a specific protease that cleaves the proprotein at such a position that the same protein as the naturally occurring mature protein is generated is most preferred. However, the N-terminal region of the heterologous protein to be eventually obtained may not be the same as that of the natural protein as described above. For example, depending on type, purpose of use etc. of the heterologous protein to be produced, a protein having an N-terminus longer or shorter by one to several amino acid residues compared with the natural protein may have more appropriate activity. Proteases usable in the present invention include, for example, commercially available proteases such as Dispase (produced by Boehringer Mannheim) as well as those obtainable from culture broth of a microorganism such as culture broth of actinomycetes. Such proteases may be used in an un-purified state, or may be used after purification to an appropriate purity as required.

The method for introducing the genetic construct into the coryneform bacterium is not particularly limited. The phrase “introduction of the genetic construct ” refers to making a host harbor the genetic construct. The phrase “introduction of the genetic construct” includes not only when the genetic construct that has been preliminarily constructed is collectively introduced into a host, but also includes when at least the heterologous protein gene is introduced into a host and the genetic construct is constructed in the host. In the bacterium, the genetic construct may be present on a vector that autonomously replicates separate from the chromosome such as a plasmid, or may be incorporated into the chromosome. The genetic construct can be introduced, for example, in the same manner as for introduction of a gene in methods for increasing the expression of a gene described above. In addition, for constructing the bacterium, introduction of the genetic structure used for the present invention, increase in the activity of the LepB protein, and other modifications can be performed in any order.

The genetic construct can be introduced into a host by using, for example, a vector that includes the genetic construct. For example, the genetic construct can be introduced into a host by ligating the genetic construct with a vector to construct an expression vector of the genetic construct, and transforming the host with the expression vector. Also, when the vector contains a promoter that functions in a coryneform bacterium, an expression vector of the genetic construct can be constructed by ligating the nucleic acid sequence encoding the fusion protein downstream from the promoter. The vector is not particularly limited so long as a vector autonomously replicable in a coryneform bacterium is chosen. The vector usable in a coryneform bacterium is as described above.

Furthermore, the genetic construct can be introduced into the chromosome of a host by using, for example, a transposon such as an artificial transposon. When a transposon is used, the genetic construct is introduced into the chromosome by homologous recombination or translocation ability of the transposon itself. Furthermore, the genetic construct can also be introduced into the chromosome of a host by other introduction methods utilizing homologous recombination. Examples of the introduction methods utilizing homologous recombination include, for example, methods utilizing a linear DNA, a plasmid having a temperature sensitive replication origin, a plasmid capable of conjugative transfer, a suicide vector not having a replication origin that functions in a host, and so forth. In addition, at least the heterologous protein gene may be introduced into the chromosome so that the genetic construct is present on the chromosome. In this case, some or all of the constituents contained in the genetic construct, other than the heterologous protein gene, may be inherently present on the chromosome of the host. Specifically, for example, by using a promoter sequence inherently present on the chromosome of the host as it is, and replacing only the gene ligated downstream from the promoter sequence with a nucleic acid sequence encoding the TorA signal peptide and an objective heterologous protein gene, the genetic construct can be incorporated into the chromosome, and the bacterium can be thereby constructed. A part of the genetic construct, such as the heterologous protein gene, can be introduced into the chromosome in the same manner as that for introduction of the genetic construct into the chromosome.

The genetic construct or a constituent thereof, such as promoter sequence, nucleic acid sequence encoding the TorA signal peptide, or nucleic acid sequence encoding a heterologous protein, can be obtained by, for example, cloning. Specifically, for example, the genetic construct can be obtained by obtaining an objective heterologous protein gene by cloning from an organism having the objective heterologous protein, and then subjecting the gene to modification such as introduction of the nucleic acid sequence encoding the TorA signal peptide and introduction of the promoter sequence. Furthermore, the genetic construct or a constituent thereof can also be obtained by chemical synthesis (Gene, 60(1), 115-127 (1987)). The obtained genetic construct or constituent thereof can be used as it is, or after being modified as required.

Furthermore, when two or more kinds of proteins are expressed, it is sufficient that the genetic constructs for secretory expression of the proteins are harbored by the bacterium so that secretory expression of the objective heterologous proteins can be attained. Specifically, for example, all the genetic constructs for secretory expression of the proteins may be present on a single expression vector, or present on the chromosome. Alternatively, the genetic constructs for secretory expression of the proteins may be separately present on a plurality of expression vectors, or may be separately present on one or more expression vectors and the chromosome. The “case where two or more kinds of proteins are expressed” refers to, for example, a case where two or more kinds of heterologous proteins are produced by secretory production, or a case where a hetero-multimeric protein is produced by secretory production.

The method for introducing the genetic construct into the coryneform bacterium is not particularly limited, and a generally used method, for example, the protoplast method (Gene, 39, 281-286 (1985)), the electroporation method (Bio/Technology, 7, 1067-1070 (1989)), the electric pulse method (Japanese Patent Laid-open (Kokai) No. 2-207791), and so forth can be used.

<2>Method for Producing Heterologous Protein

By culturing the bacterium obtained as described above to express a heterologous protein, a large amount of the heterologous protein secreted out of the cells is obtained.

The bacterium can be cultured according to a typically used method and conditions. For example, the bacterium can be cultured in a typical medium containing a carbon source, a nitrogen source, and inorganic ions. In order to obtain still higher proliferation, organic micronutrients such as vitamins and amino acids can also be added as required.

As the carbon source, carbohydrates such as glucose and sucrose, organic acids such as acetic acid, alcohols, and others can be used. As the nitrogen source, ammonia gas, aqueous ammonia, ammonium salts, and others can be used. As the inorganic ions, calcium ions, magnesium ions, phosphate ions, potassium ions, iron ions, and so forth are appropriately used as required. The culture is performed within appropriate ranges of pH 5.0 to 8.5 and 15 to 37° C. under aerobic conditions for 1 to 7 days. Furthermore, the culture conditions for L-amino acid production by coryneform bacteria and other conditions described for the methods for secretory production of a protein using a signal peptide can be used (refer to WO01/23591 and WO2005/103278). Furthermore, when an inducible promoter is used for expression of the heterologous protein, culture may also be performed by adding a promoter-inducing agent to the medium. By culturing the bacterium under such conditions, a large amount of the objective protein is produced in cells and efficiently secreted out of the cells. In addition, according to the method as described herein, the produced heterologous protein is secreted out of the cells, and therefore a protein that is generally lethal if it is accumulated in a large amount in cells of microorganisms, such as transglutaminases, can also be continuously produced without lethal effect.

The protein secreted in the medium according to the method can be separated and purified from the medium after the culture by a method well known to those skilled in the art. For example, after the cells are removed by centrifugation or the like, the protein can be separated and purified by a known appropriate method such as salting out, ethanol precipitation, ultrafiltration, gel filtration chromatography, ion exchange column chromatography, affinity chromatography, medium or high pressure liquid chromatography, reverse phase chromatography, and hydrophobic chromatography, or a combination of these. Furthermore, in a certain case, culture or culture supernatant may be used as it is. The protein secreted in the cell surface layer according to the method can also be separated and purified in the same manner as that for the case where the protein is secreted in the medium, after solubilizing it by a method well known to those skilled in the art such as elevation of salt concentration and use of a surfactant. Furthermore, in a certain case, the protein secreted in the cell surface layer may be used as, for example, an immobilized enzyme, without solubilizing it.

Secretory production of the objective heterologous protein can be confirmed by performing SDS-PAGE for the culture supernatant and/or a fraction containing the cell surface layer as a sample, and confirming the molecular weight of the separated protein band. Furthermore, secretory production of the objective heterologous protein can also be confirmed by performing Western blotting using antibodies for the culture supernatant and/or a fraction containing the cell surface layer as a sample (Molecular Cloning, Cold spring Harbor Laboratory Press, Cold Spring Harbor (USA), 2001). Furthermore, secretory production of the objective heterologous protein can also be confirmed by detecting an N-terminal amino acid sequence of the objective protein using a protein sequencer. Furthermore, secretory production of the objective heterologous protein can also be confirmed by determining the mass of the objective protein using a mass spectrometer. Furthermore, when the objective heterologous protein is an enzyme or a protein having a certain measurable physiological activity, secretory production of the objective heterologous protein can be confirmed by measuring enzymatic activity or the physiological activity of the objective protein in the culture supernatant and/or a fraction containing the cell surface layer as a sample.

### EXAMPLES

The present invention will be further specifically explained with reference to the following non-limiting examples.

### Example 1

### Construction of Amplification Vector of Signal Peptidase Gene (lepB) Derived from C. Glutamicum ATCC 13869

The genome sequence of the C. glutamicum ATCC 13869 strain and the nucleotide sequence of the lepB gene encoding Type I signal peptidase (LepB) have already been determined (GenBank Accession No. AP017557, NCBI locus_tag CGBL 0119390). The nucleotide sequence of the lepB gene derived from the C. glutamicum ATCC 13869 strain is shown as SEQ ID NO: 1, and the amino acid sequence of the LepB protein is shown as SEQ ID NO: 2.

PCR was carried out by using genomic DNA of the C. glutamicum ATCC 13869 strain prepared with PurElute™ Genomic DNA Kit (EdgeBio) as the template, in combination with primers of SEQ ID NOS: 3 and 4, to amplify a region containing a coding region of the lepB gene and a 5′-side upstream region thereof of about 0.2 kbp, to thereby obtain a DNA fragment of about 1.0 kbp. The primer of SEQ ID NO: 3 has been designed to contain a recognition sequence of restriction enzyme KpnI, and the primer of SEQ ID NO: 4 has been designed to contain recognition sequences of restriction enzymes ApaI and XbaI. PCR was carried out with PrimeSTAR(R) HS DNA Polymerase (Takara Bio), and the reaction conditions were according to the protocol recommended by the manufacturer.

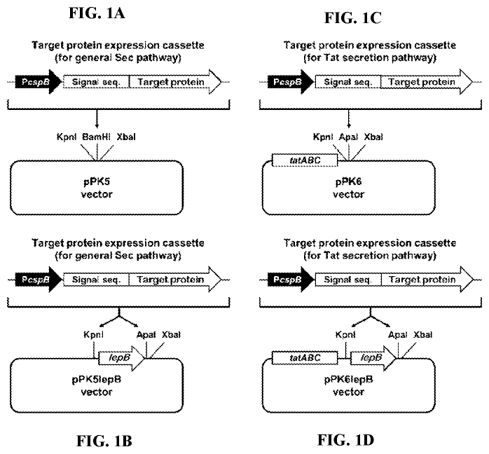

This PCR fragment was digested with restriction enzymes KpnI and XbaI, and inserted at the KpnI-XbaI site of pPK5 vector disclosed in WO2016/171224 by a ligation reaction, to obtain pPK5lepB, which is an amplification vector of lepB gene. Similarly, this PCR fragment was digested with restriction enzymes KpnI and ApaI, and inserted at the KpnI-ApaI site of pPK6 vector disclosed in WO2016/171224 by ligation reaction, to obtain pPK6lepB, which is an amplification vector of the tatABC secretion system genes and lepB gene. The ligation reaction was carried out with DNA Ligation Kit <Mighty Mix> (Takara Bio), and the reaction conditions were according to the protocol recommended by the manufacturer. The pPK5 vector is a vector obtained by modifying Nael restriction enzyme site in pPK4 vector disclosed in Japanese Patent Laid-open (Kokai) No. 9-322774, and the pPK6 is an amplification vector of TatABC secretion system obtained by inserting tatABC genes into the pPK5 vector (FIG. 1, panels A and C). As a result of nucleotide sequencing of the inserted fragments, it was confirmed that the lepB gene as expected was inserted into each vector. Nucleotide sequencing was carried out with BigDye(R) Terminator v3.1 Cycle Sequencing Kit (Applied Biosystems) and 3130 Genetic Analyzer (Applied Biosystems).

When subcloning an expression cassette of an objective protein using pPK5lepB or pPK6lepB as an expression vector, the insertion order of the lepB gene and the expression cassette of the objective protein can be controlled by using KpnI site or ApaI-XbaI site (FIG. 1, panels B and D).

### Example 2

### Evaluation of Effect of lepB Gene-Amplification on Secretory Expression of Transglutaminase Comprising a Pro-Structure Moiety (PTG) using TorA Signal Sequence

(1) Secretory expression of PTG using TorA signal peptide under existing (non-lepB gene-amplified) condition

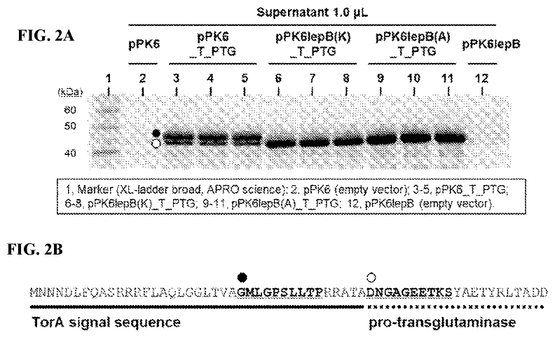

The YDK010::phoS(W302C)/pPK6_T_PTG strain disclosed in WO2016/171224 was cultured on MMTG liquid medium (120 g of glucose, 3 g of MgSO₄.7H₂O, 30 g of (NH₄)₂SO₄, 1.5 g of KH₂PO₄, 0.03 g of FeSO₄.7H₂O, 0.03 g of MnSO₄.5H₂O, 0.45 mg of thiamine hydrochloride, 0.45 mg of biotin, 0.15 g of DL-methionine, 0.2 g (as total nitrogen) of soybean hydrolysate solution obtained with HCl, and 50 g of CaCO₃, filled up with water to 1 L, and adjusted to pH7.0) containing 25 mg/L of kanamycin at 30° C. for 72 hr. Incidentally, pPK6_T_PTG is a secretory expression vector of transglutaminase including a pro-structure moiety (PTG) constructed from the amplification vector of tatABC genes pPK6, and also is a plasmid containing the promoter of cspB gene of the C. glutamicum ATCC13869 strain, DNA encoding the TorA signal peptide of E. coli expressively linked downstream from the promoter, and PTG gene of Streptomyces mobaraense linked thereto so as to be expressed as a fusion protein with the signal peptide (WO2016/171224). The YDK010::phoS(W302C) strain is a strain obtained from the YDK010 strain (WO2002/081694), which is a cell surface layer protein CspB-deficient strain of C. glutamicum AJ12036 (FERM BP-734), by introducing PhoS(W302C) mutation into phoS gene on the chromosome thereof (WO2016/171224). After completion of the culture, 1.0 μL of the culture supernatant obtained by centrifuging the culture broth was subjected to reduced SDS-PAGE using MULTIGEL(R) II mini 15/20 (Cosmo Bio), and then staining was carried out with SYPRO(R) Orange (Thermo Fisher Scientific). As a result, two bands were mainly detected (FIG. 2, panel A, lanes 3-5). As a result of analysis of the N-terminal amino acid sequence of each band, an amino acid sequence indicating that C-terminal side 15 residues of the TorA signal sequence remains at the N-terminus of PTG (GMLGPSLLTP; SEQ ID NO: 47) was observed for the high molecular weight-side band, whereas the N-terminal amino acid sequence of PTG (DNGAGEETKS; SEQ ID NO: 48) was observed for the low molecular weight-side band (FIG. 2, panel B). That is, it was revealed that a mis-cleaved form of C-terminal side 15 residues of the TorA signal peptide significantly remains in secretory production of PTG using the TorA signal peptide.

(2) Construction of Secretory Expression Vector of PTG Using pPK6lepB Vector

PCR was carried out by using the PTG secretory expression vector pPK6_T_PTG disclosed in WO2016/171224 as the template, in combination with primers of SEQ ID NOS: 5 and 6 or in combination with primers of SEQ ID NOS: 7 and 8, to amplify a DNA fragment of about 1.9 kbp containing an expression cassette of cspB promoter-TorA signal peptide-PTG. The primers of SEQ ID NOS: 5 and 6 have been designed to contain a recognition sequence of restriction enzyme KpnI, and the primers of SEQ ID NOS: 7 and 8 have been designed to contain recognition sequences of restriction enzymes ApaI. PCR was carried out with PrimeSTAR(R) HS DNA Polymerase (Takara Bio), and the reaction conditions were according to the protocol recommended by the manufacturer.

These DNA fragments were inserted at KpnI or ApaI site of pPK6lepB vector constructed in Example 1 by infusion reaction, to obtain pPK6lepB(K)_T_PTG and pPK6lepB(A)_T_PTG, which are co-expression plasmids of tatABC secretion system genes, lepB gene, and PTG gene fused with the TorA signal sequence (FIG. 1, panel D). The letters (K) and (A) in the plasmid names represent the restriction enzyme sites of pPK6lepB vector inserted with the expression cassette of PTG (KpnI site and ApaI site), respectively. The infusion reaction was carried out with In-Fusion(R) HD Cloning Kit (Takara Bio), and the reaction conditions were according to the protocol recommended by the manufacturer. As a result of nucleotide sequencing of the inserted fragments, it was confirmed that the gene as expected was inserted into each vector. Nucleotide sequencing was carried out with BigDye(R) Terminator v3.1 Cycle Sequencing Kit (Applied Biosystems) and 3130 Genetic Analyzer (Applied Biosystems).

(3) Secretory Expression of PTG Using TorA Signal Peptide Under lepB Gene-Amplified Condition

The YDK010::phoS(W302C) strain disclosed in WO2016/171224 was transformed with the PTG secretory expression plasmids pPK6lepB(K)_T_PTG and pPK6lepB(A)_T_PTG constructed in Example 2(2), to thereby obtain strains YDK010::phoS(W302C)/pPK6lepB(K)_T_PTG and YDK010::phoS(W302C)/pPK6lepB(A)_T_PTG. The obtained transformants were each cultured on MMTG liquid medium containing 25 mg/L of kanamycin at 30° C. for 72 hr. After completion of the culture, 1.0 μL of the culture supernatant obtained by centrifuging the culture broth was subjected to reduced SDS-PAGE using MULTIGEL(R) II mini 15/20 (Cosmo Bio), and then staining was carried out with SYPRO(R) Orange (Thermo Fisher Scientific). As a result, two bands were mainly detected in the strain introduced with pPK6P_T_PTG, in which lepB gene was not amplified, whereas the high molecular weight-side band was decreased so as to be scarcely detected and the low molecular weight-side band was increased in both the strains introduced with pPK6lepB(K)_T_PTG and pPK6lepB(A)_T_PTG, in which lepB gene was amplified (FIG. 2, panel A, lanes 6-11). As a result of analysis of the N-terminal amino acid sequence, the N-terminal amino acid sequence of PTG (DNGAGEETKS; SEQ ID NO: 48) was observed for the low molecular weight-side band. That is, it was revealed that although a mis-cleaved form of C-terminal side 15 residues of the TorA signal peptide significantly remains in secretory production of PTG using the TorA signal peptide under non-lepB gene-amplified condition, amplification of lepB gene can promote processing of the TorA signal peptide, to thereby significantly improve production efficiency of PTG having the correct N-terminal amino acid sequence.

Example 3: Evaluation of the effect of lepB gene-amplification on secretory expression of protein glutaminase including a pro-structure moiety (PPG) using the TorA signal sequence

(1) Secretory Expression of PPG Using the TorA Signal Peptide Under Existing (Non-lepB Gene-Amplified) Conditions

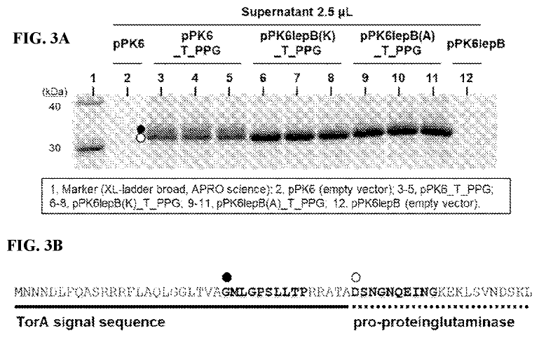

The YDK010::phoS(W302C)/pPK6_T_PPG strain disclosed in WO2016/171224 was cultured on MMTG liquid medium containing 25 mg/L of kanamycin at 30° C. for 72 hr. Incidentally, pPK6_T_PPG is a secretory expression vector of protein glutaminase including a pro-structure moiety (PPG) constructed from the amplification vector of tatABC genes pPK6, and also is a plasmid containing the promoter of cspB gene of the C. glutamicum ATCC13869 strain, DNA encoding the TorA signal peptide of E. coli expressively linked downstream from the promoter, and PPG gene of Chryseobacterium proteolyticum linked thereto so as to be expressed as a fusion protein with the signal peptide (WO2016/171224). After completion of the culture, 2.5 μL of the culture supernatant obtained by centrifuging the culture broth was subjected to reduced SDS-PAGE using MULTIGEL(R) II mini 15/20 (Cosmo Bio), and then staining was carried out with SYPRO(R) Orange (Thermo Fisher Scientific). As a result, two bands were mainly detected (FIG. 3, panel A, lanes 3-5). As a result of analysis of the N-terminal amino acid sequence of each band, an amino acid sequence indicating that C-terminal side 15 residues of the TorA signal sequence remains at the N-terminus of PPG (GMLGPSLLTP; SEQ ID NO: 47) was observed for the high molecular weight-side band, whereas the N-terminal amino acid sequence of PPG (DSNGNQEING; SEQ ID NO: 49) was observed for the low molecular weight-side band (FIG. 3, panel B). That is, it was revealed that a mis-cleaved form of C-terminal side 15 residues of the TorA signal peptide significantly remains in secretory production of PPG using the TorA signal peptide.

(2) Construction of Secretory Expression Vector of PPG Using pPK6lepB Vector

PCR was carried out by using the PPG secretory expression vector pPK6_T_PPG disclosed in WO2016/171224 as the template, in combination with primers of SEQ ID NOS: 5 and 9 or in combination with primers of SEQ ID NOS: 7 and 10, to amplify a DNA fragment of about 1.6 kbp containing an expression cassette of cspB promoter-TorA signal sequence-PPG. The primers of SEQ ID NOS: 5 and 9 have been designed to contain a recognition sequence of restriction enzyme KpnI, and the primers of SEQ ID NOS: 7 and 10 have been designed to contain recognition sequences of restriction enzymes ApaI. PCR was carried out with PrimeSTAR(R) HS DNA Polymerase (Takara Bio), and the reaction conditions were according to the protocol recommended by the manufacturer.

These DNA fragments were inserted at KpnI or ApaI site of pPK6lepB vector constructed in Example 1 by infusion reaction, to obtain pPK6lepB(K)_T_PPG and pPK6lepB(A)_T_PPG, which are co-expression plasmids of tatABC secretion system genes, lepB gene, and PPG gene fused with the TorA signal sequence (FIG. 1, panel D). The letters (K) and (A) in the plasmid names represent the restriction enzyme sites of pPK6lepB vector inserted with the expression cassette of PPG (KpnI site and ApaI site), respectively. The infusion reaction was carried out with In-Fusion(R) HD Cloning Kit (Takara Bio), and the reaction conditions were according to the protocol recommended by the manufacturer. As a result of nucleotide sequencing of the inserted fragments, it was confirmed that the gene as expected was inserted into each vector. Nucleotide sequencing was carried out with BigDye(R) Terminator v3.1 Cycle Sequencing Kit (Applied Biosystems) and 3130 Genetic Analyzer (Applied Biosystems).

(3) Secretory Expression of PPG Using TorA Signal Peptide Under lepB Gene-Amplified Condition

The YDK010::phoS(W302C) strain disclosed in WO2016/171224 was transformed with the PPG secretory expression plasmids pPK6lepB(K)_T_PPG and pPK6lepB(A)_T_PPG constructed in Example 3(2), to thereby obtain strains YDK010::phoS(W302C)/pPK6lepB(K)_T_PPG and YDK010::phoS(W302C)/pPK6lepB(A)_T_PPG. The obtained transformants were each cultured on MMTG liquid medium containing 25 mg/L of kanamycin at 30° C. for 72 hr. After completion of the culture, 2.5 μL of the culture supernatant obtained by centrifuging the culture broth was subjected to reduced SDS-PAGE using MULTIGEL(R) II mini 15/20 (Cosmo Bio), and then staining was carried out with SYPRO(R) Orange (Thermo Fisher Scientific). As a result, two bands were mainly detected in the strain introduced with pPK6_T_PPG, in which lepB gene was not amplified, whereas the high molecular weight-side band was decreased so as to be scarcely detected and the low molecular weight-side band was increased in both the strains introduced with pPK6lepB(K)_T_PPG and pPK6lepB(A)_T_PPG, in which lepB gene was amplified (FIG. 3, panel A, lanes 6-11). As a result of analysis of the N-terminal amino acid sequence, the N-terminal amino acid sequence of PPG (DSNGNQEING; SEQ ID NO: 49) was observed for the low molecular weight-side band. That is, it was revealed that although a mis-cleaved form of C-terminal side 15 residues of the TorA signal peptide significantly remains in secretory production of PPG using the TorA signal peptide under non-lepB gene-amplified condition, amplification of lepB gene can promote processing of the TorA signal peptide, to thereby significantly improve production efficiency of PPG having the correct N-terminal amino acid sequence.

### Example 4

### Evaluation of Effect of lepB Gene-Amplification on Secretory Expression of Beta-Lactamase (Bla) Using TorA Signal Sequence

(1) Construction of Co-Expression Plasmid of tatABC Genes Encoding Tat Secretion System and Gene Encoding Bla Added with TorA Signal Sequence

The amino acid sequence of class A broad-spectrum beta-lactamase TEM-116 belonging to the TEM-type, which is one of beta-lactamases (hereinafter referred to as “Bla”), has already been determined (GenBank Accession No. WP_000027050). The amino acid sequence of 263 residues corresponding to positions 24-286 of this amino acid sequence, excluding the N-terminal signal sequence, is shown as SEQ ID NO: 11. Considering the codon usage frequency of C. glutamicum, a nucleotide sequence encoding Bla of SEQ ID NO: 11 was designed. The designed nucleotide sequence is shown as SEQ ID NO: 12.

Then, an expression cassette of a fusion protein of the TorA signal peptide and Bla, in which a DNA encoding 39 amino acid residues consisting of a signal peptide of TorA protein derived from E. coli (UniProt Accession number: P33225) and the DNA of SEQ ID NO: 12 were linked downstream of the promoter of cspB gene of the C. glutamicum ATCC 13869 strain, and KpnI site and ApaI site were further added at the 5′ -side and 3′ -side termini, respectively, was totally synthesized. The synthesized DNA fragment was inserted at KpnI-ApaI site of pPK6 vector disclosed in WO2016/171224, to construct pPK6_T_Bla, which is a secretory expression plasmid of Bla using the TorA signal sequence (FIG. 1, panel C). As a result of nucleotide sequencing of the inserted fragment, it was confirmed that the gene encoding the expression cassette of Bla gene as intended was constructed. Nucleotide sequencing was carried out with BigDye(R) Terminator v3.1 Cycle Sequencing Kit (Applied Biosystems) and 3130 Genetic Analyzer (Applied Biosystems).

(2) Secretory Expression of Bla Using TorA Signal Peptide Under Non-lepB Gene-Amplified Condition

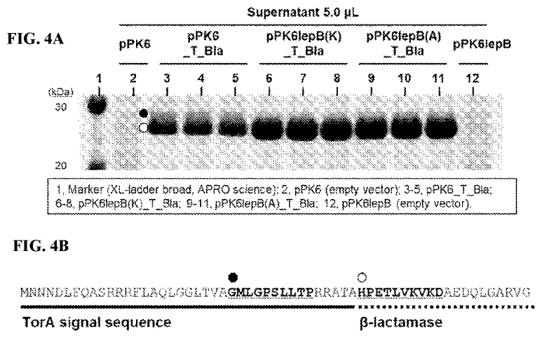

The YDK010::phoS(W302C) strain disclosed in WO2016/171224 was transformed with the Bla secretory expression plasmid pPK6_T_Bla constructed in Example 4(1), to thereby obtain a strain YDK010::phoS(W302C)/pPK6_T_Bla. The obtained transformant was cultured on MMTG liquid medium containing 25 mg/L of kanamycin at 30° C. for 72 hr. After completion of the culture, 5.0 μL of the culture supernatant obtained by centrifuging the culture broth was subjected to reduced SDS-PAGE using NuPAGE(R) 4-12% Bis-Tirs Gel (Thermo Fisher Scientific), and then staining was carried out with SYPRO(R) Ruby (Thermo Fisher Scientific). As a result, two bands were mainly detected (FIG. 4, panel A, lanes 3-5). As a result of analysis of the N-terminal amino acid sequence of each band, an amino acid sequence indicating that C-terminal side 15 residues of the TorA signal sequence remains at the N-terminus of Bla (GMLGPSLLTP; SEQ ID NO: 47) was observed for the high molecular weight-side band, whereas the N-terminal amino acid sequence of Bla (HPETLVKVKD; SEQ ID NO: 50) was observed for the low molecular weight-side band (FIG. 4, panel B). That is, it was revealed that a mis-cleaved form of C-terminal side 15 residues of the TorA signal peptide remains in secretory production of Bla using the TorA signal peptide.

(3) Construction of Secretory Expression Vector of Bla Using pPK6lepB Vector

PCR was carried out by using the Bla secretory expression vector pPK6_T_Bla constructed in Example 4(1) as the template, in combination with primers of SEQ ID NOS: 5 and 13 or in combination with primers of SEQ ID NOS: 7 and 14, to amplify a DNA fragment of about 1.5 kbp containing an expression cassette of cspB promoter-TorA signal sequence-Bla. The primers of SEQ ID NOS: 5 and 13 have been designed to contain a recognition sequence of restriction enzyme KpnI, and the primers of SEQ ID NOS: 7 and 14 have been designed to contain recognition sequences of restriction enzymes ApaI. PCR was carried out with PrimeSTAR(R) HS DNA Polymerase (Takara Bio), and the reaction conditions were according to the protocol recommended by the manufacturer.

These DNA fragments were inserted at KpnI or ApaI site of pPK6lepB vector constructed in Example 1 by infusion reaction, to obtain pPK6lepB(K)_T_Bla and pPK6lepB(A)_T_Bla, which are co-expression plasmids of tatABC secretion system genes, lepB gene, and Bla gene fused with the TorA signal sequence (FIG. 1, panel D). The letters (K) and (A) in the plasmid names represent the restriction enzyme sites of pPK6lepB vector inserted with the expression cassette of Bla (KpnI site and ApaI site), respectively. The infusion reaction was carried out with In-Fusion(R) HD Cloning Kit (Takara Bio), and the reaction conditions were according to the protocol recommended by the manufacturer. As a result of nucleotide sequencing of the inserted fragments, it was confirmed that the gene as expected was inserted into each vector. Nucleotide sequencing was carried out with BigDye(R) Terminator v3.1 Cycle Sequencing Kit (Applied Biosystems) and 3130 Genetic Analyzer (Applied Biosystems).

(4) Secretory Expression of Bla Using TorA Signal Peptide Under lepB Gene-Amplified Condition

The YDK010::phoS(W302C) strain disclosed in WO2016/171224 was transformed with the Bla secretory expression plasmids pPK6lepB(K)_T_Bla and pPK6lepB(A)_T_Bla constructed in Example 4(3), to thereby obtain strains YDK010::phoS(W302C)/pPK6lepB(K)_T_Bla and YDK010::phoS(W302C)/pPK6lepB(A)_T_Bla. The obtained transformants were each cultured on MMTG liquid medium containing 25 mg/L of kanamycin at 30° C. for 72 hr. After completion of the culture, 5.0 μL of the culture supernatant obtained by centrifuging the culture broth was subjected to reduced SDS-PAGE using NuPAGE(R) 4-12% Bis-Tirs Gel (Thermo Fisher Scientific), and then staining was carried out with SYPRO(R) Ruby (Thermo Fisher Scientific). As a result, two bands were mainly detected in the strain introduced with pPK6_T_Bla, in which lepB gene was not amplified, whereas the high molecular weight-side band was decreased so as to be scarcely detected and the low molecular weight-side band was increased in both the strains introduced with pPK6lepB(K)_T_Bla and pPK6lepB(A)_T_Bla, in which lepB gene was amplified (FIG. 4, panel A, lanes 6-11). As a result of analysis of the N-terminal amino acid sequence, the N-terminal amino acid sequence of Bla (HPETLVKVKD; SEQ ID NO: 50) was observed for the low molecular weight-side band. That is, it was revealed that although a mis-cleaved form of C-terminal side 15 residues of the TorA signal peptide remains in secretory production of Bla using the TorA signal peptide under non-lepB gene-amplified condition, amplification of lepB gene can promote processing of the TorA signal peptide, to thereby significantly improve production efficiency of Bla having the correct N-terminal amino acid sequence.

### Example 5

### Evaluation of Effect of lepB Gene-Amplification on Secretory Expression of Thioredoxin (Trx) Using TorA Signal Sequence

(1) Construction of Co-Expression Plasmid of tatABC Genes Encoding Tat Secretion System and Gene Encoding Trx Added with TorA Signal Sequence

The amino acid sequence of TrxA protein of E. coli, which is one of thioredoxins (hereinafter referred to as “Trx”), has already been determined (GenBank Accession No. WP_001323277). The amino acid sequence of 109 residues of a modified Trx, which is obtained by removing N-terminal 19 residues from this amino acid sequence and then adding one residue of Ala at the N-terminus, is shown as SEQ ID NO: 15. Considering the codon usage frequency of C. glutamicum, a nucleotide sequence encoding Trx of SEQ ID NO: 15 was designed. The designed nucleotide sequence is shown as SEQ ID NO: 16.

Then, an expression cassette of a fusion protein of the TorA signal peptide and Trx, in which a DNA encoding 39 amino acid residues consisting of a signal peptide of TorA protein derived from E. coli (UniProt Accession number: P33225) and the DNA of SEQ ID NO: 16 were linked downstream of the promoter of cspB gene of the C. glutamicum ATCC 13869 strain, and KpnI site and ApaI site were further added at the 5′-side and 3′-side termini, respectively, was totally synthesized. The synthesized DNA fragment was inserted at KpnI-ApaI site of pPK6 vector disclosed in WO2016/171224, to construct pPK6_T_Trx, which is a secretory expression plasmid of Trx (FIG. 1, panel C). As a result of nucleotide sequencing of the inserted fragment, it was confirmed that the gene encoding the expression cassette of Trx gene as intended was constructed. Nucleotide sequencing was carried out with BigDye(R) Terminator v3.1 Cycle Sequencing Kit (Applied Biosystems) and 3130 Genetic Analyzer (Applied Biosystems).

(2) Secretory Expression of Trx Using TorA Signal Peptide Under Non-lepB Gene-Amplified Condition

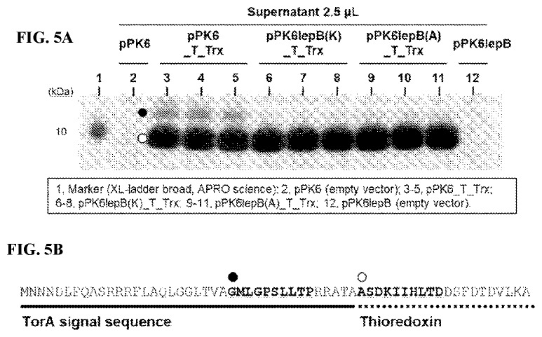

The YDK010::phoS(W302C) strain disclosed in WO2016/171224 was transformed with the Trx secretory expression plasmid pPK6_T_Trx constructed in Example 5(1), to thereby obtain a strain YDK010::phoS(W302C)/pPK6_T_Trx. The obtained transformant was cultured on MMTG liquid medium containing 25 mg/L of kanamycin at 30° C. for 72 hr. After completion of the culture, 2.5 μL of the culture supernatant obtained by centrifuging the culture broth was subjected to reduced SDS-PAGE using NuPAGE(R) 12% Bis-Tirs Gel (Thermo Fisher Scientific), and then staining was carried out with SYPRO(R) Ruby (Thermo Fisher Scientific). As a result, two bands were mainly detected (FIG. 5, panel A, lanes 3-5). As a result of analysis of the N-terminal amino acid sequence of each band, an amino acid sequence indicating that C-terminal side 15 residues of the TorA signal sequence remains at the N-terminus of Trx (GMLGPSLLTP; SEQ ID NO: 47) was observed for the high molecular weight-side band, whereas the N-terminal amino acid sequence of Trx (ASDKIIHLTD; SEQ ID NO: 51) was observed for the low molecular weight-side band (FIG. 5, panel B). That is, it was revealed that a mis-cleaved form of C-terminal side 15 residues of the TorA signal peptide remains in secretory production of Trx using the TorA signal peptide.

(3) Construction of Secretory Expression Vector of Trx Using pPK6lepB Vector

PCR was carried out by using the Trx secretory expression vector pPK6_T_Trx constructed in Example 5(1) as the template, in combination with primers of SEQ ID NOS: 5 and 17 or in combination with primers of SEQ ID NOS: 7 and 18, to amplify a DNA fragment of about 1.1 kbp containing an expression cassette of cspB promoter-TorA signal sequence-Trx. The primers of SEQ ID NOS: 5 and 17 have been designed to contain a recognition sequence of restriction enzyme KpnI, and the primers of SEQ ID NOS: 7 and 18 have been designed to contain recognition sequences of restriction enzymes ApaI. PCR was carried out with PrimeSTAR(R) HS DNA Polymerase (Takara Bio), and the reaction conditions were according to the protocol recommended by the manufacturer.

These DNA fragments were inserted at KpnI or ApaI site of pPK6lepB vector constructed in Example 1 by infusion reaction, to obtain pPK6lepB(K)_T_Trx and pPK6lepB(A)_T_Trx, which are co-expression plasmids of tatABC secretion system genes, lepB gene, and Trx gene fused with the TorA signal sequence (FIG. 1, panel D). The letters (K) and (A) in the plasmid names represent the restriction enzyme sites of pPK6lepB vector inserted with the expression cassette of Trx (KpnI site and ApaI site), respectively. The infusion reaction was carried out with In-Fusion(R) HD Cloning Kit (Takara Bio), and the reaction conditions were according to the protocol recommended by the manufacturer. As a result of nucleotide sequencing of the inserted fragments, it was confirmed that the gene as expected was inserted into each vector. Nucleotide sequencing was carried out with BigDye(R) Terminator v3.1 Cycle Sequencing Kit (Applied Biosystems) and 3130 Genetic Analyzer (Applied Biosystems).

(4) Secretory Expression of Trx Using TorA Signal Peptide Under lepB Gene-Amplified Condition

The YDK010::phoS(W302C) strain disclosed in WO2016/171224 was transformed with the Trx secretory expression plasmids pPK6lepB(K)_T_Trx and pPK6lepB(A)_T_Trx constructed in Example 5(3), to thereby obtain strains YDK010::phoS(W302C)/pPK6lepB(K)_T_Trx and YDK010::phoS(W302C)/pPK6lepB(A)_T_Trx. The obtained transformants were each cultured on MMTG liquid medium containing 25 mg/L of kanamycin at 30° C. for 72 hr. After completion of the culture, 2.5 μL of the culture supernatant obtained by centrifuging the culture broth was subjected to reduced SDS-PAGE using NuPAGE(R) 12% Bis-Tirs Gel (Thermo Fisher Scientific), and then staining was carried out with SYPRO(R) Ruby (Thermo Fisher Scientific). As a result, two bands were mainly detected in the strain introduced with pPK6_T_Trx, in which lepB gene was not amplified, whereas the high molecular weight-side band was decreased so as to be scarcely detected and the low molecular weight-side band was increased in both the strains introduced with pPK6lepB(K)_T_Trx and pPK6lepB(A)_T_Trx, in which lepB gene was amplified (FIG. 5, panel A, lanes 6-11). As a result of analysis of the N-terminal amino acid sequence, the N-terminal amino acid sequence of Trx (ASDKIIHLTD; SEQ ID NO: 51) was observed for the low molecular weight-side band. That is, it was revealed that although a mis-cleaved form of C-terminal side 15 residues of the TorA signal peptide remains in secretory production of Trx using the TorA signal peptide under non-lepB gene-amplified condition, amplification of lepB gene can promote processing of the TorA signal peptide, to thereby significantly improve production efficiency of Trx having the correct N-terminal amino acid sequence.

### Example 6

### Evaluation of Effect of lepB Gene-Amplification on Secretory Expression of Liver-Type Fatty Acid-Binding Protein (LFABP) Using TorA Signal Sequence

(1) Construction of Co-Expression Plasmid of tatABC Genes Encoding Tat Secretion System and Gene Encoding LFABP Added with TorA Signal Sequence

The amino acid sequence of Liver-type fatty acid-binding protein (hereinafter referred to as “LFABP”) of human has already been determined (GenBank Accession No. NP_001434). This amino acid sequence of 127 residues is shown as SEQ ID NO: 19. Considering the codon usage frequency of C. glutamicum, a nucleotide sequence encoding LFABP of SEQ ID NO: 19 was designed. The designed nucleotide sequence is shown as SEQ ID NO: 20.

Then, an expression cassette of a fusion protein of the TorA signal peptide and LFABP, in which a DNA encoding 39 amino acid residues consisting of a signal peptide of TorA protein derived from E. coli(UniProt Accession number: P33225) and the DNA of SEQ ID NO: 20 were linked downstream of the promoter of cspB gene of the C. glutamicum ATCC 13869 strain, and KpnI site and ApaI site were further added at the 5′-side and 3′-side termini, respectively, was totally synthesized. The synthesized DNA fragment was inserted at KpnI-ApaI site of pPK6 vector disclosed in WO2016/171224, to construct pPK6_T_LFABP, which is a secretory expression plasmid of LFABP using the TorA signal sequence (FIG. 1, panel C). As a result of nucleotide sequencing of the inserted fragment, it was confirmed that the gene encoding the expression cassette of LFABP gene as intended was constructed. Nucleotide sequencing was carried out with BigDye(R) Terminator v3.1 Cycle Sequencing Kit (Applied Biosystems) and 3130 Genetic Analyzer (Applied Biosystems).

(2) Secretory Expression of LFABP Using TorA Signal Peptide Under Non-lepB Gene-Amplified Condition

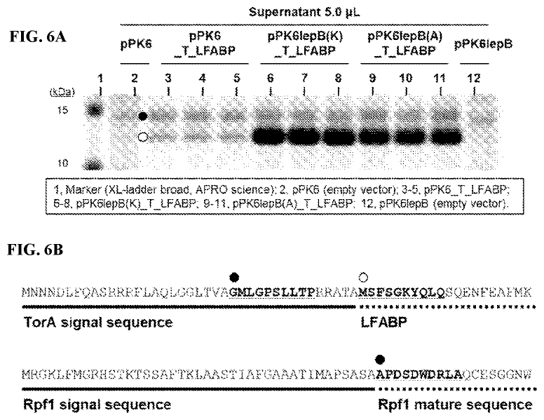

The YDK010::phoS(W302C) strain disclosed in WO2016/171224 was transformed with the LFABP secretory expression plasmid pPK6_T_LFABP constructed in Example 6(1), to thereby obtain a strain YDK010::phoS(W302C)/pPK6_T_LFABP. The obtained transformant was cultured on MMTG liquid medium containing 25 mg/L of kanamycin at 30° C. for 72 hr. After completion of the culture, 5.0 μL of the culture supernatant obtained by centrifuging the culture broth was subjected to reduced SDS-PAGE using NuPAGE(R) 4-12% Bis-Tirs Gel (Thermo Fisher Scientific), and then staining was carried out with SYPRO(R) Ruby (Life Technologies). As a result, two bands were mainly detected (FIG. 6, panel A, lanes 3-5). Incidentally, since the high molecular weight-side band was detected also in a culture supernatant of the YDK010::phoS(W302C)/pPK6 strain used as a negative control (FIG. 6, panel A, lane 2), a protein of a host, C. glutamicum, was presumed to be contained therein. As a result of analysis of the N-terminal amino acid sequence of each band, an amino acid sequence indicating that C-terminal side 15 residues of the TorA signal sequence remains at the N-terminus of LFABP (GMLGPSLLTP; SEQ ID NO: 47) and the N-terminal sequence of a mature protein of resuscitation-promoting factor Rpf1 of C. glutamicum (GenBank Accession No. AP017557, NCBI locus tag CGBL 0109030) (APDSDWDRLA; SEQ ID NO: 52) were detected in the high molecular weight-side band, and hence, the high molecular weight-side band was confirmed to be a mixture band consisting mainly of these two kinds of proteins (FIG. 6, panel B). Incidentally, no amino acid sequence indicating that C-terminal-side partial sequence of the Rpf1 signal sequence remains was detected, and hence, it was confirmed that the full-length signal peptide was correctly cleaved as for Rpf1. By contrast, the N-terminal amino acid sequence of LFABP (MSFSGKYQLQ; SEQ ID NO: 53) was observed for the low molecular weight-side band (FIG. 6, panel B). That is, it was revealed that a mis-cleaved form of C-terminal side 15 residues of the TorA signal peptide remains in secretory production of LFABP using the TorA signal peptide.

(3) Construction of Secretory Expression Vector of LFABP Using pPK6lepB Vector

PCR was carried out by using the LFABP secretory expression vector pPK6_T_LFABP constructed in Example 6(1) as the template, in combination with primers of SEQ ID NOS: 5 and 21 or in combination with primers of SEQ ID NOS: 7 and 22, to amplify a DNA fragment of about 1.1 kbp containing an expression cassette of cspB promoter-TorA signal sequence-LFABP. The primers of SEQ ID NOS: 5 and 21 have been designed to contain a recognition sequence of restriction enzyme KpnI, and the primers of SEQ ID NOS: 7 and 22 have been designed to contain recognition sequences of restriction enzymes ApaI. PCR was carried out with PrimeSTAR(R) HS DNA Polymerase (Takara Bio), and the reaction conditions were according to the protocol recommended by the manufacturer.

These DNA fragments were inserted at KpnI or ApaI site of pPK6lepB vector constructed in Example 1 by infusion reaction, to obtain pPK6lepB(K)_T_LFABP and pPK6lepB(A)_T_LFABP, which are co-expression plasmids of tatABC secretion system genes, lepB gene, and LFABP gene fused with the TorA signal sequence (FIG. 1, panel D). The letters (K) and (A) in the plasmid names represent the restriction enzyme sites of pPK6lepB vector inserted with the expression cassette of LFABP (KpnI site and ApaI site), respectively. The infusion reaction was carried out with In-Fusion(R) HD Cloning Kit (Takara Bio), and the reaction conditions were according to the protocol recommended by the manufacturer. As a result of nucleotide sequencing of the inserted fragments, it was confirmed that the gene as expected was inserted into each vector. Nucleotide sequencing was carried out with BigDye(R) Terminator v3.1 Cycle Sequencing Kit (Applied Biosystems) and 3130 Genetic Analyzer (Applied Biosystems).

(4) Secretory Expression of LFABP Using TorA Signal Peptide Under lepB Gene-Amplified Condition

The YDK010::phoS(W302C) strain disclosed in WO2016/171224 was transformed with the LFABP secretory expression plasmids pPK6lepB(K)_T_LFABP and pPK6lepB(A)_T_LFABP constructed in Example 6(3), to thereby obtain strains YDK010::phoS(W302C)/pPK6lepB(K)_T_LFABP and YDK010::phoS(W302C)/pPK6lepB(A)_T_LFABP. The obtained transformants were each cultured on MMTG liquid medium containing 25 mg/L of kanamycin at 30° C. for 72 hr. After completion of the culture, 5.0 μL of the culture supernatant obtained by centrifuging the culture broth was subjected to reduced SDS-PAGE using NuPAGE(R) 4-12% Bis-Tirs Gel (Thermo Fisher Scientific), and then staining was carried out with SYPRO(R) Ruby (Life Technologies). As a result, two major bands were detected in approximately equal amounts in the strain introduced with pPK6_T_LFABP, in which lepB gene was not amplified, whereas the low molecular weight-side band was significantly increased in both the strains introduced with pPK6lepB(K)_T_LFABP and pPK6lepB(A)_T_LFABP, in which lepB gene was amplified (FIG. 6, panel A, lanes 6-11). As a result of analysis of the N-terminal amino acid sequence, the N-terminal amino acid sequence of LFABP (MSFSGKYQLQ; SEQ ID NO: 53) was observed for the low molecular weight-side band. That is, it was revealed that although a mis-cleaved form of C-terminal side 15 residues of the TorA signal peptide remains in secretory production of LFABP using the TorA signal peptide under non-lepB gene-amplified condition, amplification of lepB gene can promote processing of the TorA signal peptide, to thereby significantly improve production efficiency of LFABP having the correct N-terminal amino acid sequence.

From these results, it was revealed that a mis-cleaved form of C-terminal side 15 residues of the TorA signal peptide remains in secretory production of a heterologous protein using the TorA signal peptide in C. glutamicum, and amplification of lepB gene can promote processing of the TorA signal peptide, to thereby significantly improve production efficiency of the heterologous protein having the correct N-terminal amino acid sequence, regardless of the kind of heterologous protein.

### Example 7

### Evaluation of Effect of lepB Gene-Amplification on Secretory Expression of Transglutaminase Comprising a Pro-Structure Moiety (PTG) Using S1pA Signal Sequence

(1) Construction of Secretory Expression Vector of PTG Using pPK5 Vector

PCR was carried out by using the PTG secretory expression vector pPKSPTG1 disclosed in WO2002/081694 as the template, in combination with primers of SEQ ID NOS: 23 and 24, to amplify a DNA fragment of about 1.8 kbp containing an expression cassette of cspB promoter-S1pA signal sequence-PTG. The primers of SEQ ID NOS: 23 and 24 have been designed to contain a recognition sequence of restriction enzyme KpnI. PCR was carried out with PrimeSTAR(R) HS DNA Polymerase (Takara Bio), and the reaction conditions were according to the protocol recommended by the manufacturer. This DNA fragment was inserted at KpnI site of pPK5 vector disclosed in WO2016/171224 by infusion reaction, to obtain pPK5_S_PTG, which is a secretory expression vector of PTG gene using the SlpA signal sequence (FIG. 1, panel A). The infusion reaction was carried out with In-Fusion(R) HD Cloning Kit (Takara Bio), and the reaction conditions were according to the protocol recommended by the manufacturer. As a result of nucleotide sequencing of the inserted fragment, it was confirmed that the gene as expected was inserted into the vector. Nucleotide sequencing was carried out with BigDye(R) Terminator v3.1 Cycle Sequencing Kit (Applied Biosystems) and 3130 Genetic Analyzer (Applied Biosystems).

(2) Construction of Secretory Expression Vector of PTG Using pPK5lepB Vector

Similar to Example 7(1), PCR was carried out by using the PTG secretory expression vector pPKSPTG1 disclosed in WO2002/081694 as the template, in combination with primers of SEQ ID NOS: 23 and 25 or in combination with primers of SEQ ID NOS: 26 and 27, to amplify a DNA fragment of about 1.8 kbp containing an expression cassette of cspB promoter-S1pA signal sequence-PTG. The primers of SEQ ID NOS: 23 and 25 have been designed to contain a recognition sequence of restriction enzyme KpnI, and the primers of SEQ ID NOS: 26 and 27 have been designed to contain recognition sequences of restriction enzymes ApaI. PCR was carried out with PrimeSTAR(R) HS DNA Polymerase (Takara Bio), and the reaction conditions were according to the protocol recommended by the manufacturer. These DNA fragments were inserted at KpnI or ApaI site of pPK5lepB vector constructed in Example 1 by infusion reaction, to obtain pPK5lepB(K)_S_PTG and pPK5lepB(A)_S_PTG, which are co-expression vectors of lepB gene and PTG secretion using the S1pA signal sequence (FIG. 1, panel B). The letters (K) and (A) in the plasmid names represent the restriction enzyme sites of pPK5lepB vector inserted with the expression cassette of PTG (KpnI site and ApaI site), respectively. The infusion reaction was carried out with In-Fusion(R) HD Cloning Kit (Takara Bio), and the reaction conditions were according to the protocol recommended by the manufacturer. As a result of nucleotide sequencing of the inserted fragments, it was confirmed that the gene as expected was inserted into each vector. Nucleotide sequencing was carried out with BigDye(R) Terminator v3.1 Cycle Sequencing Kit (Applied Biosystems) and 3130 Genetic Analyzer (Applied Biosystems).

(3) Secretory Expression of S1pA Signal Peptide-PTG Under lepB Gene-Amplified Condition and Non lepB Gene-Amplified Condition

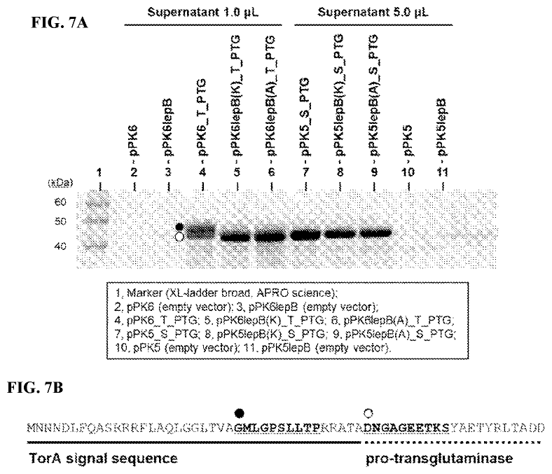

The YDK010::phoS(W302C) strain disclosed in WO2016/171224 was transformed with the PTG secretory expression plasmids using the SlpA signal sequence constructed in Example 7(1) and (2), pPK5_S_PTG, pPK5lepB(K)_S_PTG, and pPK5lepB(A)_S_PTG, to thereby obtain strains YDK010::phoS(W302C)/pPK5_S_PTG, YDK010::phoS(W302C)/pPK5lepB(K)_SP_PTG, and YDK010::phoS(W302C)/pPK5lepB(A)_S_PTG. The obtained transformants were each cultured on MMTG liquid medium containing 25 mg/L of kanamycin at 30° C. for 72 hr. After completion of the culture, 5.0 μL of the culture supernatant obtained by centrifuging the culture broth was subjected to reduced SDS-PAGE using MULTIGEL(R) II mini 15/20 (Cosmo Bio), and then staining was carried out with SYPRO(R) Orange (Thermo Fisher Scientific). As a result, in the case of using the SlpA signal sequence, in contrast to the case of using the TorA signal sequence, a single band of PTG was detected in the strain introduced with pPK5_S_PTG, in which lepB gene was not amplified, and that is, a mis-cleaved form in which a C-terminal partial sequence of the SlpA signal peptide was not detected at all (FIG. 7, panel A, lane 7). Furthermore, the band pattern was not changed in the strains introduced with pPK5lepB(K)_S_PTG and pPK5lepB(A)_S_PTG, in which lepB gene was amplified, and rather the secretory expression amount of PTG was decreased in those strains (FIG. 7, panel A, lanes 8-9).

That is, it was revealed that amplification of lepB gene is not necessarily effective for all signal sequences in secretory production of a heterologous protein using C. glutamicum.

### INDUSTRIAL APPLICABILITY

According to the present invention, heterologous proteins can be efficiently produced by secretory production.

Explanation of Sequence Listing>

SEQ ID NOS:

1: Nucleotide sequence of lepB gene of C. glutamicum ATCC 13869

2: Amino acid sequence of LepB protein of C. glutamicum ATCC 13869

3 to 10: Primers

11: Amino acid sequence of beta-lactamase TEM-116

12: Nucleotide sequence encoding beta-lactamase TEM-116

13 to 14: Primers

15: Amino acid sequence of modified TrxA protein of E. coli

16: Nucleotide sequence encoding modified TrxA protein of E. coli

17 to 18: Primers

19: Amino acid sequence of LFABP of human

20: Nucleotide sequence encoding LFABP of human

21 to 27: Primers

28: Nucleotide sequence of mutant phoS gene of C. glutamicum YDK010

29: Amino acid sequence of mutant PhoS protein of C. glutamicum YDK010

30: Amino acid sequence of PhoS protein of C. glutamicum ATCC 13032

31: Amino acid sequence of PhoS protein of C. glutamicum ATCC 14067

32: Amino acid sequence of PhoS protein of C. callunae

33: Amino acid sequence of PhoS protein of C. crenatum

34: Amino acid sequence of PhoS protein of C. efficiens

35: Nucleotide sequence of phoR gene of C. glutamicum ATCC 13032

36: Amino acid sequence of PhoR protein of C. glutamicum ATCC 13032

37: Nucleotide sequence of cspB gene of C. glutamicum ATCC 13869

38: Amino acid sequence of CspB protein of C. glutamicum ATCC 13869

39: Nucleotide sequence of tatA gene of C. glutamicum ATCC 13032

40: Amino acid sequence of TatA protein of C. glutamicum ATCC 13032

41: Nucleotide sequence of tatB gene of C. glutamicum ATCC 13032

42: Amino acid sequence of TatB protein of C. glutamicum ATCC 13032

43: Nucleotide sequence of tatC gene of C. glutamicum ATCC 13032

44: Amino acid sequence of TatC protein of C. glutamicum ATCC 13032

45: Amino acid sequence of twin-arginine motif

46: Amino acid sequence of TorA signal peptide

47 to 53: N-terminal amino acid sequences

54 to 59: N-terminal amino acid sequences of proteins having signal peptides

1. A method for producing a heterologous protein comprising:
culturing a coryneform bacterium having a genetic construct for secretory expression of the heterologous protein; and
collecting the heterologous protein produced by secretory production,
wherein the coryneform bacterium has been modified so that the activity of a LepB protein is increased,
wherein the genetic construct comprises, in the direction from 5′ to 3′, a promoter sequence that functions in the coryneform bacterium, a nucleic acid sequence encoding a TorA signal peptide, and a nucleic acid sequence encoding the heterologous protein, and
wherein the heterologous protein is expressed as a fusion protein with the TorA signal peptide.

2. The method according to claim 1, wherein the LepB protein is selected from the group consisting of:
(a) a protein comprising the amino acid sequence of SEQ ID NO: 2;
(b) a protein comprising the amino acid sequence of SEQ ID NO: 2, but which includes substitution, deletion, insertion, and/or addition of 1 to 10 amino acid residues, wherein said protein has a signal peptidase activity for the TorA signal peptide; and
(c) a protein comprising an amino acid sequence having an identity of 90% or higher to the amino acid sequence of SEQ ID NO: 2, wherein said protein has a signal peptidase activity for the TorA signal peptide.

3. The method according to claim 1, wherein the activity of the LepB protein is increased by increasing the expression of a lepB gene.

4. The method according to claim 3, wherein the expression of the lepB gene is increased by increasing the copy number of lepB gene and/or modifying an expression control sequence of the lepB gene.

5. The method according to claim 1, wherein the TorA signal peptide is selected from the group consisting of:
(a) a peptide comprising the amino acid sequence of SEQ ID NO: 46;
(b) a peptide comprising the amino acid sequence of SEQ ID NO: 46, but which includes substitution, deletion, insertion, and/or addition of 1 to 3 amino acid residues, wherein said protein has a function as a Tat-dependent signal peptide; and
(c) a peptide comprising an amino acid sequence having an identity of 90% or higher to the amino acid sequence of SEQ ID NO: 46, wherein said protein has a function as a Tat-dependent signal peptide.

6. The method according to claim 1, wherein the TorA signal peptide consists of the amino acid sequence of SEQ ID NO: 46.

7. The method according to claim 1, wherein the heterologous protein produced by secretory production is a heterologous protein from which the TorA signal peptide has been completely removed.

8. The method according to claim 1, wherein the coryneform bacterium has been further modified so as to harbor a phoS gene encoding a mutant PhoS protein.

9. The method according to claim 8, wherein the mutation is replacing an amino acid residue corresponding to the tryptophan residue at position 302 in SEQ ID NO: 29 with an amino acid residue other than an aromatic amino acid and a histidine residue.

10. The method according to claim 9, wherein the amino acid residue other than aromatic an amino acid and a histidine residue is selected from the group consisting of a lysine residue, alanine residue, valine residue, serine residue, cysteine residue, methionine residue, aspartic acid residue, and asparagine residue.

11. The method according to claim 9, wherein the wild-type PhoS protein is selected from the group consisting of:
(a) a protein comprising any of the amino acid sequences of SEQ ID NOS: 29 to 34;
(b) a protein comprising any of the amino acid sequences of SEQ ID NOS: 29 to 34, but which includes substitution, deletion, insertion, and/or addition of 1 to 10 amino acid residues, wherein said protein has a function as a sensor kinase of a PhoRS system; and
(c) a protein comprising an amino acid sequence having an identity of 90% or higher to any of the amino acid sequences of SEQ ID NOS: 29 to 34, wherein said protein has a function as a sensor kinase of a PhoRS system.

12. The method according to claim 1, wherein the coryneform bacterium has been further modified so that the expression of one or more of the genes encoding a Tat secretion system is increased as compared with a non-modified strain.

13. The method according to claim 12, wherein the genes encoding a Tat secretion system consist of a tatA gene, tatB gene, tatC gene, and tatE gene.

14. The method according to claim 1, wherein the coryneform bacterium belongs to the genus Corynebacterium.

15. The method according to claim 14, wherein the coryneform bacterium is Corynebacterium glutamicum.

16. The method according to claim 15, wherein the Corynebacterium glutamicum is derived from Corynebacterium glutamicum AJ12036 (FERM BP-734) or Corynebacterium glutamicum ATCC 13869.

17. The method according to claim 1, wherein the number of molecules of a cell surface layer protein per coryneform bacterium is reduced as compared with a non-modified coryneform bacterium.
